(19) United States
(12) Patent Application Publication
(10) Pub. No.: US 2026/0327264 A1
(43) Pub. Date: Oct. 22, 2026

(54) PEPTIDE INHIBITORS OF INTERLEUKIN-23 RECEPTOR

(71) Applicant: Zealand Pharma A/S, Søborg (DK)

(72) Inventors: Henrik Fischer Munch, Søborg (DK); Octav Caldararu, Søborg (DK); Rasmus Leth, Søborg (DK); Jesper Mosolff Mathiesen, Søborg (DK); Paola Magotti, Søborg (DK)

(21) Appl. No.: 19/484,535

(22) PCT Filed: May 31, 2024
(86) PCT No.: PCT/EP2024/065006

§ 371 Date: Nov. 14, 2025

(30) Foreign Application Priority Data
Jun. 1, 2023 (EP) 23176863.1

(51) Int. Cl. C07K 7/08; A61K 38/00

(52) U.S. Cl. CPC C07K 7/08; A61K 38/00

(57) ABSTRACT

The present invention relates to compounds that are peptide inhibitors of interleukin-23 receptor (IL-23R) and to their use in the treatment or prevention of a variety of diseases, conditions or disorders, including inflammatory diseases, such as inflammatory bowel disease such as Crohn's disease or ulcerative colitis, psoriasis and psoriatic arthritis. The compounds have good physical and chemical stability in the gastrointestinal tract.

### FIELD OF THE INVENTION

The present invention relates to peptide inhibitors of interleukin-23 receptor (IL-23R), and to their medical use in the treatment and/or prevention of a variety of diseases, conditions or disorders, including inflammatory bowel disease (IBD), such as Crohn's disease or ulcerative colitis, psoriasis, psoriatic arthritis, and other conditions and disorders described herein.

### BACKGROUND OF THE INVENTION

Interleukin-23 (IL-23) is a heterodimeric cytokine composed of a unique p19 subunit and the p40 subunit of interleukin-12 (IL-12). IL-12 is a cytokine involved in the development of interferon-gamma (IFN-γ)-producing T helper 1 (Th1) cells. Although both IL-23 and IL-12 contain the p40 subunit, they have different phenotypic properties. Animals deficient in IL-12 are susceptible to inflammatory autoimmune diseases, whereas IL-23 deficient animals are resistant. This is thought to be due to a reduced number of CD4⁺ T cells producing interleukin-6 (IL-6), interleukin-17 (IL-17), and tumor necrosis factor (TNF) in the central nervous system (CNS) of IL-23-deficient animals. Furthermore, in contrast to IL-12 which acts mainly on naive CD4⁺ T cells, IL-23 preferentially acts on memory CD4⁺ T cells.

The receptor that binds IL-23 is the interleukin-23 receptor (IL-23R). IL-23R is a heterodimeric receptor composed of IL-12Rβ1 and IL-23R subunits. Binding of IL-23 to IL-23R activates the JAK-STAT signalling pathway: activating the Janus kinase (JAK) molecules JAK2 and tyrosine kinase 2 (TYK2), as well as the signal transducer and activator of transcription proteins (STATs) STAT1, STAT3, STAT4, and STAT5. STAT4 activation is substantially weaker and different DNA-binding STAT complexes form in response to IL-23 as compared with IL-12. IL-23R associates constitutively with JAK2 and in a ligand-dependent manner with STAT3.

IL-23R is expressed on various adaptive and innate immune cells, including: T-helper 17 (Th17) cells, gamma-delta (γδ) T cells, natural killer (NK) cells, dendritic cells, macrophages, and innate lymphoid cells. These cells are abundantly found in the intestine. In particular, the gene expression and protein levels of IL-23R at the intestine mucosal surface are found to be elevated in inflammatory bowel disease (IBD) patients. It is thought that IL-23 mediates this effect by promoting the development of a pathogenic CD4⁺ T cell population that produces IL6, IL-17, and TNF.

IL-23 production is enriched in the intestine, where it is believed to play a key role in regulating the balance between tolerance and immunity through both T-cell-dependent and independent pathways of intestinal inflammation through effects on Th1 and Th17-associated cytokines. IL-23 is also thought to restrain regulatory T-cell responses in the gut, favoring inflammation. Furthermore, IL-23R polymorphisms have been associated with susceptibility to inflammatory bowel diseases (IBDs), further establishing the critical role of the IL-23 pathway in intestinal homeostasis.

Therefore, IL-23 is thought to play a crucial role in the pathogenesis of autoimmune inflammation and related diseases and disorders, such as multiple sclerosis, asthma, rheumatoid arthritis, psoriasis, psoriatic arthritis, and inflammatory bowel diseases (IBDs), e.g., ulcerative colitis and Crohn's disease. Studies in acute and chronic mouse models of IBD have revealed a primary role of IL-23R and downstream effector cytokines in disease pathogenesis.

The anti-IL-23 antibody Risankizumab (ABBV-006) has been approved for the treatment of inflammatory diseases, including psoriasis, psoriatic arthritis and Crohn's disease, and is also being investigated for the treatment of ulcerative colitis. Protagonist Therapeutics, Inc. had the IL-23 antagonist peptide PTG-200 in Phase II clinical trials for Crohn's disease. Protagonist in collaboration with Janssen Biotech, Inc. also have two second generation IL-23 antagonist peptides in clinical trials: JNJ-77242113 (or JNJ-2113; formerly PN-235) for psoriasis; and PN-232. Protagonist have filed several patent applications in the area of IL-23R inhibitors: WO 2016/011208, WO 2017/011820, WO 2018/022937, WO 2018/136646, WO 2020/014646, WO 2021/007433, WO 2021/146441, WO 2021/146458, WO 2023/288017, WO 2023/288019, WO 2023/288028 and WO 2024/015958. Protagonist also disclose another peptide, Compound C, as an IL-23R inhibitor in WO 2016/011208, WO 2017/011820, and Sayago et al., 2018.

Notably, only single bridging moieties are present in the peptides disclosed by the Protagonist patent applications WO 2016/011208, WO 2017/011820, WO 2018/022937, WO 2018/136646, WO 2020/014646, WO 2021/007433, WO 2021/146441, WO 2021/146458, WO 2023/288028 and WO 2024/015958. None of these applications disclose the use of two bridging moieties to stabilise the peptide inhibitor of IL-23R.

Kong et al., 2020 discloses the development of proteolytically resistant therapeutic peptides for oral administration. The authors generated peptides as inhibitors of coagulation Factor XIa and other peptides as gastrointestinal-protease resistant peptide antagonists of IL-23R. The peptides generated as antagonists of IL-23R comprised of two dithioether bridges (specifically 1,3-dithio-propan-2-one bridges) between two pairs of cysteine residues in the peptide chain. The authors identified peptide 15 as the most promising candidate for further development as an oral treatment of inflammatory disorders such as Crohn's disease on the basis of IL-23R inhibition.

WO 2023/288017 discloses bicyclic (and some tricyclic) peptide inhibitors of IL-23R. The peptides are up to 15 amino acid residues long, all of which have (at least) two bonds bridging between certain amino acid residues. WO 2023/288019 discloses lipidated peptide inhibitors of IL-23R. The peptides are up to 15 amino acid residues long, and either have one or two bonds bridging between certain amino acid residues. Of the peptides with two bridges, one is either a disulfide or dithioether bond and the other is an amide bond. Both applications propose the use of the inhibitors for the treatment of autoimmune inflammation and related diseases and disorders, including IBD, Crohn's disease, ulcerative colitis, psoriasis, and psoriatic arthritis.

However, none of the bridges in the peptides disclosed in Kong et al., 2020, WO 2023/288017 and WO 2023/288019—either by position, type of bond, and/or number of amino acid residues between the bridging amino acids—are the same as the present invention.

Challenges still remain with respect to identifying stable and selective agents that preferentially target the IL-23 pathway, which can be used for the treatment of intestinal inflammation. In particular, the inventors have identified the gastrointestinal stability and IL-23R potency of the peptides disclosed in Kong et al., 2020 could still be further improved for an orally dosed IL-23R peptide blocker. In particular, the most-promising candidate, peptide 15 from Kong et al., 2020, exhibited a lower stability under a simulated intestinal fluid (SIF) assay and a lower potency for IL-23R in comparison with Protagonist's Compound C (Sayago et al., 2018).

Therefore, there remains a need for new therapeutics targeting the IL-23 pathway, which may be used to treat and prevent IL-23-associated diseases, including those associated with autoimmune inflammation in the intestinal tract. Furthermore, compounds and methods for specific targeting of IL-23R from the luminal side of the gut may provide therapeutic benefit to IBD patients suffering from local inflammation of the intestinal tissue.

The inventors have also filed WO 2023/099669 directed to peptide inhibitors of IL-23R. The present invention is directed to further peptide inhibitors with improved stability in the gastrointestinal tract and/or more potent inhibition of IL-23R than the peptides disclosed in WO 2023/099669.

### SUMMARY OF THE INVENTION

The present invention relates to compounds which are peptide inhibitors of interleukin-23 receptor (IL-23R). These compounds exhibit a good combination of properties such as highly potent inhibition of IL-23R and/or high stability in the gastrointestinal tract. Furthermore, the compounds described herein may be useful in the treatment of various diseases, conditions and disorders related to IL-23R such as inflammatory bowel disease, including Crohn's disease and ulcerative colitis, psoriasis and psoriatic arthritis. The compounds described herein exhibit improved properties as compared with the compounds disclosed in WO 2023/099669.

The present invention addresses these needs by providing novel peptide inhibitors that bind IL-23R to inhibit IL-23 mediated signalling. The novel peptide inhibitors are also suitable for oral administration due to their stability in the gastrointestinal tract.

In a first aspect, the invention provides a compound of the formula:

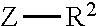

wherein
R² is NHR³ or C(═O)R³, wherein R³ is hydrogen or C₁₋₄ alkyl optionally substituted with a pyridyl ring; and
Z is an amino acid sequence of formula I:

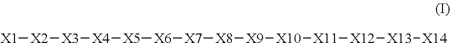

wherein
X1 is absent or is selected from 3-(3-Pyridyl)-Ala or 3-aminopropanoyl;
X2 is selected from the group consisting of 4-aminomethyl-phenylacetyl, an alanine residue substituted by a carbocyclic group or an aromatic or heteroaromatic group selected from the group consisting of phenyl, pyridyl, naphthyl and quinolinyl, each optionally substituted, 3-aminopropanoyl, Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X3 is selected from any amino acid or w-hydroxy-C₂₋₆ alkanoic acid;
X4 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Cys, D-Cys, beta-Cys, D-beta-Cys, homo-Cys, D-homo-Cys, beta-homo-Cys, N-Me-Cys and N-Me-homo-Cys;
X5 is selected from the group consisting of an optionally substituted tryptophan residue, an optionally substituted azatryptophan residue, and an optionally substituted beta-homotryptophan residue;
X6 is selected from the group consisting of an optionally substituted Gln residue, an optionally substituted Lys residue, an optionally substituted Arg residue, an optionally substituted Dab residue, an optionally substituted Orn residue, an optionally substituted Phe residue, Ala, D-Ala, beta-Ala, D-beta-Ala, homo-Ala, D-homo-Ala, beta-homo-Ala, N-Me-Ala, N-Me-homo-Ala, Cit, D-Cit, beta-Cit, D-beta-Cit, homo-Cit, D-homo-Cit, beta-homo-Cit, N-Me-Cit, N-Me-homo-Cit, Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Tyr, D-Tyr, beta-Tyr, D-beta-Tyr, homo-Tyr, D-homo-Tyr, beta-homo-Tyr, N-Me-Tyr, N-Me-homo-Tyr, Val, D-Val, beta-Val, D-beta-Val, homo-Val, D-homo-Val, beta-homo-Val, N-Me-Val, N-Me-homo-Val or His, D-His, beta-His, D-beta-His, homo-His, D-homo-His, beta-homo-His, N-Me-His and N-Me-homo-His;
X7 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Cys, D-Cys, beta-Cys, D-beta-Cys, homo-Cys, D-homo-Cys, beta-homo-Cys, N-Me-Cys and N-Me-homo-Cys;
X8 is selected from the group consisting of an optionally substituted tryptophan residue, an optionally substituted azatryptophan residue, an optionally substituted beta-homotryptophan residue, an optionally substituted tyrosine residue, an optionally substituted phenylalanine residue, an optionally substituted homophenylalanine residue, and an alanine residue substituted by an optionally substituted carbocylic group or an aromatic group selected from the group consisting of phenyl, pyridyl, naphthyl and quinolinyl, each optionally substituted;
X9 is selected from the group consisting of an optionally substituted tryptophan residue, an optionally substituted azatryptophan residue, an optionally substituted beta-homotryptophan residue, an optionally substituted alanine residue, an optionally substituted phenylalanine and an optionally substituted tyrosine residue;
X10 is selected from the group consisting of Val, D-Val, beta-Val, D-beta-Val, homo-Val, D-homo-Val, beta-homo-Val, N-Me-Val, N-Me-homo-Val, 2-Me-Val,
Gly, beta-Gly, homo-Gly, beta-homo-Gly, N-Me-Gly, N-Me-homo-Gly,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, 2-Me-Lys,
Aib, D-Aib, beta-Aib, D-beta-Aib, homo-Aib, D-homo-Aib, beta-homo-Aib, N-Me-Aib, N-Me-homo-Aib,
Ala, D-Ala, beta-Ala, D-beta-Ala, homo-Ala, D-homo-Ala, beta-homo-Ala, N-Me-Ala, N-Me-homo-Ala,
Leu, D-Leu, beta-Leu, D-beta-Leu, homo-Leu, D-homo-Leu, beta-homo-Leu, N-Me-Leu, N-Me-homo-Leu, 2-Me-Leu,
Ile, D-Ile, beta-Ile, D-beta-Ile, homo-Ile, D-homo-Ile, beta-homo-Ile, N-Me-Ile, N-Me-homo-Ile,
and a carbocyclic or heterocyclic ring having an amino substituent and a carbonyl substituent;
X11 is selected from the group consisting of Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, Lys(Me),
Lys(Gly), LAsp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X12 is selected from the group consisting of an optionally substituted Phe residue, an optionally substituted Tyr residue, an optionally substituted His residue, an alanine residue substituted by a carbocyclic group or an aromatic or heteroaromatic group selected from the group consisting of phenyl, pyridyl, naphthyl and quinolinyl, each optionally substituted, Dab, D-Dab, iso-Dab, D-iso-Dab, beta-Dab, D-beta-Dab, homo-Dab, D-homo-Dab, beta-homo-Dab, N-Me-Dab, N-Me-homo-Dab, Gly, beta-Gly, homo-Gly, beta-homo-Gly, N-Me-Gly, N-Me-homo-Gly, Pro, 5-aminopentanoyl, 4-aminopiperidin-4-carbonyl, (R,S)-Imidazolidin-2-carbonyl, 3-aminopropanoyl, Gly-CF3, D-Gly-CF3, Nle, Gln, D-Gln, iso-Gin, D-iso-Gln, beta-Gin, D-beta-Gln, homo-Gin, D-homo-Gln, beta-homo-Gin, N-Me-Gin, N-Me-homo-Gln, THP, Ser, D-Ser, beta-Ser, D-beta-Ser, homo-Ser, D-homo-Ser, beta-homo-Ser, N-Me-Ser, 2-Me-Ser, N-Me-homo-Ser, Ser(OMe), 3-aminotetrahydrofuran-3-carbonyl, THP, Arg, D-Arg, beta-Arg, D-beta-Arg, homo-Arg, D-homo-Arg, beta-homo-Arg, N-Me-Arg, N-Me-homo-Arg, Thr, D-Thr, beta-Thr, D-beta-Thr, homo-Thr, D-homo-Thr, beta-homo-Thr, N-Me-Thr, N-Me-homo-Thr Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Asn, D-Asn, beta-Asn, D-beta-Asn, homo-Asn, D-homo-Asn, beta-homo-Asn, N-Me-Asn, N-Me-homo-Asn, GABA, 2-(trimethyl-2-aminoethoxy)ethoxy]propyl and Lys wherein the side chain —NH₂ of the Lys is substituted with —C(═O)(CH₂)ₙR^{K} wherein n is 0-2 and R^{K} is imidazolyl, pyrimidyl, or pyridyl optionally substituted with F;
X13 is selected from the group consisting of an optionally substituted His residue, an optionally substituted Phe residue, an alanine residue substituted by a carbocyclic group or an aromatic or heteroaromatic group selected from the group consisting of phenyl, pyridyl, naphthyl and quinolinyl, each optionally substituted, Asn, D-Asn, beta-Asn, D-beta-Asn, homo-Asn, D-homo-Asn, beta-homo-Asn, N-Me-Asn, N-Me-homo-Asn, Gly, beta-Gly, homo-Gly, beta-homo-Gly, N-Me-Gly, N-Me-homo-Gly, and Dab, Orn or Lys wherein the side chain —NH₂ is substituted with —C(═O)(CH₂)ₙR^{K} wherein n is 0-2 and R^{K} is imidazolyl, pyrimidyl, or pyridyl optionally substituted with F, or is absent; and
X14 is absent or is selected from the group consisting of Sar, an optionally substituted His residue, Leu, D-Leu, beta-Leu, D-beta-Leu, homo-Leu, D-homo-Leu, beta-homo-Leu, N-Me-Leu, N-Me-homo-Leu, 2-Me-Leu, and an alanine residue substituted by a carbocyclic group or an aromatic or heteroaromatic group selected from the group consisting of phenyl, pyridyl, naphthyl and quinolinyl, each optionally substituted;
wherein
(i) the amino acid residue at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof;
wherein the compound is not:

| | |
|---|---|
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| Ref 112 | [Dab](1c)*SC(2a)WQC(2a)[Y(2-aminoethoxy)][2-Nal][2-Me- |
| | Leu]E(1c)[{d}R]-[NH2] |
| Ref 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| Ref 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| Ref 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| Ref 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| Ref 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| Ref 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| Ref 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| Ref 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| Ref 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| Ref 130 | [4-Aminomethyl-phenylacetyl](1c)*SE(2c)WQ[Dab](2c)F[2-Nal][2-Me- |
| | Leu]E(1c)[Dab]-[NH2] |
| Ref 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| Ref 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| Ref 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| Ref 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| Ref 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| Ref 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| Ref 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab]-[NH2] |
| Ref 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab]-[NH2] |
| Ref 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab]-[NH2] |
| Ref 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| Ref 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| Ref 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| Ref 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| Ref 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| Ref 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| Ref 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| Ref 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| Ref 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| Ref 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| Ref 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| Ref 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| Ref 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| Ref 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| Ref 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala]-[NH2] |
| Ref 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| Ref 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| Ref 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| Ref 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| Ref 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

Above reference compounds were disclosed in WO2023/099669.

In a second aspect, the invention provides a compound of the formula:

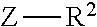

wherein
R² is NHR³ wherein R³ is hydrogen or C₁₋₄ alkyl optionally substituted with a pyridyl ring; and
Z is an amino acid sequence of formula I:

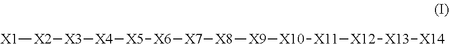

wherein
X1 is absent or is selected from 3-(3-Pyridyl)-Ala or 3-aminopropanoyl;
X2 is selected from the group consisting of 4-aminomethyl-phenylacetyl, 3-(3-Pyridyl)-Ala, and 3-aminopropanoyl;
X3 is selected from the group consisting of Ser, Ala, Phe, and N-Me-Ser;
X4 is Glu or Cys;
X5 is Trp;
X6 is selected from the group consisting of Gln, Q(Me), Q(2Me), Q(pyrrolidin), Ala, Phe, Lys(Ac), Dab(Ac), Cit, and Orn;
X7 is Dab or Cys;
X8 is selected from the group consisting of Y(2-aminoethoxy), homo-Phe, 7-AzaTrp, beta-homo-Trp, and 7-F-Trp;
X9 is selected from the group consisting of 2-Nal, 7-AzaTrp, beta-homo-Trp, and Cyclopropyl-Ala;
X10 is 2-Me-Leu or Aib;
X11 is Glu;
X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser;
X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent; and
X14 is absent or is selected from the group consisting of Sar, D-His, beta-homo-Leu, and 3-(3-Pyridyl)-Ala;
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the compound is not:

| | |
|---|---|
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

Reference compounds Ref 80, Ref 81, Ref 82, Ref 120, Ref 122, Ref 125, Ref 141, and Ref 146 were disclosed in WO2023/099669.

The present invention is not directed to the compounds disclosed in WO2023/099669.

In some embodiments, the compound is not when X1 is absent; X2 is 4-aminomethyl-phenylacetyl; X3 is Ser or N-Me-Ser; X6 is Gln or Q(pyrrolidin); X8 is Y(2-aminoethoxy); X9 is 2-Nal, X10 is 2-Me-Leu; X12 is Dab, D-Arg, or Ser; X13 is 3-(3-Pyridyl)-Ala or absent; X14 is absent; and R² is NH₂.

In some embodiments, X1 is absent.

In some embodiments, X2 is 4-aminomethyl-phenylacetyl.

In some embodiments, X3 is Ser or Ala. In some embodiments, X3 is Ser.

In some embodiments, X4 is Glu, X7 is Dab, and wherein X4 and X7 together form a lactam bridge.

In some embodiments, X6 is Gln.

In some embodiments, X8 is Y(2-aminoethoxy).

In some embodiments, X9 is 2-Nal.

In some embodiments, X10 is 2-Me-Leu.

In some embodiments, X12 is Dab.

In some embodiments, X13 is 3-(3-Pyridyl)-Ala or is absent. In some embodiments, X13 is 3-(3-Pyridyl)-Ala.

In some embodiments, X14 is absent.

In some embodiments, R² is NHR³ wherein R³ is hydrogen or C₁₋₄ alkyl. In some embodiments, R² is NH₂. In some other embodiments, R² is NHMe.

In some embodiments, X4 and X7 are both Cys, and X4 and X7 together form a dithioether bridge, wherein the dithioether bridge between X4 and X7 is of the formula —S-L-Y-L-S—, wherein:
each S is a sulfur atom and is part of the amino acid residue at X4 and X7;
each L is independently C₁₋₄ alkylene; and
Y is either absent or C(═O).

In some embodiments, L is independently C₁₋₂ alkylene. In some embodiments, L is methylene. In some embodiments, Y is C(═O).

In some embodiments, the dithioether bridge between X4 and X7 is of the formula —SCH₂C(═O)CH₂S—, wherein each S is a sulfur atom and is part of the amino acid residue at X4 and X7.

In some embodiments, X1 is absent and X2 is 4-aminomethyl-phenylacetyl.

In some embodiments, X1 is absent; X2 is 4-aminomethyl-phenylacetyl; X8 is Y(2-aminoethoxy); X9 is 2-Nal; and X10 is 2-Me-Leu.

In some embodiments, X1 is absent; X2 is 4-aminomethyl-phenylacetyl; X3 is Ser or Ala; X8 is Y(2-aminoethoxy); X9 is 2-Nal; and X10 is 2-Me-Leu.

In some embodiments, X1 is absent; X2 is 4-aminomethyl-phenylacetyl; X3 is Ser or Ala; X6 is Gln; X8 is Y(2-aminoethoxy); X9 is 2-Nal; and X10 is 2-Me-Leu.

In some embodiments, X13 is 3-(3-Pyridyl)-Ala and X14 is absent.

In some embodiments, X13 is 3-(3-Pyridyl)-Ala, X14 is absent, and R² is NHMe.

In some embodiments, Z is an amino acid sequence selected from the group consisting of a sequence listed in Table 1-1a.

In some embodiments, the compound is selected from a compound from Table 1-1, or a pharmaceutically acceptable salt or solvate thereof.

The invention further provides a composition comprising a compound as described above. The composition may be a pharmaceutical composition, and may comprise a pharmaceutically acceptable carrier, excipient or vehicle.

The invention further provides a method for the synthesis of a compound as described above. The method may comprise the steps of synthesising the peptide by solid-phase or liquid-phase methodology, and optionally isolating and/or purifying the final product, and optionally further comprising the step of forming an amide bond between the amino acid residues at position X1 or at position X2 when X1 is absent and at position X11, and optionally further comprising the step of forming an amide bond or forming two thioether bonds with a linker between the amino acid residues at positions X4 and X7.

The invention further provides a compound of the invention, or a pharmaceutical compositions comprising said compound, for use in a method of medical treatment.

The invention also provides a compound of the invention, or a pharmaceutical composition comprising said compound, for use in a method of prevention or treatment of inflammatory bowel disease (IBD) such as Crohn's Disease or ulcerative colitis, psoriasis, psoriatic arthritis, and combinations thereof. In some embodiments, the condition is inflammatory bowel disease (IBD) and/or psoriasis.

The invention also provides use of a compound of the invention, or the pharmaceutical composition comprising said compound, in the manufacture of a medicament for the prevention or treatment of inflammatory bowel disease (IBD), such as Crohn's Disease or ulcerative colitis, psoriasis, psoriatic arthritis, and combinations thereof. In some embodiments, the condition is inflammatory bowel disease (IBD) and/or psoriasis.

The invention also provides a method of prevention or treatment of inflammatory bowel disease (IBD) such as Crohn's Disease or ulcerative colitis, psoriasis, psoriatic arthritis, and combinations thereof; which comprises administering to the subject a therapeutically effective amount of the compound of the invention, or the pharmaceutical composition comprising said compound. In some embodiments, the condition is inflammatory bowel disease (IBD) and/or psoriasis.

Further aspects and embodiments of the present invention will become apparent from the disclosure below.

### DETAILED DESCRIPTION OF THE INVENTION

### Definitions

Unless otherwise defined herein, scientific and technical terms used herein shall have the meanings that are commonly understood by those of ordinary skill in the art. Generally, nomenclature employed herein in connection with techniques of chemistry, molecular biology, cell and cancer biology, immunology, microbiology, pharmacology, and protein and nucleic acid chemistry, described herein, is that well known and commonly used in the art.

All publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer or component, or of a stated group of integers or components, but not the exclusion of any other integer or component or group of integers or components.

The singular forms “a”, “an”, and “the” include the plurals unless the context clearly dictates otherwise.

The term “including” is used to mean “including but not limited to”. “Including” and “including but not limited to” are used interchangeably.

The terms “patient”, “subject”, and “individual” may be used interchangeably and may refer to either a human or a non-human animal. Subjects are typically mammals, including humans, non-human primates (including great apes, Old World monkeys and New World monkeys), livestock animals (e.g., bovines, porcines), companion animals (e.g., canines, felines) and rodents (e.g., mice and rats).

As used herein, the term “pharmaceutically acceptable salt” is intended to indicate a salt which is not harmful to a patient or subject to which the salt in question is administered. It may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other examples of pharmaceutically acceptable salts are described in “Remington's Pharmaceutical Sciences”, 17^{th} edition, Alfonso R. Gennaro (Ed.), Mark Publishing Company, Easton, PA, USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66: 2 (1977).

The term “solvate” in the context of the present invention refers to a complex of defined stoichiometry formed between a solute (in casu, a peptide or pharmaceutically acceptable salt thereof according to the invention) and a solvent. The solvent in this connection may, for example, be water, ethanol or another pharmaceutically acceptable—typically small-molecular—organic species, such as, but not limited to, acetic acid or lactic acid. When the solvent in question is water, such a solvate is normally referred to as a hydrate.

The terms “antagonist” and “inhibitor” as employed in the context of the invention refer to a substance that inhibits the receptor type in question, typically by binding to it (i.e. as a ligand) and blocking it.

Each embodiment of the invention described herein may be taken alone or in combination with one or more other embodiments of the invention.

The term “therapeutically effective amount” or “effective amount” as used herein in the context of the above-described methods of treatment or other therapeutic interventions according to the invention refers to an amount that is sufficient to cure, ameliorate, alleviate or partially arrest the clinical manifestations of the particular disease, disorder or condition that is the object of the treatment or other therapeutic intervention in question e.g. as measured by established clinical endpoints or other biomarkers (established or experimental). A therapeutically relevant amount may be determined empirically by one skilled in the art based on the indication being treated or prevented and the subject to whom the therapeutically relevant amount is being administered. For example, the skilled worker may measure one or more of the clinically relevant indicators of bioactivity described herein, e.g. myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), Claudin 8 (CLDN8), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins, as described in WO 2018/089693. The skilled worker may determine a clinically relevant amount through in vitro or in vivo measurements.

An amount adequate to accomplish any or all of these effects is defined as a therapeutically effective amount. The administered amount and the method of administration can be tailored to achieve optimal efficacy. An amount effective for a given purpose will depend, inter alia, on the severity of the disease, disorder or condition that is the object of the particular treatment or other therapeutic intervention, on the body weight and general condition of the subject in question, on diet, on possible concurrent medication, and on other factors well known to those skilled in the medical arts. Determination of an appropriate dosage size and dosing regimen most appropriate for administration of a peptide or pharmaceutically acceptable salt or solvate thereof according to the invention to a human may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are well known to the skilled person.

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g. a human) in need of treatment may thus be a subject already afflicted with the disease or disorder in question. The term “treatment” includes inhibition or reduction of an increase in severity of a pathological state or symptoms (e.g. inflammation) relative to the absence of treatment, and is not necessarily meant to imply complete cessation of the relevant disease, disorder or condition.

The terms “prevention” and grammatical variants thereof (e.g., “prevented”, “preventing”, “prevent”) as employed in the present context refer to an approach for hindering or preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g. a human) in need of “prevention” may thus be a subject not yet afflicted with the disease or disorder in question. The term “prevention” thus includes inhibiting or slowing the onset of disease relative to the absence of treatment, and is not necessarily meant to imply permanent prevention of the relevant disease, disorder or condition.

### Amino Acids Nomenclature

The term “amino acid” is an organic compound that, in isolation, contains an amino or amine group (—NH₂ or —NHR) and a carboxylic acid (—COOH) group. As is known to the person skilled in the art, the amine and carboxylic acid groups of amino acid residues react together to form peptides having an amide bond, also referred to as a peptide linkage, of the formula —NH—C(═O)— or —NR—C(═O)—.

The term “amino acid” is thus not limited to including natural and unnatural alpha and beta amino-acids but also includes (when forming part of a peptide) residues such as 3-aminopropanoyl and 4-aminobutanoyl. The term also includes cyclic structures such as carbocyclic and heterocyclic structures, having an amine and a carboxylic acid functionality. The carbonyl of the carboxylic acid group may be further functionalised, such as converted to —CF₃ in Gly-CF3 and D-Gly-CF3. This further functionalisation may occur before, during, or after peptide coupling with the other amino acid residues in the peptide chain.

Some amino acids described herein have the amine and carboxylic acid groups attached to the same carbon, called alpha (a) amino acids. Some amino acids described herein have the amine and carboxylic acid groups 1, 2, 3, 4, 5, or 6 carbon atoms away. For example, beta-homo-Trp and beta-homo-Leu have the amine and the carboxylic acid groups 1 carbon away from each other, such that the carbon connected to the amine group and the carbon connected to the carboxylic acid group are adjacent to one another.

Some amino acids described herein have a side chain specific to each amino acid. The side chain may also be further functionalised.

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). In the case of certain less common or non-naturally occurring amino acids (i.e. amino acids other than the 20 encoded by the standard mammalian genetic code), unless they are referred to by their full name (e.g. ornithine, etc.), frequently employed three- or four-character codes are employed for residues thereof, including 2-Nal (3-(2-naphthyl)-alanine).

Unless otherwise indicated, reference is made to both the L and D-isomeric forms of the amino acids in question. In one embodiment, unless otherwise stated, the amino acids referred to herein are in their L isomeric form. In one embodiment, unless otherwise stated, the amino acids referred to herein are in their D isomeric form. In a preferred embodiment, unless otherwise stated, the amino acids referred to herein are in their L isomeric form.

Unless otherwise indicated, reference is made to both the homo and non-homo forms of the amino acids in question. As evident from Table A below, the prefix “homo” to the name of an amino acid indicates the addition of a methylene group to the α-carbon of an amino acid. In one embodiment, unless otherwise stated, the amino acids referred to herein are in their non-homo form. In one embodiment, unless otherwise stated, the amino acids referred to herein are in their homo-form. In a preferred embodiment, unless otherwise stated, the amino acids referred to herein are in their non-form.

Unless otherwise indicated, reference is made to both the alpha and beta forms of the amino acids in question. As evident from Table A below, the prefix “beta” to the name of an amino acid indicates that the carbon skeleton has been lengthened by insertion of one carbon atom immediately after the acid group of the amino acid backbone. In one embodiment, unless otherwise stated, the amino acids referred to herein are in their alpha form. In one embodiment, unless otherwise stated, the amino acids referred to herein are in their beta-form. In a preferred embodiment, unless otherwise stated, the amino acids referred to herein are in their alpha form.

Amino acid residues are amino acid moieties within a peptide chain. Unnatural amino acid residues may be identified as the fragment of the unnatural amino acid defined in a peptide chain (for example, the unnatural amino acid 3-aminopropanoic acid may be identified as the unnatural amino acid residue 3-aminopropanoyl in a peptide chain).

Additional exemplary abbreviations for amino acid residues are described in Table A.

TABLE A

| Amino Acid Residue | Structure |
|---|---|
| 3-(3-Pyridyl)-Ala also known as: 3-(3-pyridyl)-L- alanine, 3-Pal, or (2S)-2-amino-3- (pyridin-3-yl)propanoic acid | 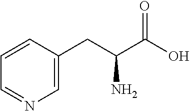 [SMILES: N[C@@H](Cc1cccnc1)C(=O)O] |
| 3-aminopropanoic acid | 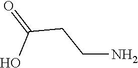 |
| 4-aminomethyl-phenylacetic acid | 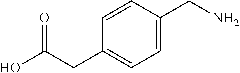 [SMILES: NCc1ccc(CC(=O)O)cc1] |
| N-Me-Ser also known as: N-methyl-L-serine or (2S)-3-hydroxy-2-(methyl- amino)propanoic acid | 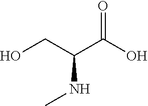 |
| Q(Me) also known as: (2S)-2-amino-5- (methylamino)-5-oxo-pentanoic acid | 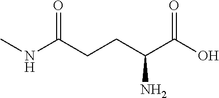 |
| Q(2Me) also known as: (2S)-2-amino-5- (dimethylamino)-5-oxo-pentanoic acid | 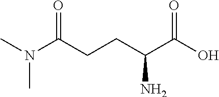 |
| Q(pyrrolidin) also known as: (2S)-2-amino-5- oxo-5-pyrrolidin-1-yl-pentanoic acid | 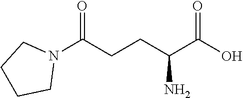 [SMILES: N[C@@H](CCC(=O)N1CCCC1)C(=O)O] |
| Lys(Ac) or K(Ac) also known as: acetyllysine, N(6)-acetyl-L-lysine or (2S)-6-acetamido-2-amino- hexanoic acid | 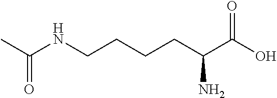 |
| Dab(Ac) also known as: (2S)-4-acetamido- 2-amino-butanoic acid | 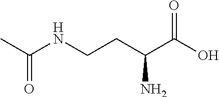 |
| Cit also known as: L-citrulline or (2S)- 2-amino-5-ureido-pentanoic acid | 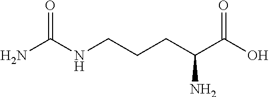 [SMILES: NC(=O)NCCC[C@H](N)C(=O)O] |
| Orn also known as: L-ornithine or (2S)- 2,5-diaminopentanoic acid | 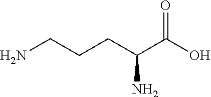 |
| Dab (also iso-Dab - see Table B) also known as: (2S)-2,4-diamino- butanoic acid | 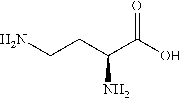 |
| D-Dab Also known as {d}Dab or D-2,4- diaminobutanoic acid or (2R)-2,4- diaminobutanoic acid | 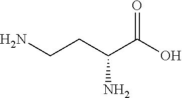 |
| Dpr Also known as: L-diaminopropionic acid | 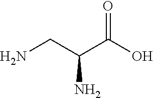 |
| Y(2-aminoethoxy) also known as: (2S)-2-amino-3-[4- (2-aminoethoxy)phenyl]propanoic acid | 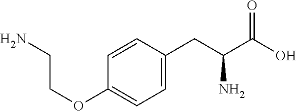 [SMILES: NCCOc1ccc(C[C@H](N)C(=O)O)cc1] |
| homo-Phe or hPhe also known as: L-Homophenyl- alanine or (2S)-2-amino-4-phenyl- butanoic acid | 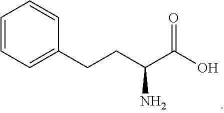 [SMILES: N[C@@H](CCc1ccccc1)C(=O)O] |
| 7-AzaTrp also known as: (2S)-2-amino-3- (1H-pyrrolo[2,3-b]pyridin-3-yl)- propanoic acid | 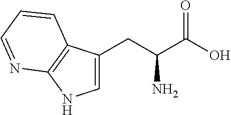 [SMILES: N[C@@H](Cc1c[nH]c2ncccc12)C(=O)O] |
| beta-homo-Trp or beta-h Trp also known as: L-beta-homotrypto- phan, L-β-homotryptophan, or (3S)-3-amino-4-(1H-indol-3-yl)- butanoic acid | 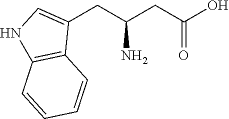 [SMILES: N[C@H](CC(=O)O)Cc1c[nH]c2ccccc12] |
| 7-F-Trp also known as: (2S)-2-amino-3- (7-fluoro-1H-indol-3-yl)propanoic acid | 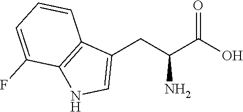 [SMILES: N[C@@H](Cc1c[nH]c2c(F)cccc12)C(=O)O] |
| 7-Me-Trp also known as: (2S)-2-amino-3-(7- methyl-1H-indol-3-yl)propanoic acid | 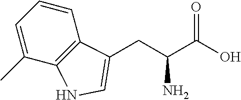 [SMILES: Cc1cccc2c(C[C@H](N)C(=O)O)c[nH]c12] |
| 2-Nal also known as: 3-(2-naphthyl)-L- alanine or (2S)-2-amino-3-(2- naphthyl)propanoic acid | 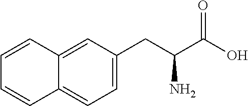 [SMILES: N[C@@H](Cc1ccc2ccccc2c1)C(=O)O] |
| Cyclopropyl-Ala also known as: cyclopropyl-L- alanine or (2S)-2-amino-3-cyclo- propyl-propanoic acid | 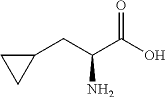 |
| 2-Me-Leu also known as: 2-methyl-L-leucine, alpha-methylleucine, a-methyl- leucine, or (2S)-2-amino-2,4- dimethylpentanoic acid | 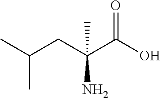 |
| Aib also known as: 2-aminoisobutyric acid, alpha-methylalanine, a-meth- ylalanine, or 2-methylalanine, or 2-amino-2-methyl-propanoic acid | 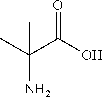 |
| D-Arg or {d}R also known as: D-arginine or (2R)- 2-amino-5-guanidino-pentanoic acid | 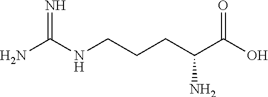 [SMILES: N=C(N)NCCC[C@@H](N)C(=O)O] |
| Gly-CF3 | 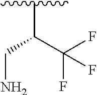 [SMILES: *[C@@H](CN)C(F)(F)F] |
| D-Gly-CF3 | 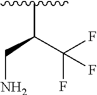 [SMILES: *[C@H](CN)C(F)(F)F] |
| Nle also known as: L-norleucine or (2S)-2-aminohexanoic acid | 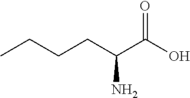 |
| THP also known as: 4-aminotetrahydro- pyran-4-carboxylic acid | 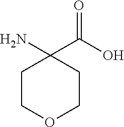 |
| D-Ser or {d}S also known as: D-serine or (2R)-2- amino-3-hydroxy-propanoic acid | 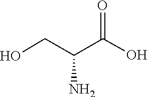 |
| 2-Me-Ser also known as: 2-methyl-L-serine, alpha-methyl-L-serine (2S)-2- amino-3-hydroxy-2-methyl- propanoic acid | 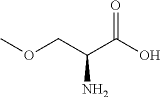 |
| Ser(OMe) also known as: O-methyl-L-serine or (2S)-2-amino-3-methoxy- propanoic acid | 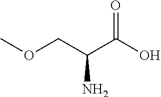 |
| homo-Ser or hSer also known as: L-homoserine or (2S)-2-amino-4-hydroxybutanoic acid | 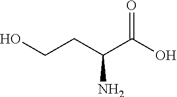 |
| D-3-(3-Pyridyl)-Ala or {d}3-(3-Pyridyl)-Ala also known as: 3-(3-pyridyl)-D- alanine, D-3-Pal, or (2R)-2-amino- 3-(pyridin-3-yl)propanoic acid | 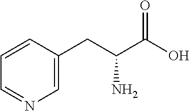 [SMILES: N[C@H](Cc1cccnc1)C(=O)O] |
| 2-Me-3-(3-Pyridyl)-Ala also known as: 2-Me-3-Pal, or (2S)- 2-amino-2-methyl-3-(3- pyridyl)propanoic acid | 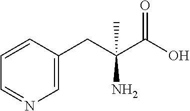 |
| N-Me-3-(3-Pyridyl)-Ala also known as: N-Me-3-Pal, or (2S)-2-(methylamino)-3-(3- pyridyl)propanoic acid | 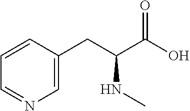 |
| 3-(3,5-Pyrimidyl)-Ala also known as: (2S)-2-amino-3- pyrimidin-5-yl-propanoic acid | 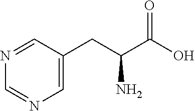 [SMILES: N[C@@H](Cc1cncnc1)C(=O)O] |
| D-His or {d}H also known as: D-histidine or (2R)-2-amino-3-(1H-imidazol- 4-yl)propanoic acid | 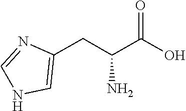 [SMILES: N[C@H](Cc1c[nH]cn1)C(=O)O] |
| His(Me) or H(Me) or His(1-Me) or H(1-Me) also known as: 1-methyl-L-histi- dine or (2S)-2-amino-3-(1-meth- ylimidazol-4-yl)propanoic acid | 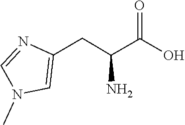 |
| Sar also known as: sarcosine, N- methyl-glycine, or 2-(methyl- amino)-acetic acid | 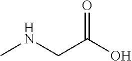 |
| beta-homo-Leu or beta-hLeu also known as: L-beta-homo- leucine, L-B-homoleucine, or (3S)-3-amino-5-methyl-hexanoic acid | 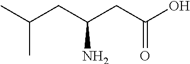 |
| F(4-morpholine) also known as: 4-morpholine-L- phenylalanine or (2S)-2-Amino- 3-(4-morpholin-4-ylphenyl)- propanoic Acid | 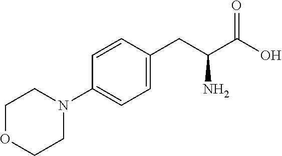 [SMILES: N[C@@H](Cc1ccc(N2CCOCC2)cc1)C(=O)O] |
| N-Me-Ala Also known as: N-methyl-L- alanine | 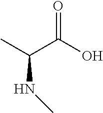 |
| 3-quinolinylalanine | 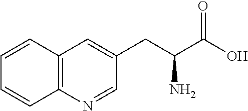 [SMILES: N[C@@H](Cc1cnc2ccccc2c1)C(=O)O] |
| Y(2-aminoethoxy)(N(Me)2) | 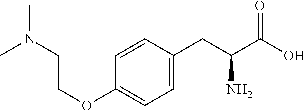 [SMILES: CN(C)CCOc1ccc(C[C@H](N)C(=O)O)cc1] |
| Y(nPentylamine)(N⁺(Me)3) | 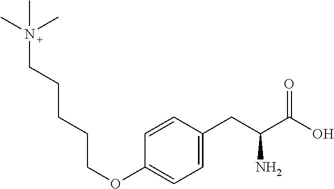 |
| Y(2-trimethyl-PEG2) | 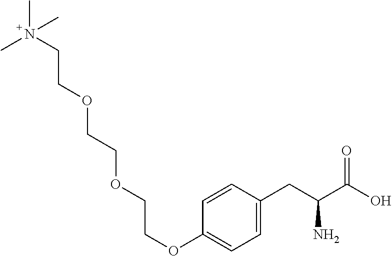 |
| 3-hydroxy propanoic acid | 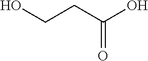 |
| 2-amino-6-carboxyhexanoic acid | 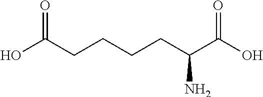 [SMILES: N[C@@H](CCCCC(=O)O)C(=O)O] |
| 3-aminotetrahydrofuran-3-carbox- ylic acid | 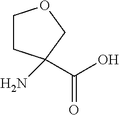 |
| 4-aminopiperidin-4-carboxylic acid | 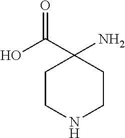 |
| (R,S)-Imidazolidin-2-carboxylic acid | 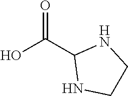 |

Using the above Table A and the below Table A1, the skilled person would be able to derive the structure of any D-, beta-, homo-, homo-beta- and N-Me analogues (and combinations thereof) of the amino acid residues disclosed herein. In particular, Table A1 outlines the relevant analogues for a lysine amino acid, which can subsequently be applied to derive the corresponding structures for any equivalent amino acid analogue disclosed herein.

TABLE A1

| Amino Acid Residue | Structure |
|---|---|
| Lys Also known as L-lysine or 2S)-2,6- Diaminohexanoic acid (L-lysine) | 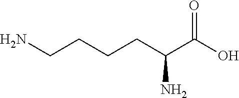 |
| D-Lys or {d}K also known as: D-lysine or (2R)- 2,6-diaminohexanoic acid | 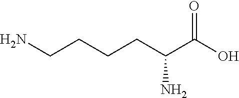 |
| beta-Lys or bLys also known as: L-beta-lysine or (3S)-3,6-diaminohexanoic acid | 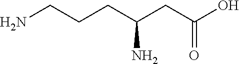 |
| D-beta-Lys or {d}bLys also known as: D-beta-lysine or (3R)-3,6-diaminohexanoic acid | 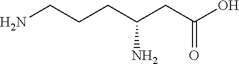 |
| homo-Lys or hLys also known as: L-homolysine or (2S)-2-amino-7-amino-heptanoic acid | 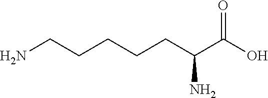 [SMILES: NCCCCC[C@H](N)C(=O)O] |
| D-homo-Lys or {d}hLys also known as: D-homolysine or (2R)-2-amino-7-amino-heptanoic acid | 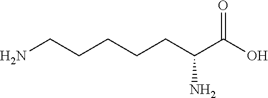 [SMILES: NCCCCC[C@@H](N)C(=O)O] |
| beta-homo-Lys or beta-hLys also known as: L-beta-homo- lysine, L-B-homolysine, or (3S)- 3,7-diaminoheptanoic acid | 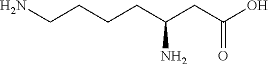 [SMILES: NCCCC[C@H](N)CC(=O)O] |
| N-Me-Lys also known as: N2-methyl-L- lysine or (2S)-6-amino-2-(meth- ylamino)hexanoic acid | 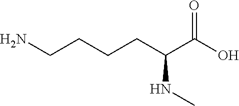 |
| N-Me-homo-Lys also known as: N2-methyl-L- homolysine | 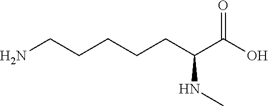 |

Linear peptides are written from N-terminus to C-terminus, left to right.

Unnatural (or non-naturally occurring) amino acids and unnatural (or non-naturally occurring) amino acid residues are amino acids and amino acid residues that do not naturally occur in peptide chains. Unnatural amino acids may be formed as secondary metabolites in bacteria, fungi, plants, or marine organisms, or they can be synthesised chemically.

Unless otherwise stated, the peptide backbone (that is, the amide bonds “-” between X1-X2-X3-X4-X5-X6-X7-X8-X9-X10-X11-X12-X13-X14 in the peptide chain) is formed by the amino acid residues joined by amide bonds via their terminal —NH₂ and —COOH groups. That is, the “terminal —NH₂ group” is the alpha-amine group for alpha amino acids or beta-amine group for beta amino acids; and the “terminal —COOH group” is the alpha-carboxylic acid group for the alpha amino acids. The skilled person thus understands that the amino acids within the peptide chains (i.e., those specified for at least X3, X4, X5, X6, X7, X8, X9, X10, X11 and X12) will be in the form —NH—X—C(═O)—, wherein X represents the amino acid structure between the amine and carboxylic acids residues that form the amide backbone of the peptide chain.

Where the amino acid residue comprises two or more amine groups (—NH₂, such as Lys and Dab) or carboxylic acid groups (—COOH, such as Glu), the peptide backbone may instead be formed using the side chain of the amino acid residue.

For example, the following nomenclature in Table B is used to distinguish the use of the terminal and the side chain —NH₂ and —COOH groups.

TABLE B

| Amino Acid Residue | Residue in peptide chain | Iso-residue in peptide chain |
|---|---|---|
| 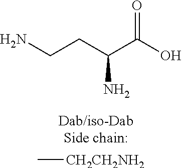 | 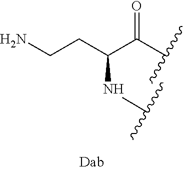 [SMILES: *N[C@@H](CCN)C(*)=O] | 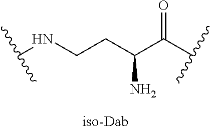 [SMILES: *NCC[C@H](N)C(*)=O] |

It will be understood that the above Table B taken in conjunction with Table A and Table A1 can be used to derive the structures of any corresponding iso analogues disclosed herein.

The C-terminus of the peptide may be derivatised with an alkyl group substituted with a pyridyl group to improve gastrointestinal stability (see Example 2). The following C-terminal derivatives are disclosed in Table C:

TABLE C

| C-terminal Derivatisation | |
|---|---|
| (R² group) | Structure |
| NH-(4-(pyridin-3-yl)butanyl) | 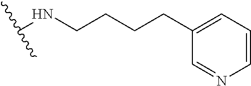 [SMILES: *NCCCCc1cccnc1] |
| NH-(2-(pyridin-3-yl)ethyl) | 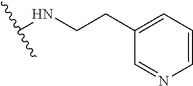 [SMILES: *NCCc1cccnc1] |

For the NH-(4-(pyridin-3-yl)butanyl) and NH-(2-(pyridin-3-yl)ethyl) groups, these are attached to carbonyl carbon of the carboxylic acid group of the C-terminal amino acid residue, such as at Dab. The attachment forms an amide bond.

Lactam A lactam is cyclic amide of formula cyclo(R—NH—C(═O)—R) wherein each R may be any other suitable functional group that joins to the other R. Each R may the same or different.

### Thioether and Dithioether

A thioether is a functional group of the formula R—S—R, wherein R may be any other suitable functional group. A dithioether is a functional group comprising two thioether groups linked together by a linker, such as R—S-L-Y-L-S—R wherein the linker is -L-Y-L-.

### Head-to-Tail Cyclisation

The term “head-to-tail cyclisation” is cyclisation of the N-terminal amine (or derivative thereof) and the C-terminal carboxylic acid to form a cyclic peptide. Typically, this cyclisation forms an amide bond.

### Alkyl

The term “alkyl” refers to a monoradical of a saturated straight or branched hydrocarbon. Preferably, the alkyl group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkyl groups include methyl, ethyl, propyl, iso-propyl (also called 2-propyl or 1 methylethyl), butyl, iso-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, iso-heptyl, n-octyl, 2-ethyl-hexyl, n-nonyl, ndecyl, n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl, n-icosyl, n-triacontyl, n-tetracontyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). In one embodiment, the alkyl is substituted with one or more, such as 1, 2 or 3, such as 1 or 2, such as 1 substituents selected from List A. Examples of a substituted alkyl include chloromethyl, dichloromethyl, fluoromethyl, and difluoromethyl.

### C₁₋₄ Alkyl Groups

C₁₋₄ alkyl groups that may be present as a group R² in the context of compounds of the present invention include, but are not limited to, C₄ alkyl groups such as butyl (n-Bu or —CH₂CH₂CH₂CH₃), or C₁₋₃ alkyl groups, such as methyl (Me or —CH₃, that is a C₁ alkyl group), ethyl (—CH₂CH₃, that is a C₂ alkyl group), 1-propyl (—CH₂CH₂CH₃, that is a C₃ alkyl group), or 2-propyl (—CH(CH₃)₂, that is a C₃ alkyl group).

### C₁₋₃ Alkyl Groups

C₁₋₃ alkyl groups that may be present as a group R² in the context of compounds of the present invention include methyl (Me or —CH₃, that is a C₁ alkyl group), ethyl (—CH₂CH₃, that is a C₂ alkyl group), 1-propyl (—CH₂CH₂CH₃, that is a C₃ alkyl group), and 2-propyl (—CH(CH₃)₂, that is a C₃ alkyl group).

### C₁₋₂ Alkyl Groups

C₁₋₂ alkyl groups that may be present as a group R² in the context of compounds of the present invention include methyl (Me or —CH₃, that is a C₁ alkyl group) and ethyl (—CH₂CH₃, that is a C₂ alkyl group).

### ω-Hydroxy-C₂₋₆ Alkanoic Acid

The residue at the X3 position herein may be a ω-hydroxy-C₂₋₆ alkanoic acid. This residue can be represented by the following structure:

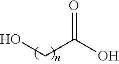

wherein n is 1, 2, 3, 4 or 5.

In some embodiments, n is selected from 1, 2 or 3, such that the ω-hydroxy-C₂₋₆ alkanoic acid is a ω-hydroxy-C₂₋₄ alkanoic acid. In some embodiments, n is 2 such that the ω-hydroxy-C₂₋₆ alkanoic acid is a ω-hydroxy-C₃ alkanoic acid such as 3-hydroxypropanoic acid.

### Alkylene

The term “alkylene” refers to a diradical of a saturated straight or branched hydrocarbon. Preferably, the alkylene group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like.

In one embodiment, alkylene is C₁₋₂₀ alkylene. In one embodiment, alkylene is C₂₋₁₄ alkylene. In one embodiment, alkylene is C₃₋₉ alkylene. In one embodiment, alkylene is C₇₋₉ alkylene.

The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituent may be the same or different). In one embodiment, the alkylene is substituted with one or more, such as 1, 2 or 3, such as 1 or 2, such as 1 substituents selected from List A.

### C₁₋₄ Alkylene Groups

C₁₋₄ alkylene groups that may be present as a group L of the dithioether bridge in the context of compounds of the present invention include, but are not limited to, C₁₋₂ alkylene groups, such as methylene (—CH₂—, that is a C₁ alkylene group) and ethylene (—CH₂CH₂—, that is a C₂ alkylene group).

### Alkyleneoxy

The term “alkyleneoxy”—means “alkylene-O—”, wherein alkylene is defined and exemplified above. In one embodiment, alkyleneoxy means (C₂₋₃)alkyleneoxy. In one embodiment, alkyleneoxy means (C₂)alkyleneoxy (ethyleneoxy). In one embodiment, alkyleneoxy means (C₃)alkyleneoxy (propyleneoxy).

### Carbocyclic and Heterocyclic Groups—Cycloalkyl, Heterocyclyl

The terms “cycloalkyl” or “carbocyclic ring” represents cyclic non-aromatic version of “alkyl” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclobutyl, cyclopentyl, cyclohexyl, cycloheptyl, cyclooctyl, cyclononyl, cyclodecyl, and adamantyl. The cycloalkyl group may consist of one ring (monocyclic), two rings (bicyclic), or more than two rings (polycyclic). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). In one embodiment, the cycloalkyl is substituted with one or more, such as 1, 2 or 3, such as 1 or 2, such as 1 substituent selected from List A.

The term “heterocyclyl” or “heterocyclic ring” means a cycloalkyl group as defined above in which from 1, 2, 3, or 4 carbon atoms in the cycloalkyl group are replaced by heteroatoms of oxygen, nitrogen, silicon, selenium, phosphorus, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the 5 maximum number of S atoms is 1, and the maximum total number of O and S atoms is 2. The term “heterocyclyl” is also meant to encompass partially or completely hydrogenated forms (such as dihydro, tetrahydro or perhydro forms) of the below-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). In one embodiment, the heterocyclyl is substituted with one or more, such as 1, 2 or 3, such as 1 or 2, such as 1 substituents selected from List A.

### Aromatic Groups—Aryl, Heteroaryl

The term “aryl” or “aromatic ring” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, 5 or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). In one embodiment, the aryl is substituted with one or more, such as 1, 2 or 3, such as 1 or 2, such as 1 substituent selected from List A. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-membered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). In one embodiment, the heteroaryl is substituted with one or more, such as 1, 2 or 3, such as 1 or 2, such as 1 substituents selected from List A.

### Optional Substituents

A number of the amino acid residues described herein may be optionally substituted. In some embodiments, the substituent is selected from the group defined by List A below. In some embodiments, the substituent is selected from the group defined by List A1 below. In some embodiments, the substituent is selected from the group defined by List A2 below.

“List A” substituents are selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR′, —N(R′)₂, —S(O)₀₋₂R′, —S(O)₁₋₂OR′, —OS(O)₁₋₂R′, —OS(O)₁₋₂OR′, —S(O)₁₋₂N(R′)₂, —OS(O)₁₋₂N(R′)₂, —N(R′)S(O)₁₋₂R′, —N(R′)S(O)₁₋₂OR′, —C(═X¹)R′, —C(═X¹)X¹R′, —X¹C(═X¹)R′, and —X¹C(═X¹)X¹R′, wherein X¹ is independently selected from O, S, NH and N(CH₃); and each R′ is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃alkyl)₂, —N⁺(C₁₋₃alkyl)₃, —NHS(O)₂(C₁₋₃alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃alkyl)C(═NH)NH_{2-z}(C₁₋₃alkyl)_{z}, wherein each z is independently 0, 1, or 2 and each C₁₋₃ alkyl is independently methyl, ethyl or propyl,

In some embodiments, List A substituents are selected from List A1, consisting of C₁₋₃ alkyl, phenyl, halogen, —CF₃, —OH, —OCH₃, —SCH₃, —NH_{2-z}(CH3)_{z}, —C(═O)OH, C(═O)OCH₃ and —C(═O)CH₃, wherein z is 0, 1, or 2 and C₁₋₃ alkyl is methyl, ethyl, propyl or isopropyl.

In some embodiments, List A substituents are selected from List A2, consisting of C(═O)CH₃, methyl, ethyl, propyl, isopropyl, halogen (such as F, Cl, or Br), and —CF₃.

### Bridging Moieties

The sequences disclosed herein containing bridging moieties noted in the rounded brackets (e.g., (1a), (2a), etc.). These represent chemical bridges between the specific residue pairs. Each rounded bracket will appear twice in the sequence as a pair to indicate a single bridging moiety. Most of the sequences have two bridging moieties, indicated by 4 sets of rounded brackets meaning two bridging moiety pairs.

The number in the rounded bracket indicates a specific bridging moiety pair (e.g. “1” indicates a bridge between amino acid residues at positions 2 and 11, which is also indicated by the square bracket notation defining the specific chemical bridge). The letter indicates the type of chemical bridge (e.g. “a” indicates a 1,3-dithio-propan-2-one bridge).

The specific chemical bridge is defined at the end of the tables, using a square bracket (e.g. [2,11], [4,7], etc.) to indicate the amino acid residues using in the bridging moiety as compared to the original starting peptide (the 13 peptide (isomer 3) as described in Example 2 of WO2023/099669), so these may not line-up specifically with the actual amino acid numbering of the SEQ ID NO: (as in some of these sequences for example, the first amino acid residue had been deleted as compared to the original starting peptide).

The residue directly preceding the rounded bracket notation indicates that specific residue is used in the bridging moiety.

The “*” notation directly after the rounded bracket notation indicates that the terminal —NH₂ (if at the start of the sequence i.e. the N-terminus) or —COOH (if at the end of the sequence i.e. the C-terminus) is used to form the bridge.

### SMILES Strings

The Simplified Molecular-Input Line-Entry System (SMILES) strings are provided below the structures described by the amino acid sequence for each of the compounds disclosed herein. A SMILES string is a line notation for describing the structure of chemical species using short American Standard Code for Information Interchange (ASCII) strings. SMILES strings can be imported by most molecule editors (e.g. ChemDraw®, BIOVIA Draw) for conversion back into two-dimensional or three-dimensional drawings of the chemical structure. Where there is a discrepancy between the structure of the amino acid sequence and the structure provided by the SMILES string, the SMILES string prevails.

### Compounds

The invention provides compounds which are peptide inhibitors of IL-23R. These compounds exhibit a good combination of properties such as highly potent inhibition of IL-23R and/or high stability in the gastrointestinal tract (see Example 3). Furthermore, the compounds described herein may be useful in the treatment of various diseases, conditions and disorders related to IL-23R such as inflammatory bowel disease, such as Crohn's disease or ulcerative colitis, psoriasis and psoriatic arthritis. The compounds described herein exhibit improved properties (such as more potent inhibition and/or higher gastrointestinal tract stability) as compared with the compounds disclosed in WO2023/099669.

The invention provides a compound of the formula:

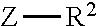

wherein
R² is NHR³ or C(═O)R³, wherein R³ is hydrogen or C₁₋₄ alkyl optionally substituted with a pyridyl ring; and
Z is an amino acid sequence of formula I:

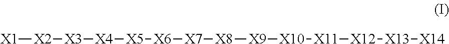

wherein
X1 is absent or is selected from 3-(3-Pyridyl)-Ala or 3-aminopropanoyl;
X2 is selected from the group consisting of 4-aminomethyl-phenylacetyl, an alanine residue substituted by a carbocyclic group or an aromatic or heteroaromatic group selected from the group consisting of phenyl, pyridyl, naphthyl and quinolinyl, each optionally substituted, 3-aminopropanoyl, Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X3 is selected from any amino acid or ω-hydroxy-C₂₋₆ alkanoic acid;
X4 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys;
X5 is selected from the group consisting of an optionally substituted tryptophan residue, an optionally substituted azatryptophan residue, and an optionally substituted beta-homotryptophan residue;
X6 is selected from the group consisting of an optionally substituted Gln residue, an optionally substituted Lys residue, an optionally substituted Arg residue, an optionally substituted Dab residue, an optionally substituted Orn residue, an optionally substituted Phe residue, Ala, D-Ala, beta-Ala, D-beta-Ala, homo-Ala, D-homo-Ala, beta-homo-Ala, N-Me-Ala, N-Me-homo-Ala, Cit, D-Cit, beta-Cit, D-beta-Cit, homo-Cit, D-homo-Cit, beta-homo-Cit, N-Me-Cit, N-Me-homo-Cit, Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Tyr, D-Tyr, beta-Tyr, D-beta-Tyr, homo-Tyr, D-homo-Tyr, beta-homo-Tyr, N-Me-Tyr, N-Me-homo-Tyr, Val, D-Val, beta-Val, D-beta-Val, homo-Val, D-homo-Val, beta-homo-Val, N-Me-Val, N-Me-homo-Val or His, D-His, beta-His, D-beta-His, homo-His, D-homo-His, beta-homo-His, N-Me-His and N-Me-homo-His;
X7 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys;
X8 is selected from the group consisting of an optionally substituted tryptophan residue, an optionally substituted azatryptophan residue, an optionally substituted beta-homotryptophan residue, an optionally substituted tyrosine residue, an optionally substituted phenylalanine residue, an optionally substituted homophenylalanine residue, and an alanine residue substituted by an optionally substituted carbocylic group or an aromatic group selected from the group consisting of phenyl, pyridyl, naphthyl and quinolinyl, each optionally substituted;
X9 is selected from the group consisting of an optionally substituted tryptophan residue, an optionally substituted azatryptophan residue, an optionally substituted beta-homotryptophan residue, an optionally substituted alanine residue, an optionally substituted phenylalanine and an optionally substituted tyrosine residue;
X10 is selected from the group consisting of Val, D-Val, beta-Val, D-beta-Val, homo-Val, D-homo-Val, beta-homo-Val, N-Me-Val, N-Me-homo-Val, 2-Me-Val,
Gly, beta-Gly, homo-Gly, beta-homo-Gly, N-Me-Gly, N-Me-homo-Gly,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, 2-Me-Lys,
Aib, D-Aib, beta-Aib, D-beta-Aib, homo-Aib, D-homo-Aib, beta-homo-Aib, N-Me-Aib, N-Me-homo-Aib,
Ala, D-Ala, beta-Ala, D-beta-Ala, homo-Ala, D-homo-Ala, beta-homo-Ala, N-Me-Ala, N-Me-homo-Ala,
Leu, D-Leu, beta-Leu, D-beta-Leu, homo-Leu, D-homo-Leu, beta-homo-Leu, N-Me-Leu, N-Me-homo-Leu, 2-Me-Leu,
Ile, D-Ile, beta-Ile, D-beta-Ile, homo-Ile, D-homo-Ile, beta-homo-Ile, N-Me-Ile, N-Me-homo-Ile,
and a carbocyclic or heterocyclic ring having an amino substituent and a carbonyl substituent;
X11 is selected from the group consisting of
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, Lys(Me),
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X12 is selected from the group consisting of an optionally substituted Phe residue, an optionally substituted Tyr residue, an optionally substituted His residue, an alanine residue substituted by a carbocyclic group or an aromatic or heteroaromatic group selected from the group consisting of phenyl, pyridyl, naphthyl and quinolinyl, each optionally substituted, Dab, D-Dab, iso-Dab, D-iso-Dab, beta-Dab, D-beta-Dab, homo-Dab, D-homo-Dab, beta-homo-Dab, N-Me-Dab, N-Me-homo-Dab, Gly, beta-Gly, homo-Gly, beta-homo-Gly, N-Me-Gly, N-Me-homo-Gly, Pro, 5-aminopentanoyl, 4-aminopiperidin-4-carbonyl, (R,S)-Imidazolidin-2-carbonyl, 3-aminopropanoyl, Gly-CF3, D-Gly-CF3, Nle, Gln, D-Gln, iso-Gin, D-iso-Gln, beta-Gin, D-beta-Gln, homo-Gin, D-homo-Gln, beta-homo-Gin, N-Me-Gin, N-Me-homo-Gln, THP, Ser, D-Ser, beta-Ser, D-beta-Ser, homo-Ser, D-homo-Ser, beta-homo-Ser, N-Me-Ser, 2-Me-Ser, N-Me-homo-Ser, Ser(OMe), 3-aminotetrahydrofuran-3-carbonyl, THP, Arg, D-Arg, beta-Arg, D-beta-Arg, homo-Arg, D-homo-Arg, beta-homo-Arg, N-Me-Arg, N-Me-homo-Arg, Thr, D-Thr, beta-Thr, D-beta-Thr, homo-Thr, D-homo-Thr, beta-homo-Thr, N-Me-Thr, N-Me-homo-Thr Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Asn, D-Asn, beta-Asn, D-beta-Asn, homo-Asn, D-homo-Asn, beta-homo-Asn, N-Me-Asn, N-Me-homo-Asn, GABA, 2-(trimethyl-2-aminoethoxy)ethoxy]propyl and Lys wherein the side chain —NH₂ of the Lys is substituted with —C(═O)(CH₂)ₙR^{K} wherein n is 0-2 and R^{K} is imidazolyl, pyrimidyl, or pyridyl optionally substituted with F;
X13 is selected from the group consisting of an optionally substituted His residue, an optionally substituted Phe residue, an alanine residue substituted by a carbocyclic group or an aromatic or heteroaromatic group selected from the group consisting of phenyl, pyridyl, naphthyl and quinolinyl, each optionally substituted, Asn, D-Asn, beta-Asn, D-beta-Asn, homo-Asn, D-homo-Asn, beta-homo-Asn, N-Me-Asn, N-Me-homo-Asn, Gly, beta-Gly, homo-Gly, beta-homo-Gly, N-Me-Gly, N-Me-homo-Gly, and Dab, Orn or Lys wherein the side chain —NH₂ is substituted with —C(═O)(CH₂)ₙR^{K} wherein n is 0-2 and R^{K} is imidazolyl, pyrimidyl, or pyridyl optionally substituted with F,
or is absent; and
X14 is absent or is selected from the group consisting of Sar, an optionally substituted His residue, Leu, D-Leu, beta-Leu, D-beta-Leu, homo-Leu, D-homo-Leu, beta-homo-Leu, N-Me-Leu, N-Me-homo-Leu, 2-Me-Leu, and an alanine residue substituted by a carbocyclic group or an aromatic or heteroaromatic group selected from the group consisting of phenyl, pyridyl, naphthyl and quinolinyl, each optionally substituted;
wherein
(i) the amino acid residue at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof;
wherein the compound is not:

| | |
|---|---|
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| Ref 112 | [Dab](1c)*SC(2a)WQC(2a)[Y(2-aminoethoxy)][2-Nal][2-Me- |
| | Leu]E(1c)[{d}R]-[NH2] |
| Ref 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| Ref 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| Ref 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| Ref 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| Ref 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| Ref 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| Ref 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| Ref 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| Ref 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| Ref 130 | [4-Aminomethyl-phenylacetyl](1c)*SE(2c)WQ[Dab](2c)F[2-Nal][2-Me- |
| | Leu]E(1c)[Dab]-[NH2] |
| Ref 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| Ref 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| Ref 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| Ref 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| Ref 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| Ref 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N—Me-Ser]C(2a)WQC(2a)[Y(2- |
| | aminoethoxy)][2-Nal][2-Me-Leu]E(1c)[Dab]-[NH2] |
| Ref 142 | [4-Aminomethyl-phenylacetyl](1c)*S[N—Me-Cys](2a)WQC(2a)[Y(2- |
| | aminoethoxy)][2-Nal][2-Me-Leu]E(1c)[Dab]-[NH2] |
| Ref 143 | [4-Aminomethyl-phenylacetyl](1c)*SC(2a)[N—Me-Trp]QC(2a)[Y(2- |
| | aminoethoxy)][2-Nal][2-Me-Leu]E(1c)[Dab]-[NH2] |
| Ref 144 | [4-Aminomethyl-phenylacetyl](1c)*SC(2a)W[N—Me-Gln]C(2a)[Y(2- |
| | aminoethoxy)][2-Nal][2-Me-Leu]E(1c)[Dab]-[NH2] |
| Ref 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| Ref 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| Ref 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| Ref 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| Ref 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| Ref 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| Ref 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| Ref 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| Ref 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| Ref 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| Ref 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| Ref 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| Ref 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| Ref 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| Ref 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala]-[NH2] |
| Ref 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| Ref 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| Ref 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| Ref 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| Ref 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

Above reference compounds were disclosed in WO2023/099669.

In some embodiments, the invention provides a compound of the formula:

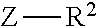

wherein
R² is NHR³ wherein R³ is hydrogen or C₁a alkyl optionally substituted with a pyridyl ring; and
Z is an amino acid sequence of formula I:

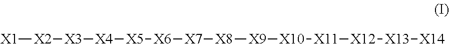

wherein
X1 is absent or is selected from 3-(3-Pyridyl)-Ala or 3-aminopropanoyl;
X2 is selected from the group consisting of 4-aminomethyl-phenylacetyl, 3-(3-Pyridyl)-Ala, and 3-aminopropanoyl;
X3 is selected from the group consisting of Ser, Ala, Phe, and N-Me-Ser;
X4 is Glu or Cys;
X5 is Trp;
X6 is selected from the group consisting of Gln, Q(Me), Q(2Me), Q(pyrrolidin), Ala, Phe, Lys(Ac), Dab(Ac), Cit, and Orn;
X7 is Dab or Cys;
X8 is selected from the group consisting of Y(2-aminoethoxy), homo-Phe, 7-AzaTrp, beta-homo-Trp, and 7-F-Trp;
X9 is selected from the group consisting of 2-Nal, 7-AzaTrp, beta-homo-Trp, and Cyclopropyl-Ala;
X10 is 2-Me-Leu or Aib;
X11 is Glu;
X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser;
X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent; and
X14 is absent or is selected from the group consisting of Sar, D-His, beta-homo-Leu, and 3-(3-Pyridyl)-Ala;
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In all embodiments, the compound of the invention is not:

| | |
|---|---|
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N—Me-Ser]C(2a)WQC(2a)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |

and pharmaceutically acceptable salts an solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

Reference compounds Ref 80, Ref 81, Ref 82, Ref 120, Ref 122, Ref 125, Ref 141, and Ref 146 were disclosed in WO2023/099669.

The present invention is not directed to the compounds disclosed in WO2023/099669.

In some embodiments, the compound is not when X1 is absent; X2 is 4-aminomethyl-phenylacetyl; X3 is Ser or N-Me-Ser; X6 is Gln or Q(pyrrolidin); X8 is Y(2-aminoethoxy); X9 is 2-Nal, X10 is 2-Me-Leu; X12 is Dab, D-Arg, or Ser; X13 is 3-(3-Pyridyl)-Ala or absent; X14 is absent; and R² is NH₂.

In some embodiments, the invention provides a compound of the formula:

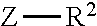

wherein
R² is NHR³ wherein R³ is hydrogen or C₁₋₄ alkyl optionally substituted with a pyridyl ring; and
Z is an amino acid sequence of formula II:

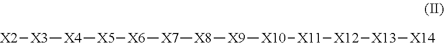

wherein
X2, X3, X4, X5, X6, X7, X8, X9, X10, X11, X12, X13, and X14 are as defined in formula I; and
wherein
(i) the Glu at X11 forms a lactam bridge with the amino acid residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

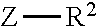

wherein
R² is NHR³ wherein R³ is hydrogen or C₁₋₄ alkyl optionally substituted with a pyridyl ring; and
Z is an amino acid sequence of formula III:

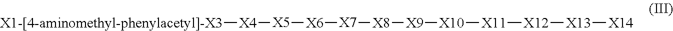

wherein
X1, X3, X4, X5, X6, X7, X8, X9, X10, X11, X12, X13, and X14 are as defined in formula I; and
wherein
(i) the Glu at X11 forms a lactam bridge with the amino acid residue at X1 or the 4-aminomethyl-phenylacetyl residue at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

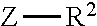

wherein
R² is NHR³ wherein R³ is hydrogen or C₁₋₄ alkyl optionally substituted with a pyridyl ring; and
Z is an amino acid sequence of formula IV:

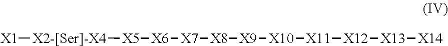

wherein
X1, X2, X4, X5, X6, X7, X8, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

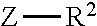

wherein
R² is NHR³ wherein R³ is hydrogen or C₁₋₄ alkyl optionally substituted with a pyridyl ring; and
Z is an amino acid sequence of formula V:

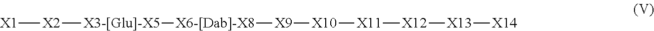

wherein
X1, X2, X3, X5, X6, X8, X9, X10, X11, X12, X13, and X14 are as defined in formula I; and
wherein
(i) the Glu at X11 forms a lactam bridge with the amino acid residue at X1 or at X2 when X1 is absent; and
(ii) the Glu at X4 and the Dab at X7 are amino acid residues who together form a lactam bridge;
or a pharmaceutically acceptable salt or solvate thereof.

In some embodiments, the invention provides a compound of the formula:

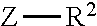

wherein
R² is NHR³ wherein R³ is hydrogen or C₁₋₄ alkyl optionally substituted with a pyridyl ring; and
Z is an amino acid sequence of formula VI:

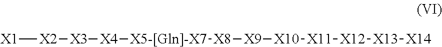

wherein
X1, X2, X3, X4, X5, X7, X8, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

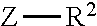

wherein
R² is NHR³ wherein R³ is hydrogen or C₁₋₄ alkyl optionally substituted with a pyridyl ring; and
Z is an amino acid sequence of formula VII:

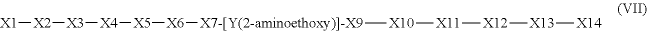

wherein
X1, X2, X3, X4, X5, X6, X7, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

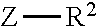

wherein
R² is NHR³ wherein R³ is hydrogen or C₁₋₄ alkyl optionally substituted with a pyridyl ring; and
Z is an amino acid sequence of formula VIII:

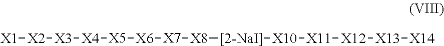

wherein
X1, X2, X3, X4, X5, X6, X7, X8, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

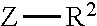

wherein
R² is NHR³ wherein R³ is hydrogen or C₁₋₄ alkyl optionally substituted with a pyridyl ring; and
Z is an amino acid sequence of formula IX:

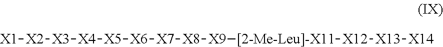

wherein
X1, X2, X3, X4, X5, X6, X7, X8, X9, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

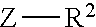

wherein
R² is NHR³ wherein R³ is hydrogen or C₁₋₄ alkyl optionally substituted with a pyridyl ring; and
Z is an amino acid sequence of formula X:

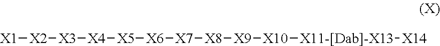

wherein
X1, X2, X3, X4, X5, X6, X7, X8, X9, X10, X11, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

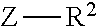

wherein
R² is NHR³ wherein R³ is hydrogen or C₁₋₄ alkyl optionally substituted with a pyridyl ring; and
Z is an amino acid sequence of formula XI:

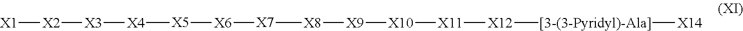

wherein
X1, X2, X3, X4, X5, X6, X7, X8, X9, X10, X11, X12, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

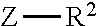

wherein
R² is NHR³ wherein R³ is hydrogen or C₁₋₄ alkyl optionally substituted with a pyridyl ring; and
Z is an amino acid sequence of formula XII:

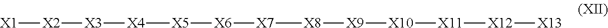

wherein
X1, X2, X3, X4, X5, X6, X7, X8, X9, X10, X11, X12, and X13 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

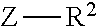

wherein
R² is NHR³ wherein R³ is hydrogen or C₁₋₄ alkyl optionally substituted with a pyridyl ring; and
Z is an amino acid sequence of formula XIII:

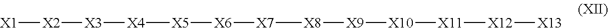

wherein
X2, X3, X4, X5, X6, X7, X8, X9, X10, X11, X12, and X13 are as defined in formula I; and
wherein
(i) the Glu at X11 forms a lactam bridge with the amino acid residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

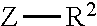

wherein
R² is NHR³ wherein R³ is hydrogen or C₁₋₄ alkyl optionally substituted with a pyridyl ring; and
Z is an amino acid sequence of formula XIV:

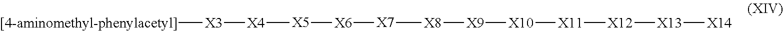

wherein
X3, X4, X5, X6, X7, X8, X9, X10, X11, X12, X13, and X14 are as defined in formula I; and
wherein
(i) the Glu at X11 forms a lactam bridge with the 4-aminomethyl-phenylacetyl residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

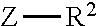

wherein
R² is NHR³ wherein R³ is hydrogen or C₁₋₄ alkyl optionally substituted with a pyridyl ring; and
Z is an amino acid sequence of formula XV:

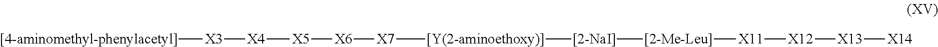

wherein
X3, X4, X5, X6, X7, X11, X12, X13, and X14 are as defined in formula I; and
wherein
(i) the Glu at X11 forms a lactam bridge with the 4-aminomethyl-phenylacetyl residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

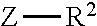

wherein
R² is NHR³ wherein R³ is hydrogen or C₁₋₄ alkyl optionally substituted with a pyridyl ring; and
Z is an amino acid sequence of formula XVI:

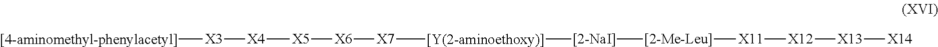

wherein
X3 is selected from the group consisting of Ser or Ala; and
X4, X5, X6, X7, X11, X12, X13, and X14 are as defined in formula I;
wherein
(i) the Glu at X11 forms a lactam bridge with the 4-aminomethyl-phenylacetyl residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

Z—R²
wherein
R² is NHR³ wherein R³ is hydrogen or C₁₋₄ alkyl optionally substituted with a pyridyl ring; and
Z is an amino acid sequence of formula XVII:

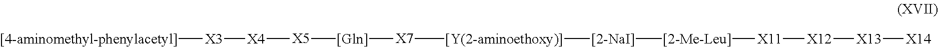

wherein
X3 is selected from the group consisting of Ser or Ala; and
X4, X5, X7, X11, X12, X13, and X14 are as defined in formula I;
wherein
(i) the Glu at X11 forms a lactam bridge with the 4-aminomethyl-phenylacetyl residue at X2; and
(ii) X4 and X7 are amino acid residues who together form a lactam bridge or a dithioether bridge;
or a pharmaceutically acceptable salt or solvate thereof.

In some embodiments, the invention provides a compound of the formula:

Z—R²
wherein
R² is NHR³ wherein R³ is hydrogen or C₁₋₄ alkyl optionally substituted with a pyridyl ring; and
Z is an amino acid sequence of formula XVIII:

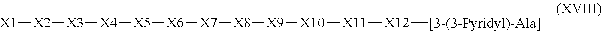

wherein
X1, X2, X3, X4, X5, X6, X7, X8, X9, X10, X11, and X12 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound of the formula:

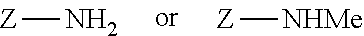

wherein
Z is an amino acid sequence of any one of the formulae I-XVIII as defined above;
or a pharmaceutically acceptable salt or solvate thereof.

In some embodiments, the invention provides a compound of the formula:

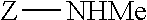

wherein
Z is an amino acid sequence of formula XVIII:

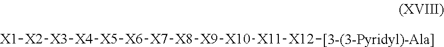

wherein
X1, X2, X3, X4, X5, X6, X7, X8, X9, X10, X11, and X12 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof.

In some embodiments, the invention provides a compound selected from a compound in Table 1-1, or a pharmaceutically acceptable salt or solvate thereof.

In some embodiments, the compound is not:

| | |
|---|---|
| Ref 1 | H-DC(1a)SC(2a)WQC(2a)WW[2-Me-Leu]C(1a)R-[NH2] |
| Ref 2 | H-LC(1a)SC(2a)WQC(2a)WWLC(1a)R-[NH2] |
| Ref 3 | H-DC(1a)SC(2a)WEC(2a)WWLC(1a)R-[NH2] |
| Ref 4 | H-DE(1c)SC(2a)WQC(2a)WWLK(1c)R-[NH2] |
| Ref 5 | H-DC(1a)SE(2c)WQK(2c)WWLC(1a)R-[NH2] |
| Ref 6 | D(1c)*SC(2a)WQC(2a)WWLR(1c)* |
| Ref 7 | [Ac]-DK(1c)SC(2a)WQC(2a)WWLE(1c)R-[NH2] |
| Ref 8 | [Ac]-D[Orn](1c)SC(2a)WQC(2a)WWLE(1c)R-[NH2] |
| Ref 9 | H-DE(1c)SC(2a)WQC(2a)WWL[Dab](1c)R-[NH2] |
| Ref 10 | [Ac]-E(1c)SC(2a)WQC(2a)WWLK(1c)R-[NH2] |
| Ref 11 | [Ac]-DE(1c)SC(2a)WQC(2a)WWLK(1c)-[NH2] |
| Ref 12 | [Ac]-E(1c)SC(2a)WQC(2a)WWLK(1c)-[NH2] |
| Ref 13 | [Ac]-DE(1c)SC(2a)AQC(2a)WWLK(1c)R-[NH2] |
| Ref 14 | [Ac]-DE(1c)SC(2a)WQC(2a)AWLK(1c)R-[NH2] |
| Ref 15 | [Ac]-DE(1c)SC(2a)WQC(2a)WALK(1c)R-[NH2] |
| Ref 16 | [Ac]-DE(1c)SC(2a)[7-Me-Trp]QC(2a)WWLK(1c)R-[NH2] |
| Ref 17 | [Ac]-DE(1c)SC(2a)WQC(2a)W[2-Nal]LK(1c)R-[NH2] |
| Ref 18 | [Ac]-K(1c)SC(2a)WQC(2a)W[2-Nal]LE(1c)R-[NH2] |
| Ref 19 | [Ac]-DK(1c)SC(2a)WQC(2a)W[2-Nal]LE(1c)R-[NH2] |
| Ref 20 | [Ac]-DK(1c)SC(2d)WQC(2d)W[2-Nal]LE(1c)R-[NH2] |
| Ref 21 | [Ac]-DK(1c)SC(2e)WQC(2e)W[2-Nal]LE(1c)R-[NH2] |
| Ref 22 | [Ac]-DK(1c)SC(2f)WQC(2f)W[2-Nal]LE(1c)R-[NH2] |
| Ref 23 | [Ac]-DK(1c)S[K(N3)](2g)WQ[Pra](2g)W[2-Nal]LE(1c)R-[NH2] |
| Ref 24 | [Ac]-DK(1c)S[Aha](2g)WQ[Pra](2g)W[2-Nal]LE(1c)R-[NH2] |
| Ref 25 | [Ac]-K(1c)SC(2a)[2-Nal]QC(2a)W[2-Nal]LE(1c)R-[NH2] |
| Ref 26 | [Ac]-K(1c)SC(2a)[Bip]QC(2a)W[2-Nal]LE(1c)R-[NH2] |
| Ref 27 | [Ac]-K(1c)SC(2a)[1-Me-Trp]QC(2a)W[2-Nal]LE(1c)R-[NH2] |
| Ref 28 | [Ac]-DK(1c)SC(2a)WQC(2a)[2-Nal][2-Nal]LE(1c)R-[NH2] |
| Ref 29 | [Ac]-K(1c)SC(2a)WQC(2a)[Y(2-aminoethoxy)][2-Nal]LE(1c)R-[NH2] |
| Ref 30 | [Ac]-K(1c)SC(2a)WQC(2a)H[2-Nal]LE(1c)R-[NH2] |
| Ref 31 | [Ac]-K(1c)SC(2a)WQC(2a)W[F(4-F)]LE(1c)R-[NH2] |
| Ref 32 | [Ac]-K(1c)SC(2a)WQC(2a)W[1-Me-Trp]LE(1c)R-[NH2] |
| Ref 33 | [Ac]-C(1a)TC(2a)WEC(2a)WW[2-Me-Leu]C(1a)S-[NH2] |
| Ref 34 | [Ac]-GK(1c)TC(2a)WEC(2c)WW[2-Me-Leu]E(1c)S-[NH2] |
| Ref 35 | [Ac]-GC(1a)TC(2a)WEC(2a)W[2-Nal][2-Me-Leu]C(1a)R-[NH2] |
| Ref 36 | [bAla](1c)*SC(2a)WQC(2a)W[2-Nal]LE(1c)R-[NH2] |
| Ref 37 | [(3-Aminomethyl)benzoyl](1c)*SC(2a)WQC(2a)W[2-Nal]LE(1c)R-[NH2] |
| Ref 38 | [(3-Aminomethyl)benzoyl](1c)*SC(2a)WQC(2a)[Y(2-aminoethoxy)][2- |
| | Nal][2-Me-Leu]E(1c)R-[NH2] |
| Ref 39 | [(3-Aminomethyl)benzoyl](1c)*SC(2a)WQC(2a)[Y(2-aminoethoxy)][2- |
| | Nal]LE(1c)R-[NH2] |
| Ref 40 | [(3-Aminomethyl)benzoyl](1c)*SC(2a)WQC(2a)W[2-Nal][2-Me- |
| | Leu]E(1c)R-[NH2] |
| Ref 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| Ref 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| Ref 43 | [(3-Aminomethyl)benzoyl](1c)*SC(2a)WQC(2a)[Y(2-aminoethoxy)][2- |
| | Nal][2-Me-Leu]E(1c)[N—Me-Arg]-[NH2] |
| Ref 44 | [(3-Aminomethyl)benzoyl](1c)*SC(2a)WQC(2a)[Y(2-aminoethoxy)][2- |
| | Nal][2-Me-Leu]E(1c)S-[NH2] |
| Ref 45 | [(3-Aminomethyl)benzoyl](1c)*SC(2a)WQC(2a)[Y(2-aminoethoxy)][2- |
| | Nal][2-Me-Leu]E(1c)[F(4-NH2)]-[NH2] |
| Ref 46 | [(3-Aminomethyl)benzoyl](1c)*SC(2a)WQC(2a)[Y(2-aminoethoxy)][2- |
| | Nal][{d}L]E(1c)Y-[NH2] |
| Ref 47 | [Ac]-K(1c)SC(2a)WQC(2a)[Y(2-aminoethoxy)][2-Nal][2-Me-Leu]E(1c)R- |
| | [NH2] |
| Ref 48 | [Ac]-K(1c)SC(2a)WQC(2a)[Y(2-aminoethoxy)][2-Nal][{d}L]E(1c)R-[NH2] |
| Ref 49 | [(4-Aminomethyl)benzoyl](1c)*SC(2a)WQC(2a)[Y(2-aminoethoxy)][2- |
| | Nal][2-Me-Leu]E(1c)R-[NH2] |
| Ref 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| Ref 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 53 | H-DC(1a)SC(2b)WQC(2b)WWLC(1a)R-[NH2] |
| Ref 54 | [Ac]-K(1c)SC(2b)WQC(2b)[Y(2-aminoethoxy)][2-Nal]LE(1c)R-[NH2] |
| Ref 55 | [Ac]-DE(1c)QC(2a)WQC(2a)YW[2-Me-Leu]K(1c)R-[NH2] |
| Ref 56 | [Ac]-DE(1c)QC(2a)WQC(2a)[2-Nal]W[2-Me-Leu]K(1c)R-[NH2] |
| Ref 57 | [Ac]-DE(1c)TC(2a)WQC(2a)W[2-Nal][2-Me-Leu]K(1c)R-[NH2] |
| Ref 58 | [Ac]-DE(1c)SC(2a)WQC(2a)W[2-Nal][2-Me-Leu]K(1c)ENG-[NH2] |
| Ref 59 | [Ac]-DE(1c)SC(2a)WQC(2a)W[2-Nal][2-Me-Leu]K(1c)NG-[NH2] |
| Ref 60 | [Ac]-DE(1c)SC(2a)WQC(2a)W[2-Nal][2-Me-Leu]K(1c)G-[NH2] |
| Ref 61 | [Ac]-DE(1c)SC(2a)WQC(2a)W[2-Nal][2-Me-Lys]K(1c)ENG-[NH2] |
| Ref 62 | [Ac]-DC(1a)QC(2a)WQC(2a)[2-Nal]W[2-Me-Leu]C(1a)R-[NH2] |
| Ref 63 | [Ac]-DC(1a)QC(2a)WQC(2a)YW[2-Me-Leu]C(1a)R-[NH2] |
| Ref 64 | [Ac]-DC(1a)QC(2a)WQC(2a)WW[2-Me-Leu]C(1a)ENG-[NH2] |
| Ref 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| Ref 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| Ref 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| Ref 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| Ref 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| Ref 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| Ref 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| Ref 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| Ref 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| Ref 74 | [(3-Aminomethyl)benzoyl](1c)*SC(2a)WQC(2a)[Y(2-aminoethoxy)][6-Cl- |
| | Trp][2-Me-Leu]E(1c)R-[NH2] |
| Ref 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| Ref 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| Ref 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| Ref 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| Ref 84 | [Ac]-K(1c)SC(2a)WQC(2a)[Y(2-aminoethoxy)][2-Nal][2-Me- |
| | Leu]E(1c)[{d}R]-[NH2] |
| Ref 85 | [Ac]-K(1c)TC(2a)WQC(2a)[Y(2-aminoethoxy)][2-Nal][2-Me- |
| | Leu]E(1c)[{d}R]-[NH2] |
| Ref 86 | [Ac]-K(1c)SC(2a)WQC(2a)[Y(2-aminoethoxy)][2-Nal][2-Me-Leu]E(1c)S- |
| | [NH2] |
| Ref 87 | [6-Aminohexanoyl](1c)*SC(2a)WQC(2a)[Y(2-aminoethoxy)][2-Nal][2-Me- |
| | Leu]E(1c)[{d}R]-[NH2] |
| Ref 88 | [6-Amino-4-oxahexanoyl](1c)*SC(2a)WQC(2a)[Y(2-aminoethoxy)][2- |
| | Nal][2-Me-Leu]E(1c)[{d}R]-[NH2] |
| Ref 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| Ref 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| Ref 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| Ref 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 94 | [Ac]-[K(N3)](1g)SC(2a)WQC(2a)[Y(2-aminoethoxy)][2-Nal][2-Me- |
| | Leu][Pra](1g)[{d}R]-[NH2] |
| Ref 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| Ref 96 | [Azidoacetic acid](1g)*SC(2a)WQC(2a)[Y(2-aminoethoxy)][2-Nal][2-Me- |
| | Leu][Dab(3-butynoic acid)](1g)[{d}R]-[NH2] |
| Ref 97 | [but-3-ynoic acid](1g)*SC(2a)WQC(2a)[Y(2-aminoethoxy)][2-Nal][2-Me- |
| | Leu][Dab(azidoacetic acid)](1g)[{d}R]-[NH2] |
| Ref 98 | [(N3)-Ala](1g)*SC(2a)WQC(2a)[Y(2-aminoethoxy)][2-Nal][2-Me- |
| | Leu][Glu(propargylamine)](1g)[{d}R]-[NH2] |
| Ref 99 | [(N3)-Ala](1g)*SC(2a)WQC(2a)[Y(2-aminoethoxy)][2-Nal][2-Me- |
| | Leu][Dab(3-butynoic acid)](1g)[{d}R]-[NH2] |
| Ref 100 | [but-3-ynoic acid](1g)*SC(2a)WQC(2a)[Y(2-aminoethoxy)][2-Nal][2-Me- |
| | Leu][Dab((N3)-Ala))](1g)[{d}R]-[NH2] |
| Ref 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| Ref 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| Ref 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| Ref 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R]-[NH2] |
| Ref 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |
| Ref 106 | [but-3-ynoic acid](1h)*SC(2a)WQC(2a)[Y(2-aminoethoxy)][2-Nal][2-Me- |
| | Leu][Dab(azidoacetic acid)](1h)[{d}R]-[NH2] |
| Ref 107 | [but-3-ynoic acid](1h)*SC(2a)WQC(2a)[Y(2-aminoethoxy)][2-Nal][2-Me- |
| | Leu][Dab((N3)-Ala))](1h)[{d}R]-[NH2] |
| Ref 108 | [4-Aminomethyl-phenylacetyl](1c)*SC(2a)WQC(2a)[Y(2-aminoethoxy)][2- |
| | Nal][2-Me-Leu]E(1c)[{d}R]-[OH] |
| Ref 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)]-[NH2] |
| Ref 110 | [4-Aminomethyl-phenylacetyl](1c)*SC(2a)WQC(2a)[Dab][2-Nal][2-Me- |
| | Leu]E(1c)[{d}R]-[NH2] |
| Ref 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R]-[NH2] |
| Ref 112 | [Dab](1c)*SC(2a)WQC(2a)[Y(2-aminoethoxy)][2-Nal][2-Me- |
| | Leu]E(1c)[{d}R]-[NH2] |
| Ref 113 | [2,4-Diaminobutanoyl([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R]-[NH2] |
| Ref 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| Ref 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| Ref 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| Ref 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| Ref 118 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[OH] |
| Ref 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| Ref 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| Ref 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| Ref 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| Ref 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| Ref 130 | [4-Aminomethyl-phenylacetyl](1c)*SE(2c)WQ[Dab](2c)F[2-Nal][2-Me- |
| | Leu]E(1c)[Dab]-[NH2] |
| Ref 131 | [4-Aminomethyl-phenylacetyl](1c)*SE(2c)WQ[Dab](2c)V[2-Nal][2-Me- |
| | Leu]E(1c)[Dab]-[NH2] |
| Ref 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| Ref 133 | [4-Aminomethyl-phenylacetyl](1c)*SE(2c)WQ[Dab](2c)[Y(2- |
| | aminoethoxy)]H[2-Me-Leu]E(1c)[Dab]-[NH2] |
| Ref 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| Ref 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| Ref 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| Ref 137 | [4-Aminomethyl-phenylacetyl](1c)*SE(2c)WQ[Dab](2c)[Y(2- |
| | aminoethoxy)][2-Nal][2-Me-Leu]E(1c)-[NH-3-aminopropanoyl] |
| Ref 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| Ref 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| Ref 140 | [4-Methylaminomethyl-phenylacetyl](1c)*SC(2a)WQC(2a)[Y(2- |
| | aminoethoxy)][2-Nal][2-Me-Leu]E(1c)[Dab]-[NH2] |
| Ref 141 | [4-Aminomethyl-phenylacetyl](1c)*[N—Me-Ser]C(2a)WQC(2a)[Y(2- |
| | aminoethoxy)][2-Nal][2-Me-Leu]E(1c)[Dab]-[NH2] |
| Ref 142 | [4-Aminomethyl-phenylacetyl](1c)*S[N—Me-Cys](2a)WQC(2a)[Y(2- |
| | aminoethoxy)][2-Nal][2-Me-Leu]E(1c)[Dab]-[NH2] |
| Ref 143 | [4-Aminomethyl-phenylacetyl](1c)*SC(2a)[N—Me-Trp]QC(2a)[Y(2- |
| | aminoethoxy)][2-Nal][2-Me-Leu]E(1c)[Dab]-[NH2] |
| Ref 144 | [4-Aminomethyl-phenylacetyl](1c)*SC(2a)W[N—Me-Gln]C(2a)[Y(2- |
| | aminoethoxy)][2-Nal][2-Me-Leu]E(1c)[Dab]-[NH2] |
| Ref 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala]-[NH2] |
| Ref 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]]-[NH2] |
| Ref 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl]-[NH2] |
| Ref 152 | [(3-Aminomethyl)benzoyl](1c)*SC(2a)WQC(2a)][Y(2-aminoethoxy)][2- |
| | Nal][2-Me-Leu]E(1c)R[F(3-F)]-[NH2] |
| Ref 153 | [(3-Aminomethyl)benzoyl](1c)*SC(2a)WQC(2a)][Y(2-aminoethoxy)][2- |
| | Nal][2-Me-Leu]E(1c)R[F(3,5-F)]-[NH2] |
| Ref 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl]-[NH2] |
| Ref 155 | [Ac]-K(1c)TC(2a)WQC(2a)[Y(2-aminoethoxy)][2-Nal][2-Me- |
| | Leu]E(1c)[Dab][3-(3-Pyridyl)-Ala]-[NH2] |
| Ref 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala]-[NH2] |
| Ref 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| Ref 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| Ref 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| Ref 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| Ref 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| Ref 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| Ref 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| Ref 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| Ref 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| Ref 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| Ref 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| Ref 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| Ref 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| Ref 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| Ref 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala]-[NH2] |
| Ref 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| Ref 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| Ref 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala]-[NH2] |
| Ref 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| Ref 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| Ref 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

Any internal truncation, that is deletion of amino acid residues, between X2 and X11 leads to inactive compounds (see reference compounds Ref 5, Ref 6, and Ref 7 in the Examples of WO2023/099669).

It will be understood that the invention encompasses salts and solvates of the compounds. Suitable salts and solvates of peptides are known in the art.

It will also be understood any of the following references to embodiments may be applicable and are combinable with any of the formulae described herein.

### R²

R² is NHR³ or C(═O)R³, wherein R³ is hydrogen or C₁₋₄ alkyl optionally substituted with a pyridyl ring.

In some embodiments, R² is NHR³ wherein R³ is hydrogen or C₁₋₄ alkyl optionally substituted with a pyridyl ring.

In some embodiments, R² is NHR³ wherein R³ is hydrogen or C₁₋₄ alkyl optionally substituted with a pyrid-3-yl ring.

In some embodiments, R² is NHR³ wherein R³ is hydrogen or C₁₋₄ alkyl. In some embodiments, R² is NHR³ wherein R³ is hydrogen or C₁₋₃ alkyl. In some embodiments, R² is NHR³ wherein R³ is hydrogen or C₁₋₂ alkyl. In some embodiments, R² is NHR³ wherein R³ is hydrogen or C₁ alkyl (methyl or Me).

In some embodiments, R² is NHR³ wherein R³ is C₁₋₄ alkyl optionally substituted with a pyridyl ring. In some embodiments, R² is NHR³ wherein R³ is C₁₋₄ alkyl substituted with a pyridyl ring.

In some embodiments, R² is NHR³ wherein R³ is C₁₋₄ alkyl optionally substituted with a pyrid-3-yl ring. In some embodiments, R² is NHR³ wherein R³ is C₁₋₄ alkyl substituted with a pyrid-3-yl ring.

In some embodiments, R² is NHR³ wherein R³ is C₄ alkyl substituted with a pyridyl ring. In some embodiments, R² is NHR³ wherein R³ is C₄ alkyl substituted with a pyrid-3-yl ring. In some embodiments, R² is NHR³ wherein R³ is n-Bu substituted with a pyrid-3-yl ring. In some embodiments, R² is NHR³ wherein R³ is —CH₂CH₂CH₂CH₂(pyrid-3-yl). That is, NHCH₂CH₂CH₂CH₂(pyrid-3-yl) or NH-(4-(pyridin-3-yl)butanyl). The group NH-(4-(pyridin-3-yl)butanyl) has the structure:

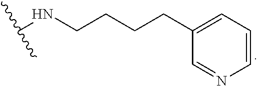 [SMILES: *NCCCCc1cccnc1]

In some embodiments, R² is NHR³ wherein R³ is C₂ alkyl substituted with a pyridyl ring. In some embodiments, R² is NHR³ wherein R³ is C₂ alkyl substituted with a pyrid-3-yl ring. In some embodiments, R² is NHR³ wherein R³ is ethyl (Et) substituted with a pyrid-3-yl ring. In some embodiments, R² is NHR³ wherein R³ is —CH₂CH₂(pyrid-3-yl). That is, NHCH₂CH₂(pyrid-3-yl) or NH-(2-(pyridin-3-yl)ethyl). The group NH-(2-(pyridin-3-yl)ethyl) has the structure:

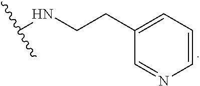 [SMILES: *NCCc1cccnc1]

Preferably in embodiments where R² is NHR³ wherein R³ is C₁₋₄ alkyl substituted with a pyridyl ring, such as NH-(4-(pyridin-3-yl)butanyl) and NH-(2-(pyridin-3-yl)ethyl), both X13 and X14 are absent.

In some embodiments, R² is NHMe, NH₂, NHCH₂CH₂CH₂CH₂(pyrid-3-yl) [that is, NH-(4-(pyridin-3-yl)butanyl)], or NHCH₂CH₂(pyrid-3-yl) [that is, NH-(2-(pyridin-3-yl)ethyl)].

In some embodiments, R² is NHMe. In some embodiments, R² is NH₂. In some embodiments, R² is NHCH₂CH₂CH₂CH₂(pyrid-3-yl), that is NH-(4-(pyridin-3-yl)butanyl). In some embodiments, R² is NHCH₂CH₂(pyrid-3-yl), that is NH-(2-(pyridin-3-yl)ethyl).

Preferably, R² is NHMe or NH₂. Even more preferably, R² is NHMe.

In particular, R² is NHMe is preferable because the modification improves SIF stability (see Example 2).

### Z

Z is an amino acid sequence of the formula:

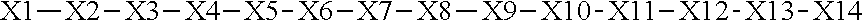

wherein
X1 is absent or is selected from 3-(3-Pyridyl)-Ala or 3-aminopropanoyl;
X2 is selected from the group consisting of 4-aminomethyl-phenylacetyl, an alanine residue substituted by a carbocyclic group or an aromatic or heteroaromatic group selected from the group consisting of phenyl, pyridyl, naphthyl and quinolinyl, each optionally substituted, 3-aminopropanoyl, Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X3 is selected from any amino acid or ω-hydroxy-C₂₋₆ alkanoic acid;
X4 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys;
X5 is selected from the group consisting of an optionally substituted tryptophan residue, an optionally substituted azatryptophan residue, and an optionally substituted beta-homotryptophan residue;
X6 is selected from the group consisting of of an optionally substituted Gln residue, an optionally substituted Lys residue, an optionally substituted Arg residue, an optionally substituted Dab residue, an optionally substituted Orn residue, an optionally substituted Phe residue, Ala, D-Ala, beta-Ala, D-beta-Ala, homo-Ala, D-homo-Ala, beta-homo-Ala, N-Me-Ala, N-Me-homo-Ala, Cit, D-Cit, beta-Cit, D-beta-Cit, homo-Cit, D-homo-Cit, beta-homo-Cit, N-Me-Cit, N-Me-homo-Cit, Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Tyr, D-Tyr, beta-Tyr, D-beta-Tyr, homo-Tyr, D-homo-Tyr, beta-homo-Tyr, N-Me-Tyr, N-Me-homo-Tyr, Val, D-Val, beta-Val, D-beta-Val, homo-Val, D-homo-Val, beta-homo-Val, N-Me-Val, N-Me-homo-Val or His, D-His, beta-His, D-beta-His, homo-His, D-homo-His, beta-homo-His, N-Me-His and N-Me-homo-His;
X7 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys;
X8 is selected from the group consisting of an optionally substituted tryptophan residue, an optionally substituted azatryptophan residue, an optionally substituted beta-homotryptophan residue, an optionally substituted tyrosine residue, an optionally substituted phenylalanine residue, an optionally substituted homophenylalanine residue, and an alanine residue substituted by an optionally substituted carbocylic group or an aromatic group selected from the group consisting of phenyl, pyridyl, naphthyl and quinolinyl, each optionally substituted;
X9 is selected from the group consisting of an optionally substituted tryptophan residue, an optionally substituted azatryptophan residue, an optionally substituted beta-homotryptophan residue, an optionally substituted alanine residue, an optionally substituted phenylalanine and an optionally substituted tyrosine residue;
X10 is selected from the group consisting of Val, D-Val, beta-Val, D-beta-Val, homo-Val, D-homo-Val, beta-homo-Val, N-Me-Val, N-Me-homo-Val, 2-Me-Val,
Gly, beta-Gly, homo-Gly, beta-homo-Gly, N-Me-Gly, N-Me-homo-Gly,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, 2-Me-Lys,
Aib, D-Aib, beta-Aib, D-beta-Aib, homo-Aib, D-homo-Aib, beta-homo-Aib, N-Me-Aib, N-Me-homo-Aib,
Ala, D-Ala, beta-Ala, D-beta-Ala, homo-Ala, D-homo-Ala, beta-homo-Ala, N-Me-Ala, N-Me-homo-Ala,
Leu, D-Leu, beta-Leu, D-beta-Leu, homo-Leu, D-homo-Leu, beta-homo-Leu, N-Me-Leu, N-Me-homo-Leu, 2-Me-Leu,
Ile, D-Ile, beta-Ile, D-beta-Ile, homo-Ile, D-homo-Ile, beta-homo-Ile, N-Me-Ile, N-Me-homo-Ile,
and a carbocyclic or heterocyclic ring having an amino substituent and a carbonyl substituent;
X11 is selected from the group consisting of Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, Lys(Me),
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X12 is selected from the group consisting of an optionally substituted Phe residue, an optionally substituted Tyr residue, an optionally substituted His residue, an alanine residue substituted by a carbocyclic group or an aromatic or heteroaromatic group selected from the group consisting of phenyl, pyridyl, naphthyl and quinolinyl, each optionally substituted, Dab, D-Dab, iso-Dab, D-iso-Dab, beta-Dab, D-beta-Dab, homo-Dab, D-homo-Dab, beta-homo-Dab, N-Me-Dab, N-Me-homo-Dab, Gly, beta-Gly, homo-Gly, beta-homo-Gly, N-Me-Gly, N-Me-homo-Gly, Pro, 5-aminopentanoyl, 4-aminopiperidin-4-carbonyl, (R,S)-Imidazolidin-2-carbonyl, 3-aminopropanoyl, Gly-CF3, D-Gly-CF3, Nle, Gln, D-Gln, iso-Gln, D-iso-Gln, beta-Gin, D-beta-Gln, homo-Gln, D-homo-Gln, beta-homo-Gln, N-Me-Gln, N-Me-homo-Gln, THP, Ser, D-Ser, beta-Ser, D-beta-Ser, homo-Ser, D-homo-Ser, beta-homo-Ser, N-Me-Ser, 2-Me-Ser, N-Me-homo-Ser, Ser(OMe), 3-aminotetrahydrofuran-3-carbonyl, THP, Arg, D-Arg, beta-Arg, D-beta-Arg, homo-Arg, D-homo-Arg, beta-homo-Arg, N-Me-Arg, N-Me-homo-Arg, Thr, D-Thr, beta-Thr, D-beta-Thr, homo-Thr, D-homo-Thr, beta-homo-Thr, N-Me-Thr, N-Me-homo-Thr Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Asn, D-Asn, beta-Asn, D-beta-Asn, homo-Asn, D-homo-Asn, beta-homo-Asn, N-Me-Asn, N-Me-homo-Asn, GABA, 2-(trimethyl-2-aminoethoxy)ethoxy]propyl and Lys wherein the side chain —NH₂ of the Lys is substituted with —C(═O)(CH₂)ₙR^{K} wherein n is 0-2 and R^{K} is imidazolyl, pyrimidyl, or pyridyl optionally substituted with F;
X13 is selected from the group consisting of an optionally substituted His residue, an optionally substituted Phe residue, an alanine residue substituted by a carbocyclic group or an aromatic or heteroaromatic group selected from the group consisting of phenyl, pyridyl, naphthyl and quinolinyl, each optionally substituted, Asn, D-Asn, beta-Asn, D-beta-Asn, homo-Asn, D-homo-Asn, beta-homo-Asn, N-Me-Asn, N-Me-homo-Asn, Gly, homo-Gly, beta-homo-Gly, N-Me-Gly, N-Me-homo-Gly, and Dab, Orn or Lys wherein the side chain —NH₂ is substituted with —C(═O)(CH₂)ₙR^{K} wherein n is 0-2 and R^{K} is imidazolyl, pyrimidyl, or pyridyl optionally substituted with F, or is absent; and
X14 is absent or is selected from the group consisting of Sar, an optionally substituted His residue, Leu, D-Leu, beta-Leu, D-beta-Leu, homo-Leu, D-homo-Leu, beta-homo-Leu, N-Me-Leu, N-Me-homo-Leu, 2-Me-Leu, and an alanine residue substituted by a carbocyclic group or an aromatic or heteroaromatic group selected from the group consisting of phenyl, pyridyl, naphthyl and quinolinyl, each optionally substituted;
wherein
(i) the amino acid residue at X1 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof;
wherein the compound is not:

| | |
|---|---|
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| Ref 112 | [Dab](1c)*SC(2a)WQC(2a)[Y(2-aminoethoxy)][2-Nal][2-Me- |
| | Leu]E(1c)[{d}R]-[NH2] |
| Ref 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| Ref 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| Ref 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| Ref 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| Ref 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| Ref 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| Ref 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| Ref 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| Ref 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| Ref 130 | [4-Aminomethyl-phenylacetyl](1c)*SE(2c)WQ[Dab](2c)F[2-Nal][2-Me- |
| | Leu]E(1c)[Dab]-[NH2] |
| Ref 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| Ref 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| Ref 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| Ref 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| Ref 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| Ref 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N—Me-Ser]C(2a)WQC(2a)[Y(2- |
| | aminoethoxy)][2-Nal][2-Me-Leu]E(1c)[Dab]-[NH2] |
| Ref 142 | [4-Aminomethyl-phenylacetyl](1c)*S[N—Me-Cys](2a)WQC(2a)[Y(2- |
| | aminoethoxy)][2-Nal][2-Me-Leu]E(1c)[Dab]-[NH2] |
| Ref 143 | [4-Aminomethyl-phenylacetyl](1c)*SC(2a)[N—Me-Trp]QC(2a)[Y(2- |
| | aminoethoxy)][2-Nal][2-Me-Leu]E(1c)[Dab]-[NH2] |
| Ref 144 | [4-Aminomethyl-phenylacetyl](1c)*SC(2a)W[N—Me-Gln]C(2a)[Y(2- |
| | aminoethoxy)][2-Nal][2-Me-Leu]E(1c)[Dab]-[NH2] |
| Ref 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| Ref 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| Ref 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| Ref 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| Ref 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| Ref 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| Ref 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| Ref 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| Ref 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| Ref 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| Ref 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| Ref 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| Ref 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| Ref 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| Ref 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala]-[NH2] |
| Ref 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| Ref 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| Ref 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| Ref 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| Ref 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

In some embodiments, Z is an amino acid sequence of formula I:

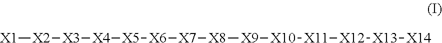

wherein
X1 is absent or is selected from 3-(3-Pyridyl)-Ala or 3-aminopropanoyl;
X2 is selected from the group consisting of 4-aminomethyl-phenylacetyl, 3-(3-Pyridyl)-Ala, and 3-aminopropanoyl;
X3 is selected from the group consisting of Ser, Ala, Phe, and N-Me-Ser;
X4 is Glu or Cys;
X5 is Trp;
X6 is selected from the group consisting of Gln, Q(Me), Q(2Me), Q(pyrrolidin), Ala, Phe, Lys(Ac), Dab(Ac), Cit, and Orn;
X7 is Dab or Cys;
X8 is selected from the group consisting of Y(2-aminoethoxy), homo-Phe, 7-AzaTrp, beta-homo-Trp, and 7-F-Trp;
X9 is selected from the group consisting of 2-Nal, 7-AzaTrp, beta-homo-Trp, and Cyclopropyl-Ala;
X10 is 2-Me-Leu or Aib;
X11 is Glu;
X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser;
X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent; and
X14 is absent or is selected from the group consisting of Sar, D-His, beta-homo-Leu, and 3-(3-Pyridyl)-Ala;
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula II:

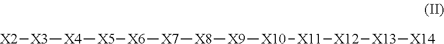

wherein
X2, X3, X4, X5, X6, X7, X8, X9, X10, X11, X12, X13, and X14 are as defined in formula I; and
wherein
(i) the Glu at X11 forms a lactam bridge with the amino acid residue at X2; and
(ii) X4 and X7 are amino acid residues who together form a lactam bridge or a dithioether bridge.

In some embodiments, Z is an amino acid sequence of formula III:

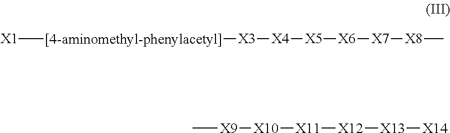

wherein
X1, X3, X4, X5, X6, X7, X8, X9, X10, X11, X12, X13, and X14 are as defined in formula I; and
wherein
(i) the Glu at X11 forms a lactam bridge with the amino acid residue at X1 or the 4-aminomethyl-phenylacetyl residue at X2 when X1 is absent; and
(ii) X4 and X7 are amino acid residues who together form a lactam bridge or a dithioether bridge.

In some embodiments, Z is an amino acid sequence of formula IV:

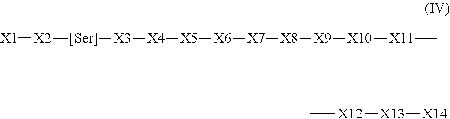

wherein
X1, X2, X4, X5, X6, X7, X8, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula V:

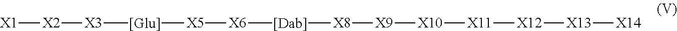

wherein
X1, X2, X3, X5, X6, X8, X9, X10, X11, X12, X13, and X14 are as defined in formula I; and
wherein
(i) the Glu at X11 forms a lactam bridge with the amino acid residue at X1 or at X2 when X1 is absent; and
(ii) the Glu at X4 and the Dab at X7 are amino acid residues who together form a lactam bridge.

In some embodiments, Z is an amino acid sequence of formula VI:

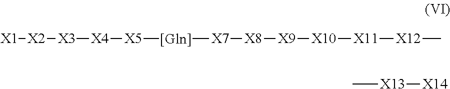

wherein
X1, X2, X3, X4, X5, X7, X8, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula VII:

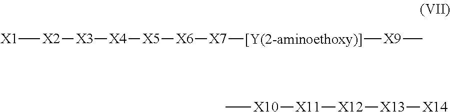

wherein
X1, X2, X3, X4, X5, X6, X7, X9, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula VIII:

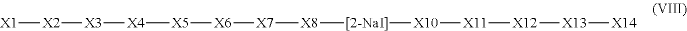

wherein
X1, X2, X3, X4, X5, X6, X7, X8, X10, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula IX:

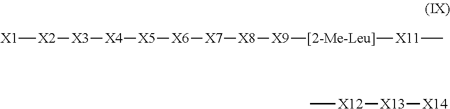

wherein
X1, X2, X3, X4, X5, X6, X7, X8, X9, X11, X12, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula X:

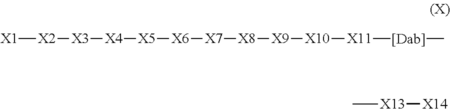

wherein
X1, X2, X3, X4, X5, X6, X7, X8, X9, X10, X11, X13, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula XI:

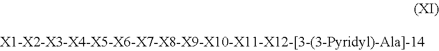

wherein
X1, X2, X3, X4, X5, X6, X7, X8, X9, X10, X11, X12, and X14 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula XII:

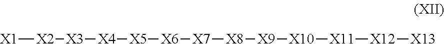

wherein
X1, X2, X3, X4, X5, X6, X7, X8, X9, X10, X11, X12, and X13 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence of formula XIII:

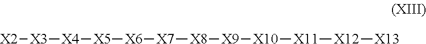

wherein
X2, X3, X4, X5, X6, X7, X8, X9, X10, X11, X12, and X13 are as defined in formula I; and
wherein
(i) the Glu at X11 forms a lactam bridge with the amino acid residue at X2; and
(ii) X4 and X7 are amino acid residues who together form a lactam bridge or a dithioether bridge.

In some embodiments, Z is an amino acid sequence of formula XIV:

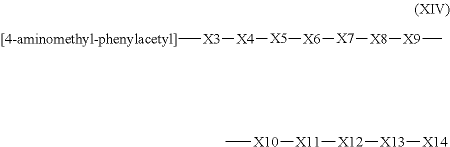

wherein
X3, X4, X5, X6, X7, X8, X9, X10, X11, X12, X13, and X14 are as defined in formula I; and
wherein
(i) the Glu at X11 forms a lactam bridge with the 4-aminomethyl-phenylacetyl residue at X2; and
(ii) X4 and X7 are amino acid residues who together form a lactam bridge or a dithioether bridge.

In some embodiments, Z is an amino acid sequence of formula XV:

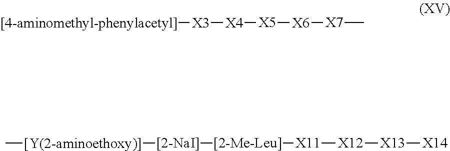

wherein
X3, X4, X5, X6, X7, X11, X12, X13, and X14 are as defined in formula I; and
wherein
(i) the Glu at X11 forms a lactam bridge with the 4-aminomethyl-phenylacetyl residue at X2; and
(ii) X4 and X7 are amino acid residues who together form a lactam bridge or a dithioether bridge.

In some embodiments, Z is an amino acid sequence of formula XVI:

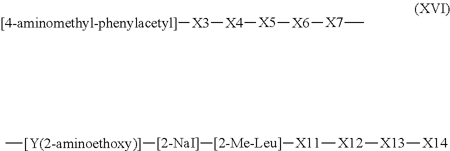

wherein
X3 is selected from the group consisting of Ser or Ala; and
X4, X5, X6, X7, X11, X12, X13, and X14 are as defined in formula I;
wherein
(i) the Glu at X11 forms a lactam bridge with the 4-aminomethyl-phenylacetyl residue at X2; and
(ii) X4 and X7 are amino acid residues who together form a lactam bridge or a dithioether bridge.

In some embodiments, Z is an amino acid sequence of formula XVII:

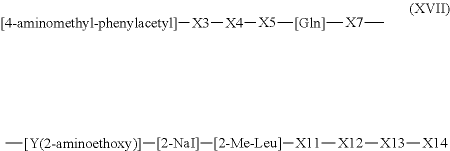

wherein
X3 is selected from the group consisting of Ser or Ala; and
X4, X5, X7, X11, X12, X13, and X14 are as defined in formula I;
wherein
(i) the Glu at X11 forms a lactam bridge with the 4-aminomethyl-phenylacetyl residue at X2; and
(ii) X4 and X7 are amino acid residues who together form a lactam bridge or a dithioether bridge.

In some embodiments, Z is an amino acid sequence of formula XVIII:

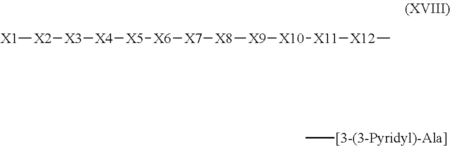

wherein
X1, X2, X3, X4, X5, X6, X7, X8, X9, X10, X11, and X12 are as defined in formula I; and
wherein
(i) the Glu at X11 forms a lactam bridge with the amino acid residue at X1 or at X2 when X1 is absent; and
(ii) X4 and X7 are amino acid residues who together form a lactam bridge or a dithioether bridge.

In some embodiments, Z is an amino acid sequence selected from the group consisting of a sequence listed in Table 1-1a.

### X1

X1 is absent or is selected from the group consisting of 3-(3-Pyridyl)-Ala or 3-aminopropanoyl.

In some embodiments, X1 is absent. When X1 is absent, the amino acid residue at X2 forms a lactam bridge with the Glu residue at X11.

In some embodiments, X1 is 3-(3-Pyridyl)-Ala or 3-aminopropanoyl. In some embodiments, X1 is 3-(3-Pyridyl)-Ala. In some embodiments, X1 is 3-aminopropanoyl.

When X1 is present, that is when X1 is 3-(3-Pyridyl)-Ala or 3-aminopropanoyl, X1 may form a lactam bridge with the Gly residue at X11. In some embodiments when X1 is present, that is when X1 is 3-(3-Pyridyl)-Ala or 3-aminopropanoyl, X1 forms a lactam bridge with the Glu residue at X11.

Preferably, X1 is absent.

### X2

X2 is selected from the group consisting of 4-aminomethyl-phenylacetyl, an alanine residue substituted by a carbocyclic group or an aromatic or heteroaromatic group selected from the group consisting of phenyl, pyridyl, naphthyl and quinolinyl, each optionally substituted, 3-aminopropanoyl, Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl.

In some embodiments, X2 is selected from the group consisting of 4-aminomethyl-phenylacetyl, 3-(3-Pyridyl)-Ala, and 3-aminopropanoyl.

The amino acid residue at X2 may form a lactam bridge with the Glu residue at X11. When X1 is absent, the amino acid residue at X2 forms a lactam bridge with the Glu residue at X11.

In some embodiments, X2 is 4-aminomethyl-phenylacetyl. In some embodiments, X2 is 3-(3-Pyridyl)-Ala. In some embodiments, X2 is 3-aminopropanoyl.

Preferably, X2 is 4-aminomethyl-phenylacetyl.

### X3

X3 is selected from any amino acid or ω-hydroxy-C₂₋₆ alkanoic acid.

In some embodiments, X3 is selected from the group consisting of Ser, Ala, N-Me-Ala, Phe, and N-Me-Ser.

In some embodiments, X3 is selected from the group consisting of Ser, Ala, Phe, and N-Me-Ser.

In some embodiments, X3 is Ser or Ala.

In some embodiments, X3 is Ser. In some embodiments, X3 is Ala. In some embodiments, X3 is Phe. In some embodiments, X3 is N-Me-Ser.

Preferably, X3 is Ser.

### X4

X4 is selected from Glu D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys.

In some embodiments, X4 is Glu or Cys. The amino acid residue at X4 forms a lactam bridge or a dithioether bridge with the amino acid residue at X7.

In some embodiments, X4 is Glu. When X4 is Glu, the Glu at X4 forms a lactam bridge with the amino acid residue at X7.

In some embodiments, X4 is Cys. When X4 is Cys, the Cys at X4 forms a dithioether bridge with the amino acid residue at X7.

### Preferably, X4 is Glu.

### X5

X5 is selected from the group consisting of an optionally substituted tryptophan residue, an optionally substituted azatryptophan residue, and an optionally substituted beta-homotryptophan residue.

In some embodiments, X5 is Trp or 7-Me-Trp.

In some embodiments, X5 is Trp.

### X6

X6 is selected from the group consisting of an optionally substituted Gln residue, an optionally substituted Lys residue, an optionally substituted Arg residue, an optionally substituted Dab residue, an optionally substituted Orn residue, an optionally substituted Phe residue, Ala, D-Ala, beta-Ala, D-beta-Ala, homo-Ala, D-homo-Ala, beta-homo-Ala, N-Me-Ala, N-Me-homo-Ala, Cit, D-Cit, beta-Cit, D-beta-Cit, homo-Cit, D-homo-Cit, beta-homo-Cit, N-Me-Cit, N-Me-homo-Cit, Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Tyr, D-Tyr, beta-Tyr, D-beta-Tyr, homo-Tyr, D-homo-Tyr, beta-homo-Tyr, N-Me-Tyr, N-Me-homo-Tyr, Val, D-Val, beta-Val, D-beta-Val, homo-Val, D-homo-Val, beta-homo-Val, N-Me-Val, N-Me-homo-Val or His, D-His, beta-His, D-beta-His, homo-His, D-homo-His, beta-homo-His, N-Me-His and N-Me-homo-His.

In some embodiments, X6 is selected from the group consisting of Gln, Q(Me), Q(2Me), Q(pyrrolidin), Ala, Phe, Lys(Ac), Dab(Ac), Cit, and Orn.

In some embodiments, X6 is Gln. In some embodiments, X6 is Q(Me). In some embodiments, X6 is Q(2Me). In some embodiments, X6 is Q(pyrrolidin). In some embodiments, X6 is Ala. In some embodiments, X6 is Phe. In some embodiments, X6 is Lys(Ac). In some embodiments, X6 is Dab(Ac). In some embodiments, X6 is Cit. In some embodiments, X6 is Orn.

Preferably, X6 is Gln.

### X7

X7 is selected from Glu, D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys, N-Me-homo-Cys.

In some embodiments, X7 is Dab or Cys. The amino acid residue at X7 forms a lactam bridge or a dithioether bridge with the amino acid residue at X4.

In some embodiments, X7 is Dab. When X7 is Dab, the Dab at X7 forms a lactam bridge with the amino acid residue at X4.

In some embodiments, X7 is Cys. When X7 is Cys, the Cys at X7 forms a dithioether bridge with the amino acid residue at X4.

Preferably, X7 is Dab.

### X8

X8 is selected from the group consisting of an optionally substituted tryptophan residue, an optionally substituted azatryptophan residue, an optionally substituted beta-homotryptophan residue, an optionally substituted tyrosine residue, an optionally substituted phenylalanine residue, an optionally substituted homophenylalanine residue, and an alanine residue substituted by an optionally substituted carbocylic group or an aromatic group selected from the group consisting of phenyl, pyridyl, naphthyl and quinolinyl, each optionally substituted.

In some embodiments, X8 is selected from the group consisting of Y(2-aminoethoxy), homo-Phe, 7F(4-morpholine), 3-quinolinylalanine, Y(2-aminoethoxy)(N(Me)2, Y(nPentylamine)(N⁺(Me)3), Y(2-trimethyl-PEG2), Y(2-aminoethoxy)(N(Me)2), 7-AzaTrp, beta-homo-Trp, and 7-F-Trp.

In some embodiments, X8 is selected from the group consisting of Y(2-aminoethoxy), homo-Phe, 7-AzaTrp, beta-homo-Trp, and 7-F-Trp.

In some embodiments, X8 is Y(2-aminoethoxy). In some embodiments, X8 is homo-Phe. In some embodiments, X8 is 7-AzaTrp. In some embodiments, X8 is beta-homo-Trp. In some embodiments, X8 is 7-F-Trp.

Preferably, X8 is Y(2-aminoethoxy).

### X9

X9 is selected from the group consisting of an optionally substituted tryptophan residue, an optionally substituted azatryptophan residue, an optionally substituted beta-homotryptophan residue, an optionally substituted alanine residue, an optionally substituted phenylalanine and an optionally substituted tyrosine residue.

In some embodiments, X9 is selected from the group consisting of 2-Nal, 7-AzaTrp, beta-homo-Trp, 3-quinolinylalanine and cyclopropyl-Ala.

In some embodiments, X9 is selected from the group consisting of 2-Nal, 7-AzaTrp, beta-homo-Trp, and Cyclopropyl-Ala.

In some embodiments, X9 is 2-Nal. In some embodiments, X9 is 7-AzaTrp. In some embodiments, X9 is beta-homo-Trp. In some embodiments, X9 is Cyclopropyl-Ala.

Preferably, X9 is 2-Nal.

### X10

X10 is selected from the group consisting of Val, D-Val, beta-Val, D-beta-Val, homo-Val, D-homo-Val, beta-homo-Val, N-Me-Val, N-Me-homo-Val, 2-Me-Val,
Gly, beta-Gly, homo-Gly, beta-homo-Gly, N-Me-Gly, N-Me-homo-Gly,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, 2-Me-Lys,
Aib, D-Aib, beta-Aib, D-beta-Aib, homo-Aib, D-homo-Aib, beta-homo-Aib, N-Me-Aib, N-Me-homo-Aib,
Ala, D-Ala, beta-Ala, D-beta-Ala, homo-Ala, D-homo-Ala, beta-homo-Ala, N-Me-Ala, N-Me-homo-Ala,
Leu, D-Leu, beta-Leu, D-beta-Leu, homo-Leu, D-homo-Leu, beta-homo-Leu, N-Me-Leu, N-Me-homo-Leu, 2-Me-Leu,
Ile, D-Ile, beta-Ile, D-beta-Ile, homo-Ile, D-homo-Ile, beta-homo-Ile, N-Me-Ile, N-Me-homo-Ile,
and a carbocyclic or heterocyclic ring having an amino substituent and a carbonyl substituent.

In some embodiments, X10 is selected from the group consisting of 2-Me-Leu or Aib.

In some embodiments, X10 is 2-Me-Leu. In some embodiments, X10 is Aib.

Preferably, X10 is 2-Me-Leu.

The inventors have observed that having an amino acid addressing SIF stability (that is, gastrointestinal stability) at position 10 (X10) is necessary. The amino acids 2-Me-Leu and Aib, particularly 2-Me-Leu, address SIF stability. If, for example, the amino acid residue is Gly in position 10 (X10), the inventors observed that the SIF stability of the compound is significantly decreased.

### X11

X11 is selected from the group consisting of
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys, D-lys, iso-Lys, D-iso-Lys, beta-Lys, D-beta-Lys, homo-Lys, D-homo-Lys, beta-homo-Lys, N-Me-Lys, N-Me-homo-Lys, Lys(Me),
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl.

In some embodiments, X11 is Glu.

The Glu residue at X11 forms a lactam bridge with the amino acid residue at either X1 or X2 when X1 is absent. In some embodiments, the Glu residue at X11 forms a lactam bridge with the amino acid residue at X1. In some embodiments, the Glu residue at X11 forms a lactam bridge with the amino acid residue at X2.

When X1 is absent, the Glu residue at X11 forms a lactam bridge with the amino acid residue at X2.

### X12

X12 is selected from the group consisting of an optionally substituted Phe residue, an optionally substituted Tyr residue, an optionally substituted His residue, an alanine residue substituted by a carbocyclic group or an aromatic or heteroaromatic group selected from the group consisting of phenyl, pyridyl, naphthyl and quinolinyl, each optionally substituted, Dab, D-Dab, iso-Dab, D-iso-Dab, beta-Dab, D-beta-Dab, homo-Dab, D-homo-Dab, beta-homo-Dab, N-Me-Dab, N-Me-homo-Dab, Gly, beta-Gly, homo-Gly, beta-homo-Gly, N-Me-Gly, N-Me-homo-Gly, Pro, 5-aminopentanoyl, 4-aminopiperidin-4-carbonyl, (R,S)-Imidazolidin-2-carbonyl, 3-aminopropanoyl, Gly-CF3, D-Gly-CF3, Nle, Gln, D-Gln, iso-Gln, D-iso-Gin, beta-Gln, D-beta-Gin, homo-Gln, D-homo-Gln, beta-homo-Gin, N-Me-Gln, N-Me-homo-Gln, THP, Ser, D-Ser, beta-Ser, D-beta-Ser, homo-Ser, D-homo-Ser, beta-homo-Ser, N-Me-Ser, 2-Me-Ser, N-Me-homo-Ser, Ser(OMe), 3-aminotetrahydrofuran-3-carbonyl, THP, Arg, D-Arg, beta-Arg, D-beta-Arg, homo-Arg, D-homo-Arg, beta-homo-Arg, N-Me-Arg, N-Me-homo-Arg, Thr, D-Thr, beta-Thr, D-beta-Thr, homo-Thr, D-homo-Thr, beta-homo-Thr, N-Me-Thr, N-Me-homo-Thr Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Asn, D-Asn, beta-Asn, D-beta-Asn, homo-Asn, D-homo-Asn, beta-homo-Asn, N-Me-Asn, N-Me-homo-Asn, GABA, 2-(trimethyl-2-aminoethoxy)ethoxy]propyl and Lys wherein the side chain —NH₂ of the Lys is substituted with —C(═O)(CH₂)ₙR^{K} wherein n is 0-2 and R^{K} is imidazolyl, pyrimidyl, or pyridyl optionally substituted with F.

In some embodiments, X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser.

In some embodiments, X12 is selected from the group consisting of Dab, D-Arg, and Ser. In some embodiments, X12 is Dab or Ser. In some embodiments, X12 is Dab or D-Arg.

In some embodiments, X12 is Dab. In some embodiments, X12 is iso-Dab. In some embodiments, X12 is D-Arg. In some embodiments, X12 is Gly-CF3. In some embodiments, X12 is D-Gly-CF3. In some embodiments, X12 is Ne. In some embodiments, X12 is Gln. In some embodiments, X12 is His. In some embodiments, X12 is THP. In some embodiments, X12 is Ser. In some embodiments, X12 is D-Ser. In some embodiments, X12 is 2-Me-Ser. In some embodiments, X12 is Ser(OMe). In some embodiments, X12 is homo-Ser.

Preferably, X12 is Dab.

### X13

X13 is selected from the group consisting of an optionally substituted His residue, an optionally substituted Phe residue, an alanine residue substituted by a carbocyclic group or an aromatic or heteroaromatic group selected from the group consisting of phenyl, pyridyl, naphthyl and quinolinyl, each optionally substituted, Asn, D-Asn, beta-Asn, D-beta-Asn, homo-Asn, D-homo-Asn, beta-homo-Asn, N-Me-Asn, N-Me-homo-Asn, Gly, beta-Gly, homo-Gly, beta-homo-Gly, N-Me-Gly, N-Me-homo-Gly, and Dab, Orn or Lys wherein the side chain —NH₂ is substituted with —C(═O)(CH₂)ₙR^{K} wherein n is 0-2 and R^{K} is imidazolyl, pyrimidyl, or pyridyl optionally substituted with F or is absent.

In some embodiments, X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent.

In some embodiments, X13 is 3-(3-Pyridyl)-Ala or is absent. When X13 is absent, X14 is also absent.

In some embodiments, X13 is 3-(3-Pyridyl)-Ala. In some embodiments, X13 is D-3-(3-Pyridyl)-Ala. In some embodiments, X13 is 2-Me-3-(3-Pyridyl)-Ala. In some embodiments, X13 is N-Me-3-(3-Pyridyl)-Ala. In some embodiments, X13 is 3-(3,5-Pyrimidyl)-Ala. In some embodiments, X13 is His. In some embodiments, X13 is D-His. In some embodiments, X13 is His(Me). In some embodiments, X13 is absent.

Preferably, X13 is 3-(3-Pyridyl)-Ala or is absent. Even more preferably, X13 is 3-(3-Pyridyl)-Ala.

### X14

X14 is absent or is selected from the group consisting of Sar, an optionally substituted His residue, Leu, D-Leu, beta-Leu, D-beta-Leu, homo-Leu, D-homo-Leu, beta-homo-Leu, N-Me-Leu, N-Me-homo-Leu, 2-Me-Leu, and an alanine residue substituted by a carbocyclic group or an aromatic or heteroaromatic group selected from the group consisting of phenyl, pyridyl, naphthyl and quinolinyl, each optionally substituted.

In some embodiments, X14 is absent or is selected from the group consisting of Sar, D-His, beta-homo-Leu, and 3-(3-Pyridyl)-Ala.

In some embodiments, X14 is absent. In such embodiments where X14 is absent, X13 may preferably be 3-(3-Pyridyl)-Ala.

In some embodiments, X14 is Sar. In some embodiments, X14 is D-His. In some embodiments, X14 is beta-homo-Leu. In some embodiments, X14 is 3-(3-Pyridyl)-Ala. When X14 is present, that is when X14 is selected from the group consisting of Sar, D-His, beta-homo-Leu, and 3-(3-Pyridyl)-Ala; X13 may be 3-(3-Pyridyl)-Ala or His.

Preferably, X14 is absent.

### Lactam Bridge

A lactam bridge is formed of one amino acid residue comprising an amine group and another amino acid residue comprising a carboxylic acid group. The amine and/or carboxylic acid group of the amino acid residue may be on the side chain of the amino acid residue, such as Dab and Glu. Alternatively, the amine and/or carboxylic acid group of the amino acid residue may be the N- or C-terminus of the peptide chain, such as the amine or carboxylic acid of the peptide backbone of any amino acid, such as 4-aminomethyl-phenylacetyl, 3-(3-Pyridyl)-Ala, and 3-aminopropanoyl.

One of the amino acid residues comprise an amine group and the other amino acid residue comprises a carboxylic acid group, wherein a lactam (cyclic amide) is formed between the amine and carboxylic acid groups.

For simplicity, the amino acid residues who together form a lactam bridge will be discussed by reference to the residues nominally present before lactam formation.

Suitable amino acid residues who together form a lactam bridge may be selected from:
Amino acid residues comprising an amine group such as: 4-aminomethyl-phenylacetyl, 3-(3-Pyridyl)-Ala, 3-aminopropanoyl, and Dab.
Amino acid residues comprising a carboxylic acid group such as: Glu.

The inventors have found that replacing a dithioether bridge with a lactam bridge can lead to increased potency of the IL-23R peptide inhibitor, as previously described in Example 2 of WO2023/099669.

### First Lactam Bridge—X1/X2 and X11

A first lactam bridge is formed between the residue at X11 with the amino acid residue at X1 or at X2 when X1 is absent. In some embodiments, the first lactam bridge is formed between the residue at X11 with the amino acid residue at X1. In some embodiments, the first lactam bridge is formed between the residue at X11 with the amino acid residue at X2. When X1 is absent, the first lactam bridge is formed between the residue at X11 with the amino acid residue at X2. Preferably, the first lactam bridge is formed between the residue at X11 with the amino acid residue at X2.

In some embodiments, A first lactam bridge is formed between the Glu at X11 with the amino acid residue at X1 or at X2 when X1 is absent. In some embodiments, the first lactam bridge is formed between the Glu at X11 with the amino acid residue at X1. In some embodiments, the first lactam bridge is formed between the Glu at X11 with the amino acid residue at X2. When X1 is absent, the first lactam bridge is formed between the Glu at X11 with the amino acid residue at X2. Preferably, the first lactam bridge is formed between the Glu at X11 with the amino acid residue at X2.

In embodiments where the first lactam bridge is formed between the Glu at X11 with the amino acid residue at X1, X1 is 3-(3-Pyridyl)-Ala or 3-aminopropanoyl. That is, the following combinations of X1 and X11 form a lactam bridge:
X1 is 3-(3-Pyridyl)-Ala and X11 is Glu; and
X1 is 3-aminopropanoyl and X11 is Glu.

In embodiments where the first lactam bridge is formed between the Glu at X11 with the amino acid residue at X2, X2 is 4-aminomethyl-phenylacetyl, 3-(3-Pyridyl)-Ala, and 3-aminopropanoyl. That is, the following combinations of X2 and X11 form a lactam bridge:
X2 is 4-aminomethyl-phenylacetyl and X11 is Glu;
X2 is 3-(3-Pyridyl)-Ala and X11 is Glu; and
X2 is 3-aminopropanoyl and X11 is Glu.

In such combinations, X1 is absent.

### Second Lactam Bridge—X4 and X7

A second lactam bridge may be formed between the amino acid residues at X4 and X7. In some such embodiments, X4 is Glu and X7 is Dab.

Preferably, the bridge between the amino acid residues at X4 and X7 is a lactam bridge.

### Dithioether Bridge

A dithioether bridge is formed of two amino acid residues comprising sulfur moieties, such as —SH. Preferably, the sulfur moiety of the amino acid residue is on the side chain of the amino acid residue, such as Cys.

For simplicity, the amino acid residues who together form a dithioether bridge will be discussed by reference to the residues nominally present before dithioether formation.

Suitable amino acid residues who together form a dithioether bridge may be Cys.

The inventors have found that a linker is required between the thiol side chains of linked [4,7] cysteine residues in the compounds to retain IL-23R inhibitory activity. Replacing the dithioether bridges with disulfide bridges resulted in loss of inhibitory activity (see Example 2 of WO2023/099669).

### Dithioether Formula

In some embodiments, the dithioether bridge is of the formula —S-L-Y-L-S—, wherein:
each S is a sulfur atom and is part of one of the amino acid residues forming the dithioether bridge;
each L is independently C₁₋₄ alkylene; and
Y is either absent or C(═O).

### L

Each L is independently C₁₋₄ alkylene.

In some embodiments, L is C₁₋₂ alkylene. In some embodiments, L is C₁ alkylene (methylene or —CH₂—). In some embodiments, L is C₂ alkylene (ethylene or —CH₂CH₂—).

### Y

Y is either absent or C(═O).

In some embodiments, Y is absent. In some embodiments, Y is C(═O). Preferably, Y is C(═O).

### Preferred Dithioether Bridges

In some embodiments, the dithioether bridge is of the formula —SCH₂C(═O)CH₂S—, wherein each S is a sulfur atom and is part of one of the amino acid residues forming the dithioether bridge.

### Dithioether Bridge—X4 and X7

A dithioether bridge may be formed between the amino acid residues at X4 and X7. In such embodiments, X4 is Cys and X7 is Cys.

In embodiments where a dithioether bridge is formed between the amino acid residues at X4 and X7, preferably the dithioether bridge between X4 and X7 is of the formula —S-L-Y-L-S—, wherein:
each S is a sulfur atom and is part of the amino acid residue at X4 and X7;
each L is independently C₁₋₄ alkylene; and
Y is either absent or C(═O).

In embodiments where a dithioether bridge is formed between the amino acid residues at X4 and X7, even more preferably the bridge is of the formula —SCH₂C(═O)CH₂S—, wherein each S is a sulfur atom and is part of the amino acid residue at X4 and X7. That is, each L is —CH₂— and Y is C(═O).

### Bridge Length

The length of the bridge is counted as the number of atoms in a linear chain from the first atom attached to the atom (carbon) adjacent to the carboxylic acid moiety of the amino acid of the first residue (X1/X2 for the bridge between X1/X2 and X11; or X4 for the bridge between X4 and X7), i.e. attached to the alpha carbon of the relevant residue for most amino acids, up to the first atom attached to the atom (carbon) adjacent to the carboxylic acid moiety of the amino acid of the second residue (X11 for the bridge between X1/X2 and X11; or X7 for the bridge between X4 and X7).

The contribution to the length of the bridge for amino acid residues and the type of bridge is described below.

In some embodiments, the length of the bridge between X1/X2 and X11 is at least 4 atoms long. In some embodiments, the length of the bridge between X1/X2 and X11 is no longer than 10 atoms long. In some embodiments, the length of the bridge between X1/X2 and X11 is 4 to 10 atoms long, such as 4, 5, 6, 7, 8, 9, or 10 atoms long. In some embodiments, the length of the bridge between X1/X2 and X11 is 4 to 9 atoms long, such as 4, 5, 6, 7, 8, or 9 atoms long. In some embodiments, the length of the bridge between X1/X2 and X11 is either 4, 5, or 9 atoms long. In some embodiments, the length of the bridge between X1/X2 and X11 is 4 atoms long. In some embodiments, the length of the bridge between X1/X2 and X11 is either 5 atoms long. In some embodiments, the length of the bridge between X1/X2 and X11 is 9 atoms long.

In some embodiments, the length of the bridge between X4 and X7 is at least 4 atoms long. In some embodiments, the length of the bridge between X4 and X7 is no longer than 10 atoms long. In some embodiments, the length of the bridge between X4 and X7 is 4 to 10 atoms long, such as 4, 5, 6, 7, 8, 9, or 10 atoms long. In some embodiments, the length of the bridge between X4 and X7 is 4 to 9 atoms long, such as 4, 5, 6, 7, 8, or 9 atoms long. In some embodiments, the length of the bridge between X4 and X7 is 6 or 7 atoms long. In some embodiments, the length of the bridge between X4 and X7 is 6 atoms long. In some embodiments, the length of the bridge between X4 and X7 is 7 atoms long.

### Lactam Bridge

The contribution of the side chain to the length of the lactam bridge is counted as the number of atoms in a linear chain from the first atom of the side chain (which is bonded to an atom of the peptide backbone, i.e. to the alpha carbon of the relevant residue for most amino acids) up to and including the atom which participates in the amide bond of the lactam bridge (i.e. the carbon atom of the carboxylic acid functional group or the nitrogen atom of the amine group).

Thus common acid- and amine-containing side chains are considered to have the following side chain lengths It will be understood that the following can be used to decipher the bridge length for any amino acid residue disclosed herein.

### Amine-Containing Side Chains:

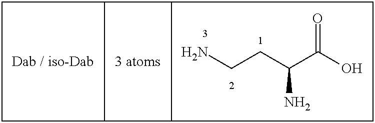

### Carboxylic Acid-Containing Side Chains:

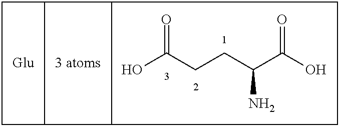

Similarly, the contribution of the length of the N- or C-terminus amino acid residue to the length of the lactam bridge is counted as the number of atoms in a linear chain from the first atom attached to the atom (carbon) adjacent to the carboxylic acid moiety of the amino acid residue (i.e. the first atom attached to the alpha carbon of the relevant residue for most amino acids), up to and including the atoms which participate in the amide bond of the lactam bridge (i.e. the carbon atom of the carboxylic acid functional group or the nitrogen atom of the amine group).

Thus the following amino acid residues are considered to have the following lengths:

### N-Terminus:

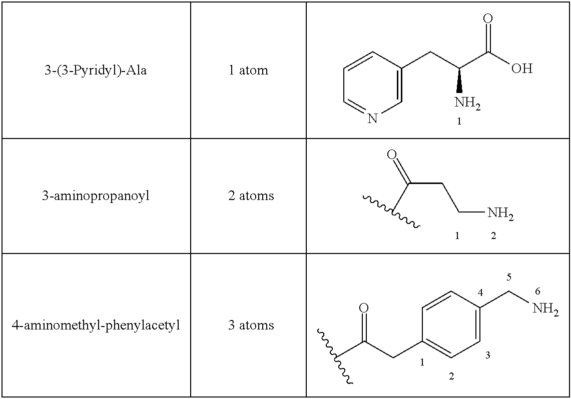

The location of the amide bond in the lactam bridge may affect the potency of the compound. The inventors observed that the compound was more active when the amide bond was closer to position 11 (X11) and less active when closer to position 2 (X2) (see Example 2 and Table 2-3b of WO2023/099669).

Suitable pairings of residues at positions X1/X2 and X11 in which the location of the amide bond in the lactam bridge is closer to position X11 than position X1/X2 once formed include:
X2 is 4-aminomethyl-phenylacetyl and X11 is Glu.

Alternatively, suitable pairings of residues at positions X1/X2 and X11 in which location of the amide bond in the lactam bridge is closer to position X1/X2 than position X11 include:
X1 is 3-(3-Pyridyl)-Ala and X11 is Glu;
X1 is 3-aminopropanoyl and X11 is Glu;
X2 is 3-(3-Pyridyl)-Ala and X11 is Glu; and
X2 is 3-aminopropanoyl and X11 is Glu.

The location of the amide bond in the lactam bridge between position 4 (X4) and position 7 (X7) may be equal distance between X4 and X7, such as when X4 is Glu and X7 is Dab.

Desirably, the length of the lactam bridge after formation of the amide bond (not including any atoms in the peptide backbone) is 4, 5, 6, 7, 8, 9, or 10 atoms; such as 4, 5, 6, or 9 atoms. For the lactam bridge between the amino acid residues at positions X1/X2 and X11, the length of the lactam bridge after formation of the amide bond may be 4, 5, or 9 atoms long. For the lactam bridge between the amino acid residues at positions X4 and X7, the length of the lactam bridge after formation of the amide bond may be 6 atoms long.

In some embodiments, the length of the lactam bridge after formation of the amide bond (not including any atoms in the peptide backbone) is 4 atoms. A suitable pairing of residues at positions X1/X2 and X11 in which the lactam bridge has a length of 4 atoms include: 3-(3-Pyridyl)-Ala and Glu.

In some embodiments, the length of the lactam bridge after formation of the amide bond (not including any atoms in the peptide backbone) is 5 atoms. A suitable pairing of residues at positions X1/X2 and X11 in which the lactam bridge has a length of 5 atoms include: 3-aminopropanoyl and Glu.

In some embodiments, the length of the lactam bridge after formation of the amide bond (not including any atoms in the peptide backbone) is 6 atoms. A suitable pairing of residues at positions X4 and X7 in which the lactam bridge has a length of 6 atoms include: Glu and Dab.

In some embodiments, the length of the lactam bridge after formation of the amide bond (not including any atoms in the peptide backbone) is 7 atoms.

Preferably, the length of the lactam bridge after formation of the amide bond (not including any atoms in the peptide backbone) is 8 atoms.

In some embodiments, the length of the lactam bridge after formation of the amide bond (not including any atoms in the peptide backbone) is 9 atoms. A suitable pairing of residues at positions X1/X2 and X11 in which the lactam bridge has a length of 9 atoms include: 4-aminomethyl-phenylacetyl and Glu.

In some embodiments, the length of the lactam bridge after formation of the amide bond (not including any atoms in the peptide backbone) is 10 atoms.

### Dithioether Bridge

The contribution of the side chain to the length of the dithioether bridge is counted as the number of atoms in a linear chain from the first atom of the side chain (which is bonded to an atom of the peptide backbone, i.e. to the alpha carbon of the relevant residue for most amino acids) up to and including the atom which participates in the dithioether bond of the bridge (i.e. the sulfur atom).

Thus common sulfur-containing side chains are considered to have the following side chain lengths It will be understood that the following can be used to decipher the bridge length for any amino acid residue disclosed herein.

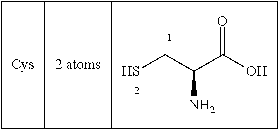

Preferably, X4 is Cys and X7 is Cys.

The length of the side chains of X4 and X7 are then added to the contribution of the linker between the sulfur moieties of the amino acid residues at positions X4 and X7 to determine the length of the bridge. For embodiments where the dithioether bridge is of the formula —S-L-Y-L-S—, the contribution of the linker is counted as the number of atoms in -L-Y-L-, as the sulfur atoms are already counted in the side chain length.

Desirably, the length of the dithioether bridge after the formation of the dithioether bonds (not including any atoms in the peptide backbone) is 5, 6, 7, 8, 9, or 10 atoms, such as 6, 7, 8, or 9 atoms; such as 7, 8, or 9 atoms, such as 7 or 8 atoms.

In some embodiments, the length of the dithioether bridge is 5 atoms long. In some embodiments, the length of the dithioether bridge is 6 atoms long.

In some embodiments, the length of the dithioether bridge is 7 atoms long, such as for the bridge —SCH₂C(═O)CH₂S—.

In some embodiments, the length of the dithioether bridge is 8 atoms long. In some embodiments, the length of the dithioether bridge is 9 atoms long. In some embodiments, the length of the dithioether bridge is 10 atoms long.

Preferably, the dithioether bridge is 7 atoms long.

### Synthesis of the Compounds

The invention further provides a method of synthesis of a compound of the invention. The compounds (which may also be referred to as peptides) may suitably be manufactured by standard synthetic methods. Thus, the peptides may be synthesized by, e.g., methods comprising synthesizing the peptide by standard solid-phase or liquid-phase methodology, either stepwise or by fragment assembly, and optionally isolating and purifying the final peptide product. In this context, reference may be made to WO 98/11125 or, inter alia, Fields, G. B. et al., “Principles and Practice of Solid-Phase Peptide Synthesis”; in: Synthetic Peptides, Gregory A. Grant (ed.), Oxford University Press (2^{nd} edition, 2002) and the synthesis examples herein. The method typically further comprises the step of forming an amide bond between the amino acid residues at position 1 (X1) or at position 2 (X2) when the amino acid residue at position 1 is absent and at position 11 (X11), and optionally further comprising the step of forming an amide bond or forming two thioether bonds with a linker between the amino acid residues at positions 4 (X4) and 7 (X7), e.g. as described below. In the case of solid phase synthesis, cyclisation may be performed in situ on the solid phase (e.g. resin), i.e. before removal of the peptide from the solid phase.

The synthesis of some example compounds of the invention are provided in Example 1. Generally, the method for the synthesis of said compound comprises synthesising the compound by solid-phase or liquid-phase peptide synthesis methodology, optionally isolating and/or purifying the final product, and optionally further comprising the step of forming an amide bond between the amino acid residues at position 1 or at position 2 when the amino acid residue at position 1 is absent and at position 11, and optionally further comprising the step of forming an amide bond or forming two thioether bonds with a linker between the amino acid residues at positions 4 and 7.

The order of the steps in the synthesis of the compounds are not necessarily in the order mentioned above.

For example, the order of the formation of the bridges (lactam bridge/amide bond; dithioether bridge/two thioether bonds) may be in any order. In some embodiments, the bridge between the amino acid residues at position 1 or at position 2 when the amino acid residue at position 1 is absent and at position 11 is formed first, then the bridge between the amino acid residues at positions 4 and 7 is formed second. In other embodiments, the bridge between the amino acid residues at positions 4 and 7 is formed first, then the bridge between the amino acid residues at position 1 or at position 2 when the amino acid residue at position 1 is absent and at position 11 is formed second.

### Efficacy of the Compounds

The compounds of the invention are interleukin-23 receptor (IL-23R) inhibitors, i.e. they are capable of binding to, and blocking signalling by, one or more receptors or receptor complexes regarded as physiological receptors for interleukin-23 (IL-23).

Comparative activity may be measured by any suitable means, such as via determination of IC₅₀ values as described below.

Compounds of the present invention may exhibit a number of advantageous properties in relation to other peptide IL-23R inhibitors thereof, such as analogues described in WO 2016/011208, WO 2018/022937, WO 2018/136646, WO 2020/014646, WO 2021/146441, WO 2021/146458, WO 2023/288017, WO 2023/288019, WO 2023/288028, Kong et al., 2020, WO 2023/099669 and WO 2024/015958. As compared to any of these analogues, compounds of the invention may, for example, exhibit improved effects, e.g., in the form of improved in vitro potency at IL-23R.

Additionally or alternatively, compounds of the invention may exhibit improved gastrointestinal (GI) stability as compared to any of the peptide inhibitors of IL-23R described in the art.

The skilled person will be aware of suitable assay formats, and examples are provided below. For example, the assays may make use of employing measurements on the human IL-23R (see the examples below). Where sequences of precursor proteins are referred to, it should be understood that assays may make use of the mature protein, lacking the signal sequence.

K_{d} values may be used as a numerical measure of the binding affinity at a given receptor. A K_{d} value, also termed the equilibrium dissociation constant, is a measure of how tightly a compound binds to a receptor in a particular assay. A small K_{d} indicates the compound binds tighter with higher affinity to the receptor as compared to a compound with a higher K_{d} value. Thus, for example, a compound having a K_{d} [IL-23R] value lower than the K_{d}[IL-23R] value of another compound inhibitor of IL-23R in a particular assay may be considered to have a stronger binding affinity (or binds more tightly) to IL-23R than that of the other compound inhibitor of IL-23R.

In absence of an experimental method to directly determine the K_{d} of a compound for a receptor, the binding affinities for compounds may be estimated by means of determining the IC₅₀ of a compound. The IC₅₀ is determined by the compound's ability to compete with a labelled compound for the receptor. In such a competition assay, the concentration for which half of the labelled compound is displaced from the receptor by the unlabelled compound is termed the IC₅₀ value. The IC₅₀ value is proportional to the affinity of the compound for the receptor (that is, its K_{d} value), and is assay system dependent as it depends on factors such as the concentration of the labelled compound used, the affinity of the labelled compound for the receptor, and assay incubation times.

In a binding assay format an IC₅₀ value may be used as a numerical format to measure how tightly a compound binds to a receptor in a particular assay. A small IC₅₀ indicates the compound binds tighter with higher affinity to the receptor as compared to a compound with a higher IC₅₀ value. Thus, for example, a compound having a IC₅₀ [IL-23R] value lower than the IC₅₀ [IL-23R] value of another compound inhibitor of IL-23R in a particular assay may be considered to have a stronger binding affinity (or binds more tightly) to IL-23R than that of the other compound inhibitor of IL-23R.

In some embodiments of compounds of the present invention, the IC₅₀ towards IL-23R is below 1000 nM (e.g. 0.0001 to 1000 nM).

In some embodiments of compounds of the present invention, the IC₅₀ towards IL-23R is below 500 nM (e.g. 0.0001 to 500 nM).

In some embodiments of compounds of the present invention, the IC₅₀ towards IL-23R is below 100 nM (e.g. 0.0001 to 100 nM).

In some embodiments of compounds of the present invention, the IC₅₀ towards IL-23R is below 50 nM (e.g. 0.0001 to 50 nM).

In some embodiments of compounds of the present invention, the IC₅₀ towards IL-23R is below 30 nM (e.g. 0.0001 to 30 nM).

In some embodiments of compounds of the present invention, the IC₅₀ towards IL-23R is below 20 nM (e.g. 0.0001 to 20 nM).

In some embodiments of compounds of the present invention, the IC₅₀ towards IL-23R is below 10 nM (e.g. 0.0001 to 10 nM).

In some embodiments of compounds of the present invention, the IC₅₀ towards IL-23R is below 5 nM (e.g. 0.0001 to 5 nM).

In some embodiments of compounds of the present invention, the IC₅₀ towards IL-23R is below 1 nM (e.g. 0.0001 to 1 nM).

In some embodiments of compounds of the present invention, the IC₅₀ towards IL-23R is below 0.5 nM (e.g. 0.0001 to 0.5 nM).

In a functional assay format, measuring the ability of compounds to inhibit IL-23 mediated signalling in a cell-based assay, IC₅₀ values may be used as a numerical measure of inhibitor potency. An IC₅₀ value is a measure of the concentration of a compound required to achieve half of that compound's maximal activity in a particular assay. Thus, for example, a compound having an IC₅₀ [IL-23R] value lower than that of another compound inhibitor of IL-23R in a particular assay may be considered to have a stronger inhibitory potency, presumably by better blocking of the IL-23 mediated signalling, than that of the other peptide inhibitor of IL-23R.

In some embodiments of compounds of the present invention, the IC₅₀ towards IL-23 mediated signalling is below 1000 nM (e.g. 0.0001 to 1000 nM).

In some embodiments of compounds of the present invention, the IC₅₀ towards IL-23 mediated signalling is below 500 nM (e.g. 0.0001 to 500 nM).

In some embodiments of compounds of the present invention, the IC₅₀ towards IL-23 mediated signalling is below 100 nM (e.g. 0.0001 to 100 nM).

In some embodiments of compounds of the present invention, the IC₅₀ towards IL-23 mediated signalling is below 50 nM (e.g. 0.0001 to 50 nM).

In some embodiments of compounds of the present invention, the IC₅₀ towards IL-23 mediated signalling is below 30 nM (e.g. 0.0001 to 30 nM).

In some embodiments of compounds of the present invention, the IC₅₀ towards IL-23 mediated signalling is below 20 nM (e.g. 0.0001 to 20 nM).

In some embodiments of compounds of the present invention, the IC₅₀ towards IL-23 mediated signalling is below 10 nM (e.g. 0.0001 to 10 nM).

In some embodiments of compounds of the present invention, the IC₅₀ towards IL-23 mediated signalling is below 5 nM (e.g. 0.0001 to 5 nM).

In some embodiments of compounds of the present invention, the IC₅₀ towards IL-23 mediated signalling is below 1 nM (e.g. 0.0001 to 1 nM).

In some embodiments of compounds of the present invention, the IC₅₀ towards IL-23 mediated signalling is below 0.5 nM (e.g. 0.0001 to 0.5 nM).

Such assays may be performed under the conditions described in Examples 3-1 and 3-2 below.

Additionally or alternatively, compounds of the invention may show gastrointestinal (GI) stability, i.e. resistance to degradation in the gastrointestinal tract. This can be measured using a simulated intestinal fluid (SIF) assay and/or a simulated gastric fluid (SGF) assay. For example, the compounds of the invention may retain at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the remaining compound or peptide after incubation for 1 hour, or for 4 hours, e.g. under the conditions described in Examples 3-3 and 3-4. Preferably, the compounds retain at least 70% (or more) of the compound after incubation for 1 hour, or 4 hours, under the SIF and/or SGF assay.

### Pharmaceutical Compositions

The invention also extends to compositions, such as pharmaceutical compositions, comprising the compounds of the invention. As with all aspects of the invention, it is to be understood that reference to a compound of the invention encompasses reference to pharmaceutically acceptable salts and solvates.

The compounds of the present invention may be formulated as pharmaceutical compositions which are suited for administration with or without storage, and which typically comprise a therapeutically effective amount of at least one peptide of the invention, together with a pharmaceutically acceptable carrier, excipient or vehicle.

The pharmaceutical composition may be for any mode of administration common or standard in the art, e.g. oral, intravenous, intramuscular, subcutaneous, sublingual, intranasal, intradermal, suppository routes or implanting. In a preferred embodiment of the invention as described herein the pharmaceutical composition is a composition for oral administration.

The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17^{th} edition, Alfonso R. Gennaro (Ed.), Mark Publishing Company, Easton, PA, USA, 1985.

### Therapeutic Uses

The compounds of the invention, and pharmaceutical compositions comprising said compounds, are useful in a method of prevention or treatment of various conditions.

The method of prevention or treatment comprises administering to the subject a therapeutically effective amount of the compound of the invention, or the pharmaceutical composition comprising said compound.

In some embodiments, the conditions may be selected from Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, and graft versus host disease. In a preferred embodiment, the conditions may be selected from inflammatory bowel disease (IBD) such as Crohn's Disease or ulcerative colitis, psoriatic arthritis, and psoriasis.

In some embodiments, the conditions may be selected from inflammatory bowel disease (IBD) such as Crohn's Disease or ulcerative colitis, and psoriasis.

In some embodiments, the conditions may be selected from inflammatory bowel disease (IBD) and psoriasis.

The subject or patient may be an animal subject or patient. The subject or patient may be a human subject or patient. Preferably, the subject is a human subject or patient.

### Dosages

A typical dosage of a compound as employed in the context of the present invention may be in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses. The exact dosage employed will depend, inter alia, on: the nature and severity of the disease or disorder to be treated, on the sex, age, body weight and general condition of the subject to be treated, on possible other, concomitant, disease or disorder that is undergoing or is to undergo treatment, as well as on other factors that will be known to a medical practitioner of skill in the art.

The administration of the compounds described herein may be by any mode of administration common or standard in the art, e.g. oral, intravenous, intramuscular, subcutaneous, sublingual, intranasal, intradermal, suppository routes or implanting. In a preferred embodiment of the invention as described herein administration is by oral administration.

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed as limiting the scope of the present invention in any way.

Abbreviations employed for the amino acids and particular R² groups may be found in Tables A-C in the definitions. Other abbreviations employed in the examples include:

| | |
|---|---|
| ^{t}BuOH | tert-Butanol |
| DODT | 2,2′-(Ethylenedioxy)diethanethiol |
| Pd(PPh₃)₄ | tetrakis(triphenylphosphine)palladium(0)) |
| PhSiH₃ | phenylsilane |
| PyBOP | benzotriazol-1-yloxytripyrrolidinophosphonium |
| | hexafluorophosphate) |
| equiv. | equivalents |
| r.t. | room temperature |
| aq. | Aqueous |
| IL-23R | interleukin-23 receptor |
| hIL-23R | human interleukin-23 receptor |
| GI | gastrointestinal |
| SIF | simulated intestinal fluid |
| SGF | simulated gastric fluid |
| Nluc | NanoBRET luciferase assay |
| SD | standard deviation |
| % Eff | percentage efficacy |
| pSTAT3 | phosphorylated signal transducer and activator of |
| | transcription 3 |
| BRET | Bioluminescence Resonance Energy Transfer |
| TAMRA | 5′-tetramethylrhodamine-5-carboxamide |

The following examples are provided to illustrate certain embodiments of the invention and are not intended to limit the scope of the invention.

### Example 1: Synthesis of Compounds

The following compounds were synthesised in Table 1-1 below.

TABLE 1-1
Synthesised compounds

| Compound | Structure |
|---|---|
| 1 | [3-Aminopropanoyl](4c)*[3-(3-Pyridyl)-Ala]SC(2a)WQC(2a)[Y(2- |
| | aminoethoxy)][2-Nal][2-Me-Leu]E(4c)[{d}R]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CSCC(═O)CSC[C@H](NC(═O)[C@H](CO)NC(═O) |
| | [C@H](Cc3cccnc3)NC(═O)CCNC(═O)CC[C@@H](C(═O)N[C@H](CCCNC |
| | (═N)N)C(N)═O)NC1═O)C(═O)N[C@@H](Cc1c[nH]c3ccccc13)C(═O)N[C@@H] |
| | (CCC(N)═O)C(═O)N2 |
| 2 | [3-(3-Pyridyl)-Ala](4c)*[3-Aminopropanoy]SC(2a)WQC(2a)[Y(2- |
| | aminoethoxy)][2-Nal][2-Me-Leu]E(4c)[{d}R]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CSCC(═O)CSC[C@H](NC(═O)[C@H](CO)NC(═O) |
| | CCNC(═O)[C@H](Cc3cccnc3)NC(═O)CC[C@@H](C(═O)N[C@H](CCCNC |
| | (═N)N)C(N)═O)NC1═O)C(═O)N[C@@H](Cc1c[nH]c3ccccc13)C(═O)N[C@@H] |
| | (CCC(N)═O)C(═O)N2 |
| 3 | [4-Aminomethyl-phenylacetyl](1c)*SE(2c)WQ[Dab](2c)[Y(2-aminoethoxy)][2- |
| | Nal][2-Me-Leu]E(1c)[Dab][2-Me-3-(3-Pyridyl)-Ala]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@@](C) |
| | (Cc3cccnc3)C(N)═O)NC1═O)C(═O)N[C@@H](Cc1c[nH]c3ccccc13)C(═O)N[C@@H] |
| | (CCC(N)═O)C(═O)N2 |
| 4 | [4-Aminomethyl-phenylacetyl](1c)*SE(2c)WQ[Dab](2c)[Y(2-aminoethoxy)][2- |
| | Nal][2-Me-Leu]E(1c)[Dab][N—Me-3-(3-Pyridyl)-Ala]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N(C)[C@@H] |
| | (Cc3cccnc3)C(N)═O)NC1═O)C(═O)N[C@@H](Cc1c[nH]c3ccccc13)C(═O) |
| | N[C@@H](CCC(N)═O)C(═O)N2 |
| 5 | [4-Aminomethyl-phenylacetyl](1c)*SE(2c)WQ[Dab](2c)[Y(2-aminoethoxy)][2- |
| | Nal][2-Me-Leu]E(1c)[Dab][{d}3-(3-Pyridyl)-Ala]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@H] |
| | (Cc3cccnc3)C(N)═O)NC1═O)C(═O)N[C@@H](Cc1c[nH]c3ccccc13)C(═O)N[C@@H] |
| | (CCC(N)═O)C(═O)N2 |
| 6 | [4-Aminomethyl-phenylacetyl](1c)*SE(2c)WQ[Dab](2c)[Y(2-aminoethoxy)][2- |
| | Nal][2-Me-Leu]E(1c)[Dab]-[NH-(4-(pyridin-3-yl)butanyl)] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)NCCCCc3cccnc3) |
| | NC1═O)C(═O)N[C@@H](Cc1c[nH]c3ccccc13)C(═O)N[C@@H](CCC(N)═O) |
| | C(═O)N2 |
| 7 | [4-Aminomethyl-phenylacetyl](1c)*SE(2c)WQ[Dab](2c)[Y(2-aminoethoxy)][2- |
| | Nal][2-Me-Leu]E(1c)[Dab]-[NH-(2-(pyridin-3-yl)ethyl)] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)NCCc3cccnc3) |
| | NC1═O)C(═O)N[C@@H](Cc1c[nH]c3ccccc13)C(═O)N[C@@H](CCC(N)═O) |
| | C(═O)N2 |
| 8 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O) |
| | N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 9 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][Sar]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@@H] |
| | (Cc3cccnc3)C(═O)N(C)CC(N)═O)NC1═O)C(═O)N[C@@H](Cc1c[nH] |
| | c3ccccc13)C(═O)N[C@@H](CCC(N)═O)C(═O)N2 |
| 10 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][{d}H]-[NH2] |
| | CC(C)C[C@]1(C)NC(═O)[C@H](Cc2ccc3ccccc3c2)NC(═O)[C@H](Cc2ccc(OCCN) |
| | cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@@H] |
| | (Cc3cccnc3)C(═O)N[C@H](Cc3cnc[nH]3)C(N)═O)NC1═O)C(═O)N[C@@H] |
| | (Cc1c[nH]c3ccccc13)C(═O)N[C@@H](CCC(N)═O)C(═O)N2 |
| 11 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][beta-hLeu]-[NH2] |
| | CC(C)C[C@@H](CC(N)═O)NC(═O)[C@H](Cc1cccnc1)NC(═O)[C@H](CCN) |
| | NC(═O)[C@@H]1CCC(═O)NCc2ccc(cc2)CC(═O)N[C@@H](CO)C(═O)N[C@H] |
| | 2CCC(═O)NCC[C@H](NC(═O)[C@H](CCC(N)═O)NC(═O)[C@H](Cc3c[nH] |
| | c4ccccc34)NC2═O)C(═O)N[C@@H](Cc2ccc(OCCN)cc2)C(═O)N[C@@H] |
| | (Cc2ccc3ccccc3c2)C(═O)N[C@@](C)(CC(C)C)C(═O)N1 |
| 12 | [4-Aminomethyl-phenylacetyl](1c)*SE(2c)WQ[Dab](2c)[Y(2-aminoethoxy)][2- |
| | Nal][2-Me-Leu]E(1c)[Dab]H[3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@@H] |
| | (Cc3cnc[nH]3)C(═O)N[C@@H](Cc3cccnc3)C(N)═O)NC1═O)C(═O)N[C@@H] |
| | (Cc1c[nH]c3ccccc13)C(═O)N[C@@H](CCC(N)═O)C(═O)N2 |
| 13 | [4-Aminomethyl-phenylacetyl](1c)*SE(2c)WQ[Dab](2c)[Y(2-aminoethoxy)][2- |
| | Nal][2-Me-Leu]E(1c)[Dab][{d}H]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@H] |
| | (Cc3cnc[nH]3)C(N)═O)NC1═O)C(═O)N[C@@H](Cc1c[nH]c3ccccc13)C(═O)N |
| | [C@@H](CCC(N)═O)C(═O)N2 |
| 14 | [4-Aminomethyl-phenylacetyl](1c)*SE(2c)WQ[Dab](2c)[Y(2-aminoethoxy)][2- |
| | Nal][2-Me-Leu]E(1c)[Dab][H(Me)]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)N[C@@H](CCN)C(═O)N[C@@H] |
| | (Cc3cn(C)cn3)C(N)═O)NC1═O)C(═O)N[C@@H](Cc1c[nH]c3ccccc13)C(═O)N |
| | [C@@H](CCC(N)═O)C(═O)N2 |
| 15 | [4-Aminomethyl-phenylacetyl](1c)*SE(2c)WQ[Dab](2c)[Y(2-aminoethoxy)][2- |
| | Nal][2-Me-Leu]E(1c)[Iso-Dab][2-Me-3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)NCC[C@H](N)C(═O)N[C@@](C) |
| | (Cc3cccnc3)C(N)═O)NC1═O)C(═O)N[C@@H](Cc1c[nH]c3ccccc13)C(═O)N |
| | [C@@H](CCC(N)═O)C(═O)N2 |
| 16 | [4-Aminomethyl-phenylacetyl](1c)*SE(2c)WQ[Dab](2c)[Y(2-aminoethoxy)][2- |
| | Nal][2-Me-Leu]E(1c)[{d}Gly-CF3][3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)NC[C@@H](N[C@@H](Cc3cccnc3) |
| | C(N)═O)C(F)(F)F)NC1═O)C(═O)N[C@@H](Cc1c[nH]c3ccccc13)C(═O)N |
| | [C@@H](CCC(N)═O)C(═O)N2 |
| 17 | [4-Aminomethyl-phenylacetyl](1c)*SE(2c)WQ[Dab](2c)[Y(2-aminoethoxy)][2- |
| | Nal][2-Me-Leu]E(1c)[Gly-CF3][3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC(═O) |
| | Cc3ccc(cc3)CNC(═O)CC[C@@H](C(═O)NC[C@H](N[C@@H](Cc3cccnc3)C |
| | (N)═O)C(F)(F)F)NC1═O)C(═O)N[C@@H](Cc1c[nH]c3ccccc13)C(═O)N |
| | [C@@H](CCC(N)═O)C(═O)N2 |
| 18 | [4-Aminomethyl-phenylacetyl](1c)*[N—Me-Ser]E(2c)WQ[Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N(C)[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O) |
| | N[C@@](C)(CC(C)C)C(═O)N1 |
| 19 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3,5-Pyrimidyl)-Ala]-[NHMe] |
| | CNC(═O)[C@H](Cc1cncnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | [C@@](C)(CC(C)C)C(═O)N1 |
| 20 | [4-Aminomethyl-phenylacetyl](1c)*AE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](Cc2ccc3ccccc3c2)C(═O)N |
| | [C@@](C)(CC(C)C)C(═O)N1 |
| 21 | [4-Aminomethyl-phenylacetyl](1c)*FE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](Cc2ccccc2)C(═O)N[C@H]2CCC(═O)NCC |
| | [C@H](NC(═O)[C@H](CCC(N)═O)NC(═O)[C@H](Cc3c[nH]c4ccccc34)NC2═O) |
| | C(═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 22 | [4-Aminomethyl-phenylacetyl](1c)*SE(2c)WA[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[c@@H]1CCC(═O) |
| | NCc2ccc(cc2)CC(═O)N[C@@H](CO)C(═O)N[C@H]2CCC(═O)NCC[C@H] |
| | (NC(═O)[C@H](C)NC(═O)[C@H](Cc3c[nH]c4ccccc34)NC2═O)C(═O)N[C@@H] |
| | (Cc2ccc(OCCN)cc2)C(═O)N[C@@H](Cc2ccc3ccccc3c2)C(═O)N[C@@](C) |
| | (CC(C)C)C(═O)N1 |
| 23 | [4-Aminomethyl-phenylacetyl](1c)*SE(2c)WF[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](Cc3ccccc3)NC(═O)[C@H](Cc3c[nH]c4ccccc34)NC2═O)C(═O) |
| | N[C@@H](Cc2ccc(OCCN)cc2)C(═O)N[C@@H](Cc2ccc3ccccc3c2)C(═O)N |
| | [C@@](C)(CC(C)C)C(═O)N1 |
| 24 | [4-Aminomethyl-phenylacetyl](1c)*SE(2c)W[K(Ac)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCCCNC(C)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 25 | [4-Aminomethyl-phenylacetyl](1c)*SE(2c)W[Dab(Ac)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCNC(C)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | [C@@](C)(CC(C)C)C(═O)N1 |
| 26 | [4-Aminomethyl-phenylacetyl](1c)*SE(2c)W[Cit][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCCNC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O) |
| | N[C@@](C)(CC(C)C)C(═O)N1 |
| 27 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCC(═O)N3CCCC3)NC(═O)[C@H](Cc3c[nH]c4ccccc34) |
| | NC2═O)C(═O)N[C@@H](Cc2ccc(OCCN)cc2)C(═O)N[C@@H](Cc2ccc3ccccc3c2) |
| 28 | [4-Aminomethyl-phenylacetyl](1c)*SE(2c)W[Orn][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCCN)NC(═O)[C@H](Cc3c[nH]c4ccccc34)NC2═O)C(═O)N |
| | [C@@H](Cc2ccc(OCCN)cc2)C(═O)N[C@@H](Cc2ccc3ccccc3c2)C(═O)N |
| | [C@@(C)(CC(C)C)C(═O)N1 |
| 29 | [4-Aminomethyl-phenylacetyl](1c)*SE(2c)W[Q(Me)][Dab](2c)[Y(2- |
| | aminoethoxy)][2-Nal][2-Me-Leu]E(1c)[Dab][3-(3-Pyridyl)-Ala]-[NHMe] |
| | CNC(═O)CC[C@@H]1NC(═O)[C@H](Cc2c[nH]c3ccccc23)NC(═O)[C@@H] |
| | 2CCC(═O)NCC[C@H](NC1═O)C(═O)N[C@@H](Cc1ccc(OCCN)cc1)C(═O)N |
| | [C@@H](Cc1ccc3ccccc3c1)C(═O)N[C@@](C)(CC(C)C)C(═O)N[C@H](C(═O) |
| | N[C@@H](CCN)C(═O)N[C@@H](Cc1cccnc1)C(═O)NC)CCC(═O)NCc1ccc |
| | (cc1)CC(═O)N[C@@H](CO)C(═O)N2 |
| 30 | [4-Aminomethyl-phenylacetyl](1c)*SE(2c)W[Q(2Me)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCC(═O)N(C)C)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 31 | [4-Aminomethyl-phenylacetyl](1c)*SE(2c)WQ[Dab](2c)[Y(2-aminoethoxy)][2- |
| | Nal][2-Me-Leu]E(1c)S[3-(3-Pyridyl)-Ala]-[NHMe] |
| | CNC(═O)[C@H](Cc1cccnc1)NC(═O)[C@H](CO)NC(═O)[c@@H]1CCC(═O) |
| | NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O) |
| | N[C@@H](Cc2ccc(OCCN)cc2)C(═O)N[C@@H](Cc2ccc3ccccc3c2)C(═O)N |
| | [C@@[(C)(CC(C)C)C(═O)N1 |
| 32 | [4-Aminomethyl-phenylacetyl](1c)*SE(2c)WQ[Dab](2c)[Y(2-aminoethoxy)][2- |
| | Nal][2-Me-Leu]E(1c)[Nle][3-(3-Pyridyl)-Ala]-[NHMe] |
| | CCCC[C@H](NC(═O)[C@@H]1CCC(═O)NCc2ccc(cc2)CC(═O)N[C@@H] |
| | (CO)C(═O)N[C@H]2CCC(═O)NCC[C@H](NC(═O)[C@H](CCC(N)═O)NC(═O) |
| | [C@H](Cc3c[nH]c4ccccc34)NC2═O)C(═O)N[C@@H](Cc2ccc(OCCN)cc2)C |
| | (═O)N[C@@H](Cc2ccc3ccccc3c2)C(═O)N[C@@](C)(CC(C)C)C(═O)N1)C(═O) |
| | N[C@@H](Cc1cccnc1)C(═O)NC |
| 33 | [4-Aminomethyl-phenylacetyl](1c)*SE(2c)WQ[Dab](2c)[Y(2-aminoethoxy)][2- |
| | Nal][2-Me-Leu]E(1c)Q[3-(3-Pyridyl)-Ala]-[NHMe] |
| | CNC(═O)[C@H](Cc1cccnc1)NC(═O)[C@H](CCC(N)═O)NC(═O)[C@@H]1CC |
| | C(═O)NCc2ccc(cc2)CC(═O)N[C@@H](CO)C(═O)N[C@H]2CCC(═O)NCC |
| | [C@H](NC(═O)[C@H](CCC(N)═O)NC(═O)[C@H](Cc3c[nH]c4ccccc34)NC2═O) |
| | C(═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 34 | [4-Aminomethyl-phenylacetyl](1c)*SE(2c)WQ[Dab](2c)[Y(2-aminoethoxy)][2- |
| | Nal][2-Me-Leu]E(1c)H[3-(3-Pyridyl)-Ala]-[NHMe] |
| | CNC(═O)[C@H](Cc1cccnc1)NC(═O)[C@H](Cc1cnc[nH]1)NC(═O)[C@@H]1 |
| | CCC(═O)NCc2ccc(cc2)CC(═O)N[C@@H](CO)C(═O)N[C@H]2CCC(═O)NCC |
| | [C@H](NC(═O)[C@H](CCC(N)═O)NC(═O)[C@H](Cc3c[nH]c4ccccc34)NC2═O) |
| | C(═O)N[C@@H](Cc2ccc(OCCN)cc2)C(═O)N[C@@H](Cc2ccc3ccccc3c2) |
| 35 | [4-Aminomethyl-phenylacetyl](1c)*SE(2c)WQ[Dab](2c)[Y(2-aminoethoxy)][2- |
| | Nal][2-Me-Leu]E(1c)[THP][3-(3-Pyridyl)-Ala]-[NHMe] |
| | CNC(═O)[C@H](Cc1cccnc1)NC(═O)C1(NC(═O)[C@@H]2CCC(═O)NCc3ccc |
| | (cc3)CC(═O)N[C@@H](CO)C(═O)N[C@H]3CCC(═O)NCC[C@H](NC(═O) |
| | [C@H](CCC(N)═O)NC(═O)[C@H](Cc4c[nH]c5ccccc45)NC3═O)C(═O)N[C@@ |
| | H](Cc3ccc(OCCN)cc3)C(═O)N[C@@H](Cc3ccc4ccccc4c3)C(═O)N[C@@] |
| | (C)(CC(C)C)C(═O)N2)CCOCC1 |
| 36 | [4-Aminomethyl-phenylacetyl](1c)*SE(2c)WQ[Dab](2c)[Y(2-aminoethoxy)][2- |
| | Nal][2-Me-Leu]E(1c)[hSer][3-(3-Pyridyl)-Ala]-[NHMe] |
| | CNC(═O)[C@H](Cc1cccnc1)NC(═O)[C@H](CCO)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | [C@@(C)(CC(C)C)C(═O)N1 |
| 37 | [4-Aminomethyl-phenylacetyl](1c)*SE(2c)WQ[Dab](2c)[Y(2-aminoethoxy)][2- |
| | Nal][2-Me-Leu]E(1c)[Ser(OMe)][3-(3-Pyridyl)-Ala]-[NHMe] |
| | CNC(═O)[C@H](Cc1cccnc1)NC(═O)[C@H](COC)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | [C@@](C)(CC(C)C)C(═O)N1 |
| 38 | [4-Aminomethyl-phenylacetyl](1c)*SE(2c)WQ[Dab](2c)[Y(2-aminoethoxy)][2- |
| | Nal][2-Me-Leu]E(1c)[{d}S][3-(3-Pyridyl)-Ala]-[NHMe] |
| | CNC(═O)[C@H](Cc1cccnc1)NC(═O)[C@@H](CO)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | [C@@](C)(CC(C)C)C(═O)N1 |
| 39 | [4-Aminomethyl-phenylacetyl](1c)*SE(2c)WQ[Dab](2c)[Y(2-aminoethoxy)][2- |
| | Nal][2-Me-Leu]E(1c)[2-Me-Ser][3-(3-Pyridyl)-Ala]-[NHMe] |
| | CNC(═O)[C@H](Cc1cccnc1)NC(═O)[C@](C)(CO)NC(═O)[C@@H]1CCC(═O) |
| | NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O) |
| | N[C@@H](Cc2ccc(OCCN)cc2)C(═O)N[C@@H](Cc2ccc3ccccc3c2)C(═O)N |
| | [C@@](C)(CC(C)C)C(═O)N1 |
| 40 | [4-Aminomethyl-phenylacetyl](1c)*AE(2c)WQ[Dab](2c)[Y(2-aminoethoxy)][2- |
| | Nal][2-Me-Leu]E(1c)S[3-(3-Pyridyl)-Ala]-[NHMe] |
| | CNC(═O)[C@H](Cc1cccnc1)NC(═O)[C@H](CO)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](Cc2ccc3ccccc3c2)C(═O)N |
| | [C@@](C)(CC(C)C)C(═O)N1 |
| 41 | [4-Aminomethyl-phenylacetyl](1c)*AE(2c)WQ[Dab](2c)[hPhe] |
| | [2-Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](CCc2ccccc2)C(═O)N[C@@H](Cc2ccc3ccccc3c2)C(═O)NC(C)(C)C |
| | (═O)N1 |
| 42 | [4-Aminomethyl-phenylacetyl](1c)*AE(2c)WQ[Dab](2c)[Y(2- |
| | aminoethoxy)][Cyclopropyl-Ala][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](CC2CC2)C(═O)NC(C)(C)C |
| | (═O)N1 |
| 43 | [4-Aminomethyl-phenylacetyl](1c)*AE(2c)WQ[Dab](2c)[7-AzaTrp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2c[nH]c3ncccc23)C(═O)N[C@@H](Cc2ccc3ccccc3c2)C(═O)NC |
| | (C)(C)C(═O)N1 |
| 44 | [4-Aminomethyl-phenylacetyl](1c)*AE(2c)WQ[Dab](2c)[Y(2-aminoethoxy)][7- |
| | AzaTrp][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](Cc2c[nH]c3ncccc23)C(═O)NC |
| | (C)(C)C(═O)N1 |
| 45 | [4-Aminomethyl-phenylacetyl](1c)*AE(2c)WQ[Dab](2c)[beta-hTrp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2c[nH]c3ccccc23)CC(═O)N[C@@H](Cc2ccc3ccccc3c2)C(═O)NC |
| | (C)(C)C(═O)N1 |
| 46 | [4-Aminomethyl-phenylacetyl](1c)*AE(2c)WQ[Dab](2c)[Y(2- |
| | aminoethoxy)][beta-hTrp][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](Cc2c[nH]c3ccccc23)CC(═O)N |
| | C(C)(C)C(═O)N1 |
| 47 | [4-Aminomethyl-phenylacetyl](1c)*AE(2c)WQ[Dab](2c)[7-F-Trp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2c[nH]c3c(F)cccc23)C(═O)N[C@@H](Cc2ccc3ccccc3c2)C(═O)N |
| | C(C)(C)C(═O)N1 |
| 48 | [4-aminomethyl-phenylacetyl(1c)*][N—Me-Ala]E(2c)WQ[Dab](2c)[Y(2- |
| | aminoethoxy)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N(C)[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N |
| | C(C)(C)C(═O)N1 |
| 49 | 4-aminomethyl-phenylacetyl](1c)*AE(2c)WQ[Dab](2c)[Y(2-aminoethoxy)][3- |
| | quinolinylalanine][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(OCCN)cc2)C(═O)N[C@@H](Cc2cnc3ccccc3c2)C(═O)NC |
| | (C)(C)C(═O)N1 |
| 51 | [4-aminomethyl-phenylacetyl](1c)*AE(2c)WQ[Dab](2c)[F(4- |
| | morpholine)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2ccc(N3CCOCC3)cc2)C(═O)N[C@@H](Cc2ccc3ccccc3c2)C(═O) |
| | NC(C)(C)C(═O)N1 |
| 52 | [4-aminomethyl-phenylacetyl](1c)*AE(2c)WQ[Dab](2c)[3- |
| | quinolinylalanine][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O)N |
| | [C@@H](Cc2cnc3ccccc3c2)C(═O)N[C@@H](Cc2ccc3ccccc3c2)C(═O)NC(C) |
| | (C)C(═O)N1 |
| 53 | [4-aminomethyl-phenylacetyl](1c)*AE(2c)WQ[Dab](2c)[F(4- |
| | morpholine)][2Nal][Aib]E(1c)[Ser(OCH3)][3-(3-Pyridyl)-alanyl]-[NHMe] |
| | CNC(═O)[C@H](Cc1cccnc1)NC(═O)[C@H](COC)NC(═O)[C@@H]1CCC(═O) |
| | NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C(═O) |
| | N[C@@H](Cc2ccc(N3CCOCC3)cc2)C(═O)N[C@@H](Cc2ccc3ccccc3c2)C(═O) |
| | NC(C)(C)C(═O)N1 |
| 54 | [4-aminomethyl-phenylacetyl](1c)*[3-Aminopropanoyl]E(2c)[7-Me- |
| | Trp][Q(2Me)][Dab](2c)[Y(2-aminoethoxy)(N(Me)2)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)NCCC(═O)N[C@H]2CCC(═O)NCC[C@H](NC(═O)[C@H] |
| | (CCC(═O)N(C)C)NC(═O)[C@H](Cc3c[nH]c4c(C)cccc34)NC2═O)C(═O)N[C@@H] |
| | (Cc2ccc(OCCN(C)C)cc2)C(═O)N[C@@H](Cc2ccc3ccccc3c2)C(═O)NC(C)(C)C(═O)N1 |
| 55 | [4-aminomethyl-phenylacetyl](1c)*[3-Aminopropanoyl]E(2c)[7-Me- |
| | Trp][Q(2Me)][Dab](2c)[Y(nPentylamine)(N+(Me)3)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)NCCC(═O)N[C@H]2CCC(═O)NCC[C@H](NC(═O) |
| | [C@H](CCC(═O)N(C)C)NC(═O)[C@H](Cc3c[nH]c4c(C)cccc34)NC2═O)C(═O)N |
| | [C@@H](Cc2ccc(OCCCCC[N+](C)(C)C)cc2)C(═O)N[C@@H](Cc2ccc3ccccc3c2) |
| | C(═O)NC(C)(C)C(═O)N1 |
| 56 | [4-aminomethyl-phenylacetyl](1c)*[3-Aminopropanoyl]E(2c)[7-Me- |
| | Trp][Q(2Me)][Dab](2c)[Tyr(2-trimethyl-PEG2)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)NCCC(═O)N[C@H]2CCC(═O)NCC[C@H](NC(═O) |
| | [C@H](CCC(═O)N(C)C)NC(═O)[C@H](Cc3c[nH]c4c(C)cccc34)NC2═O)C(═O)N |
| | [C@@H](Cc2ccc(OCCOC- |
| | COCC[N+](C)(C)C)cc2)C(═O)N[C@@H](Cc2ccc3ccccc3c2)C(═O)NC(C)(C)C |
| | (═O)N1 |
| 57 | [4-aminomethyl-phenylacetyl](1c)*[3-Aminopropanoyl]E(2c)[7-Me- |
| | Trp][K(NMePEG3)][Dab](2c)[Y(2- |
| | aminoethoxy)(N(Me)2)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC(═O) |
| | NCc2ccc(cc2)CC(═O)NCCC(═O)N[C@H]2CCC(═O)NCC[C@H](NC(═O) |
| | [C@H](CCCCN(C)C(═O)COC- |
| | COCC[N+](C)(C)C)NC(═O)[C@H](Cc3c[nH]c4c(C)cccc34)NC2═O)C(═O)N |
| | [C@@H](Cc2ccc(OCCN(C)C)cc2)C(═O)N[C@@H](Cc2ccc3ccccc3c2)C(═O)N |
| | C(C)(C)C(═O)N1 |

The bridging amino acid residue is the amino acid residue directly preceding the parentheses below. The square brackets indicate the bridging amino acid residues. For example, [2,11] is a bridge between amino acid residues 2 and 11. Similarly, [4,7] is a bridge between amino acid residues 4 and 7, and [1,11] is a bridge between amino acid residues 1 and 11.
* denotes that the (1c), (2a), (2c), and (4c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge; (4c) denotes the [1,11] lactam bridge.

The compound numbering of Table 1-1 corresponds to the same number as the SEQ ID NO., as shown in Table 1-1a below.

TABLE 1-1a
Sequence ID Numbers of synthesised compounds from Table 1-1

| SEQ ID NO: | Sequence |
|---|---|
| SEQ ID NO: 1 | [3-Aminopropanoyl](4c)*[3-(3-Pyridyl)-Ala]SC(2a)WQC(2a)[Y(2- |
| | aminoethoxy)][2-Nal][2-Me-Leu]E(4c)[{d}R] |
| SEQ ID NO: 2 | [3-(3-Pyridyl)-Ala](4c)*[3-Aminopropanoyl]SC(2a)WQC(2a)[Y(2- |
| | aminoethoxy)][2-Nal][2-Me-Leu]E(4c)[{d}R] |
| SEQ ID NO: 3 | [4-Aminomethyl-phenylacetyl](1c)*SE(2c)WQ[Dab](2c)[Y(2- |
| | aminoethoxy)][2-Nal][2-Me-Leu]E(1c)[Dab][2-Me-3-(3-Pyridyl)-Ala] |
| SEQ ID NO: 4 | [4-Aminomethyl-phenylacetyl](1c)*SE(2c)WQ[Dab](2c)[Y(2- |
| | aminoethoxy)][2-Nal][2-Me-Leu]E(1c)[Dab][N—Me-3-(3-Pyridyl)-Ala] |
| SEQ ID NO: 5 | [4-Aminomethyl-phenylacetyl](1c)*SE(2c)WQ[Dab](2c)[Y(2- |
| | aminoethoxy)][2-Nal][2-Me-Leu]E(1c)[Dab][{d}3-(3-Pyridyl)-Ala] |
| SEQ ID NO: 6 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 7 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 8 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 9 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][Sar] |
| SEQ ID NO: 10 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][{d}H] |
| SEQ ID NO: 11 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][beta-hLeu] |
| SEQ ID NO: 12 | [4-Aminomethyl-phenylacetyl](1c)*SE(2c)WQ[Dab](2c)[Y(2- |
| | aminoethoxy)][2-Nal][2-Me-Leu]E(1c)[Dab]H[3-(3-Pyridyl)-Ala] |
| SEQ ID NO: 13 | [4-Aminomethyl-phenylacetyl](1c)*SE(2c)WQ[Dab](2c)[Y(2- |
| | aminoethoxy)][2-Nal][2-Me-Leu]E(1c)[Dab][{d}H] |
| SEQ ID NO: 14 | [4-Aminomethyl-phenylacetyl](1c)*SE(2c)WQ[Dab](2c)[Y(2- |
| | aminoethoxy)][2-Nal][2-Me-Leu]E(1c)[Dab][H(Me)] |
| SEQ ID NO: 15 | [4-Aminomethyl-phenylacetyl](1c)*SE(2c)WQ[Dab](2c)[Y(2- |
| | aminoethoxy)][2-Nal][2-Me-Leu]E(1c)[Iso-Dab][2-Me-3-(3-Pyridyl)-Ala] |
| SEQ ID NO: 16 | [4-Aminomethyl-phenylacetyl](1c)*SE(2c)WQ[Dab](2c)[Y(2- |
| | aminoethoxy)][2-Nal][2-Me-Leu]E(1c)[{d}Gly-CF3][3-(3-Pyridyl)-Ala] |
| SEQ ID NO: 17 | [4-Aminomethyl-phenylacetyl](1c)*SE(2c)WQ[Dab](2c)[Y(2- |
| | aminoethoxy)][2-Nal][2-Me-Leu]E(1c)[Gly-CF3][3-(3-Pyridyl)-Ala] |
| SEQ ID NO: 18 | [4-Aminomethyl-phenylacetyl](1c)*[N—Me-Ser]E(2c)WQ[Dab](2c)[Y(2- |
| | aminoethoxy)][2-Nal][2-Me-Leu]E(1c)[Dab][3-(3-Pyridyl)-Ala] |
| SEQ ID NO: 19 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3,5-Pyrimidyl)-Ala] |
| SEQ ID NO: 20 | [4-Aminomethyl-phenylacetyl](1c)*AE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 21 | [4-Aminomethyl-phenylacetyl](1c)*FE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 22 | [4-Aminomethyl-phenylacetyl](1c)*SE(2c)WA[Dab](2c)[Y(2- |
| | aminoethoxy)][2-Nal][2-Me-Leu]E(1c)[Dab][3-(3-Pyridyl)-Ala] |
| SEQ ID NO: 23 | [4-Aminomethyl-phenylacetyl](1c)*SE(2c)WF[Dab](2c)[Y(2- |
| | aminoethoxy)][2-Nal][2-Me-Leu]E(1c)[Dab][3-(3-Pyridyl)-Ala] |
| SEQ ID NO: 24 | [4-Aminomethyl-phenylacetyl](1c)*SE(2c)W[K(Ac)][Dab](2c)[Y(2- |
| | aminoethoxy)][2-Nal][2-Me-Leu]E(1c)[Dab][3-(3-Pyridyl)-Ala] |
| SEQ ID NO: 25 | [4-Aminomethyl-phenylacetyl](1c)*SE(2c)W[Dab(Ac)][Dab](2c)[Y(2- |
| | aminoethoxy)][2-Nal][2-Me-Leu]E(1c)[Dab][3-(3-Pyridyl)-Ala] |
| SEQ ID NO: 26 | [4-Aminomethyl-phenylacetyl](1c)*SE(2c)W[Cit][Dab](2c)[Y(2- |
| | aminoethoxy)][2-Nal][2-Me-Leu]E(1c)[Dab][3-(3-Pyridyl)-Ala] |
| SEQ ID NO: 27 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab][3-(3-Pyridyl)-Ala] |
| SEQ ID NO: 28 | [4-Aminomethyl-phenylacetyl](1c)*SE(2c)W[Orn][Dab](2c)[Y(2- |
| | aminoethoxy)][2-Nal][2-Me-Leu]E(1c)[Dab][3-(3-Pyridyl)-Ala] |
| SEQ ID NO: 29 | [4-Aminomethyl-phenylacetyl](1c)*SE(2c)W[Q(Me)][Dab](2c)[Y(2- |
| | aminoethoxy)][2-Nal][2-Me-Leu]E(1c)[Dab][3-(3-Pyridyl)-Ala] |
| SEQ ID NO: 30 | [4-Aminomethyl-phenylacetyl](1c)*SE(2c)W[Q(2Me)][Dab](2c)[Y(2- |
| | aminoethoxy)][2-Nal][2-Me-Leu]E(1c)[Dab][3-(3-Pyridyl)-Ala] |
| SEQ ID NO: 31 | [4-Aminomethyl-phenylacetyl](1c)*SE(2c)WQ[Dab](2c)[Y(2- |
| | aminoethoxy)][2-Nal][2-Me-Leu]E(1c)S[3-(3-Pyridyl)-Ala] |
| SEQ ID NO: 32 | [4-Aminomethyl-phenylacetyl](1c)*SE(2c)WQ[Dab](2c)[Y(2- |
| | aminoethoxy)][2-Nal][2-Me-Leu]E(1c)[Nle][3-(3-Pyridyl)-Ala] |
| SEQ ID NO: 33 | [4-Aminomethyl-phenylacetyl](1c)*SE(2c)WQ[Dab](2c)[Y(2- |
| | aminoethoxy)][2-Nal][2-Me-Leu]E(1c)Q[3-(3-Pyridyl)-Ala] |
| SEQ ID NO: 34 | [4-Aminomethyl-phenylacetyl](1c)*SE(2c)WQ[Dab](2c)[Y(2- |
| | aminoethoxy)][2-Nal][2-Me-Leu]E(1c)H[3-(3-Pyridyl)-Ala] |
| SEQ ID NO: 35 | [4-Aminomethyl-phenylacetyl](1c)*SE(2c)WQ[Dab](2c)[Y(2- |
| | aminoethoxy)][2-Nal][2-Me-Leu]E(1c)[THP][3-(3-Pyridyl)-Ala] |
| SEQ ID NO: 36 | [4-Aminomethyl-phenylacetyl](1c)*SE(2c)WQ[Dab](2c)[Y(2- |
| | aminoethoxy)][2-Nal][2-Me-Leu]E(1c)[hSer][3-(3-Pyridyl)-Ala] |
| SEQ ID NO: 37 | [4-Aminomethyl-phenylacetyl](1c)*SE(2c)WQ[Dab](2c)[Y(2- |
| | aminoethoxy)][2-Nal][2-Me-Leu]E(1c)[Ser(OMe)][3-(3-Pyridyl)-Ala] |
| SEQ ID NO: 38 | [4-Aminomethyl-phenylacetyl](1c)*SE(2c)WQ[Dab](2c)[Y(2- |
| | aminoethoxy)][2-Nal][2-Me-Leu]E(1c)[{d}S][3-(3-Pyridyl)-Ala] |
| SEQ ID NO: 39 | [4-Aminomethyl-phenylacetyl](1c)*SE(2c)WQ[Dab](2c)[Y(2- |
| | aminoethoxy)][2-Nal][2-Me-Leu]E(1c)[2-Me-Ser][3-(3-Pyridyl)-Ala] |
| SEQ ID NO: 40 | [4-Aminomethyl-phenylacetyl](1c)*AE(2c)WQ[Dab](2c)[Y(2- |
| | aminoethoxy)][2-Nal][2-Me-Leu]E(1c)S[3-(3-Pyridyl)-Ala] |
| SEQ ID NO: 41 | [4-Aminomethyl-phenylacetyl](1c)*AE(2c)WQ[Dab](2c)[hPhe][2- |
| | Nal][Aib]E(1c)[Dab][3-(3-Pyridyl)-Ala] |
| SEQ ID NO: 42 | [4-Aminomethyl-phenylacetyl](1c)*AE(2c)WQ[Dab](2c)[Y(2- |
| | aminoethoxy)][Cyclopropyl-Ala][Aib]E(1c)[Dab][3-(3-Pyridyl)-Ala] |
| SEQ ID NO: 43 | [4-Aminomethyl-phenylacetyl](1c)*AE(2c)WQ[Dab](2c)[7-AzaTrp][2- |
| | Nal][Aib]E(1c)[Dab][3-(3-Pyridyl)-Ala] |
| SEQ ID NO: 44 | [4-Aminomethyl-phenylacetyl](1c)*AE(2c)WQ[Dab](2c)[Y(2- |
| | aminoethoxy)][7-AzaTrp][Aib]E(1c)[Dab][3-(3-Pyridyl)-Ala] |
| SEQ ID NO: 45 | [4-Aminomethyl-phenylacetyl](1c)*AE(2c)WQ[Dab](2c)[beta-hTrp][2- |
| | Nal][Aib]E(1c)[Dab][3-(3-Pyridyl)-Ala] |
| SEQ ID NO: 46 | [4-Aminomethyl-phenylacetyl](1c)*AE(2c)WQ[Dab](2c)[Y(2- |
| | aminoethoxy)][beta-hTrp][Aib]E(1c)[Dab][3-(3-Pyridyl)-Ala] |
| SEQ ID NO: 47 | [4-Aminomethyl-phenylacetyl](1c)*AE(2c)WQ[Dab](2c)[7-F-Trp][2- |
| | Nal][Aib]E(1c)[Dab][3-(3-Pyridyl)-Ala] |
| SEQ ID NO: 48 | [4-aminomethyl-phenylacetyl(1c)*][N—Me-Ala]E(2c)WQ[Dab](2c)[Y(2- |
| | aminoethoxy)][2Nal][Aib]E(1c)[Dab][3-(3-Pyridyl)-alanyl] |
| SEQ ID NO: 49 | 4-aminomethyl-phenylacetyl](1c)*AE(2c)WQ[Dab](2c)[Y(2- |
| | aminoethoxy)][3-quinolinylalanine][Aib]E(1c)[Dab][3-(3-Pyridyl)-alanyl] |
| SEQ ID NO: 51 | [4-aminomethyl-phenylacetyl](1c)*AE(2c)WQ[Dab](2c)[F(4- |
| | morpholine)][2Nal][Aib]E(1c)[Dab][3-(3-Pyridyl)-alanyl] |
| SEQ ID NO: 52 | [4-aminomethyl-phenylacetyl](1c)*AE(2c)WQ[Dab](2c)[3- |
| | quinolinylalanine][2Nal][Aib]E(1c)[Dab][3-(3-Pyridyl)-alanyl] |
| SEQ ID NO: 53 | [4-aminomethyl-phenylacetyl](1c)*AE(2c)WQ[Dab](2c)[F(4- |
| | morpholine)][2Nal][Aib]E(1c)[Ser(OCH3)][3-(3-Pyridyl)-alanyl] |
| SEQ ID NO: 54 | [4-aminomethyl-phenylacetyl](1c)*[3-Aminopropanoyl]E(2c)[7-Me- |
| | Trp][Q(2Me)][Dab](2c)[Y(2-aminoethoxy)(N(Me)2)][2Nal][Aib]E(1c)[Dab][3- |
| | (3-Pyridyl)-alanyl] |
| SEQ ID NO: 55 | [4-aminomethyl-phenylacetyl](1c)*[3-Aminopropanoyl]E(2c)[7-Me- |
| | Trp][Q(2Me)][Dab](2c)[Y(nPentylamine)(N⁺(Me)3)][2Nal][Aib]E(1c)[Dab][3- |
| | (3-Pyridyl)-alanyl] |
| SEQ ID NO: 56 | [4-aminomethyl-phenylacetyl](1c)*[3-Aminopropanoyl]E(2c)[7-Me- |
| | Trp][Q(2Me)][Dab](2c)[Tyr(2-trimethyl-PEG2)][2Nal][Aib]E(1c)[Dab][3-(3- |
| | Pyridyl)-alanyl] |
| SEQ ID NO: 57 | [4-aminomethyl-phenylacetyl](1c)*[3-Aminopropanoyl]E(2c)[7-Me- |
| | Trp][K(NMePEG3)][Dab](2c)[Y(2- |
| | aminoethoxy)(N(Me)2)][2Nal][Aib]E(1c)[Dab][3-(3-Pyridyl)-alanyl] |

*denotes that the (1c), (2a), (2c), and (4c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge; (4c) denotes the [1,11] lactam bridge.

For comparison purposes, four compounds from Kong et al., 2020 having two 1,3-dithio-propan-2-one bridges were synthesised (Table 1-2).

TABLE 1-2
Synthesised compounds from Kong et al., 2020

| Compound | Structure |
|---|---|
| I1 (isomer 3) | H-GC(1a)TC(2a)WEC(2a)WWLC(1a)S-[NH2] |
| I3 (isomer 3) | H-DC(1a)SC(2a)WQC(2a)WWLC(1a)R-[NH2] |
| I4 (isomer 3) | H-EC(4a)WLC(5a)YYRNC(5a)NC(4a)M-[NH2] |
| I5 (isomer 3) | H-GC(1a)TC(2a)WEC(2a)WW[2-Me-Leu]C(1a)S-[NH2] |

(1a) denotes the [2,11] 1,3-dithio-propan-2-one bridge; (2a) denotes the [4,7] 1,3-dithio-propan-2-one bridge; (4a) denotes the [1,11] 1,3-dithio-propan-2-one bridge; (5a) denotes the [4,9] 1,3-dithio-propan-2-one bridge.

Additionally, two compounds from the Protagonist patent applications (Compound C in WO 2016/011208, WO 2017/011820, and Sayago et al., 2018; and Peptide 993 in WO 2018/089693) having a bridging cystathionine amino acid residue at [2,7] was synthesised (Table 1-3).

TABLE 1-3
Synthesised compound from the Protagonist patent applications

| Compound | Structure |
|---|---|
| Compound C | [Ac]-[Abu](16i)QTWQC(16i)[Y(2-aminoethoxy)]W[2-Me-Lys]ENG-[NH2] |
| Peptide 993 | [Ac]-[{d}R][Abu](16i)QTWQC(16i)[Y(2-aminoethoxy)][2-Nal][4- |
| | Aminotetrahydro-2H-pyran-4-carbonyl]ENN-[NH2] |

(16i) denotes the [2,7] thioether bridge. The combination of Abu at position X2, Cys at position X7, and the [2,7] thioether bridge forms a cystathionine as the bridging amino acid residue.

Other reference compounds synthesised are described in Table 1-4. These reference compounds were disclosed in WO2023/099669.

TABLE 1-4
Synthesised reference compounds

| Compound | Structure |
|---|---|
| Ref 4 | H-DE(1c)SC(2a)WQC(2a)WWLK(1c)R-[NH2] |
| Ref 17 | [Ac]-DE(1c)SC(2a)WQC(2a)W[2-Nal]LK(1c)R-[NH2] |
| Ref 18 | [Ac]-K(1c)SC(2a)WQC(2a)W[2-Nal]LE(1c)R-[NH2] |
| Ref 37 | [(3-Aminomethyl)benzoyl](1c)*SC(2a)WQC(2a)W[2-Nal]LE(1c)R-[NH2] |
| Ref 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N—Me-Ser]C(2a)WQC(2a)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 155 | [Ac]-K(1c)TC(2a)WQC(2a)[Y(2-aminoethoxy)][2-Nal][2-Me- |
| | Leu]E(1c)[Dab][3-(3-Pyridyl)-Ala]-[NH2] |

(1c) denotes the [2,11] lactam bridge; (2a) denotes the [4,7] 1,3-dithio-propan-2-one bridge; (2c) denotes the [4,7] lactam bridge, and
* denotes that the (1c), (2a), and (2c) bridges use the peptide backbone amine or carboxylic acid at the N- or C-terminus, not the side chain amine or carboxylic acid.

Unless otherwise specified, reagents and solvents employed in the following were available commercially in standard laboratory reagent or analytical grade, and were used without further purification.

### Apparatus and Synthetic Strategy

Peptides were synthesized batchwise on a peptide synthesiser, such as a CEM Liberty Blue Peptide Synthesizer, according to solid phase peptide synthetic procedures using 9-fluorenylmethyloxycarbonyl (Fmoc) as N-α-amino protecting group and suitable common protection groups for side chain functionalities.

As polymeric support based resins, such as e.g. TentaGel™, was used. The synthesizer was loaded with resin that prior to usage was swelled in DMF.

Non-naturally occurring amino acids and other suitable building blocks were employed without any changes to the general procedure.

Optical isomers of particular amino acids (including non-naturally occurring amino acids) were employed in the synthesis of the compounds and may be found in Tables A and B in the definitions. Definitions of particular R² groups may be found in Table C in the definitions.

### Coupling on a CEM Liberty Blue Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 50° C. and coupled for 10 minutes or coupled with no heat for 60 minutes. During the coupling nitrogen was bubbled through the mixture.

In the case of difficult couplings (e.g. coupling of a residue immediately after an N-methylated amino acid residue or other sterically hindered amino acid residue as recognized by a person of skill in the art) the coupling was repeated one or more times.

### Deprotection:

The Fmoc group was deprotected using piperidine in DMF or other suitable solvents. The deprotection solution was added to the reaction vessel and the mixture was heated for 5 minutes reaching approx. 50° C. After draining the reaction vessel the resin was washed with DMF or other suitable solvents.

### Lactam Formation:

The following procedure for the coupling of Glu and Lys is representative for all lactam formations where the amino acid side chain containing the carboxyl-function is protected with Oall and the amino acid side chain containing the amino group is Alloc-protected. After assembly of the full peptide sequence, deprotection of Glu(Oall) and Lys(Alloc) was performed using Pd(PPh₃)₄ (0.05 equiv.) and PhSiH₃ (10 equiv.) in DCM. Subsequently, the lactam bridge was formed between the side chain carboxylic acid of Glu and side chain amine of Lys using PyBOP (2 equiv.) and DIPEA (3.0 equiv.) in DMF. Both steps were performed with the peptide still attached to the resin.

Peptides with a lactam from a side chain to the N-terminal amine were prepared similarly. After assembly of the full peptide sequence, the Fmoc protection group of the N-terminal amine is left intact. Glu(Oall) is deprotected as described with Pd(PPh₃)₄ followed by a Fmoc deprotection (see section “cleavage”). The lactam bridge was formed similarly with PyBOP.

### Cleavage:

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide:

The crude peptide was purified by preparative reverse phase HPLC using a conventional HPLC apparatus, such as a Gilson GX-281 with 331/332 pump combination, for binary gradient application equipped with a column, such as 5×25 cm Gemini NX 5u C18 110A column, and a fraction collector using a flow 20-40 ml/min with a suitable gradient of buffer A (0.1% Formic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Dithioether Formation:

The methodology for dithioether formation from the reaction between two unprotected cysteines and a linker can be performed as previously described in Kong et al., 2020, or through the modified method below.

The crude intermediate peptide was dissolved in 3:7 mixture of water/acetonitrile (1 mg/mL). DODT (2 equiv.) was added and the mixture was stirred at room temperature for 10 min. The pH was adjusted to pH 8 by addition of 0.2 M ammonium carbonate. The linker (2 equiv.) was added directly to the solution and the mixture was left shaken at room temperature over night. The solution was filtered with 0.45 μm filter and loaded directly on a prep-HPLC column for a final purification.

Linkers used in the synthesized compounds include:
1,3-dibromopropan-2-one, CAS #816-39-7.

### Gly-CF3 and D-Gly-CF3:

The di-amino acid “Fmoc-Gly-CF3-3Pal-OH” and “Fmoc-D-Gly-CF3-3Pal-OH” was synthesized as described in: Solid-Phase Synthesis of Gly-4Ψ[CH(CF3)NH]-Peptides, Sgorbati et al., J. Org. Chem, 2021, 86, 9225-9232, using the same synthesis procedure as for “Fmoc-Gly-CF3-Phe-OH” (that is, compound 4c as shown in Table 1 following hydrolysis step (i) in Scheme 1b). These di-amino acids were utilized in the synthesis of, for example, compounds 16 and 17.

### Iso-Amino Acids:

Peptides with iso-amino acids were synthesized by standard Fmoc SPPS, using amino acid building blocks where the protecting groups at the N-terminal and side chain are switched or interchanged. For example, a peptide with a standard Lysine in its sequence is synthesised with the building block Fmoc-Lys(Boc)-OH, where Fmoc is the protecting group at the N-terminal amine and Boc at the side chain. In contrast, a peptide with iso-Lys would thus be synthesised with a building block of Boc-Lys(Fmoc)-OH instead of Fmoc-Lys(Boc)-OH. Coupling conditions for attaching the building block to the peptide and subsequent deprotecting conditions for removal of the Fmoc group are the same as described for standard Fmoc SPPS synthesis.

The side-chain protecting group “Boc” may be replaced with an “Alloc” protecting group in cases where side chain on-resin modifications are performed.

### Analytical HPLC:

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100×4.6 mm column. The compound was detected and quantified at 215 nm. Buffers A (0.1% TFA, aq.) and buffer B (0.1% TFA, 90% MeCN, aq.).

### Mass Spectroscopy:

Final MS analysis was performed on a conventional mass spectrometer, e.g. Waters Xevo G2 Tof, equipped with electrospray detector with lock-mass calibration and MassLynx software. It was operated in positive mode using direct injection and a cone voltage of 15V (1 TOF), 30 V (2 TOF) or 45 V (3 TOF) as specified on the chromatogram. Precision was 5 ppm with a typical resolution of 15,000-20,000.

One of skill in the art will appreciate that standard methods of peptide synthesis may be used to generate the compounds of the invention.

### Example 2: Structure-Activity-Relationship (SAR) of the Compounds

The SAR from the peptides I1 (isomer 3), I3 (isomer 3), I4 (isomer 3), and I5 (isomer 3) from Kong et al., 2020 and the compounds disclosed in WO2023/099669 are described in Example 2 of WO2023/099669, which is incorporated by reference.

The inventors identified Ref 122 as a favourable compound from WO2023/099669 (compound 122 of WO2023/099669):

Ref 122 Amino Acid Sequence

| N | 1 | 2 | 3 | 4 | 5 | 6 | 7 | 8 | 9 | 10 | 11 | 12 | 13 | 14 | C |
|---|---|---|---|---|---|---|---|---|---|---|---|---|---|---|---|
| — | — | α | S | E | W | Q | Dab | β | 2-Nal | γ | E | Dab | 3-Pal | — | NH2 |
| | | (1c) | | (2c) | | | (2c) | | | | (1c) | | | | |

α = 4-aminomethyl-phenylacetyl; β = Y(2-aminoethoxy); γ = 2-Me-Leu; (1c) = [2,11] lactam bridge; (2c) = [4,7] lactam bridge.

Ref 122 was potent (Kᵢ hIL-23R of 0.00541 nM, equivalent to IC₅₀ (hIL-23R) of 0.0066 nM, and IC₅₀ (pSTAT3) of 0.028 nM—see Examples 3-1 and 3-2, Tables 3-1 and 3-2 below). However, Ref 122 was not stable under the SIF assay (only 25% of intact Ref 122 was remaining in the SIF assay after 4 h—see Example 3-3, Table 3-3 below) meaning it would not be sufficiently stable in the gastrointestinal tract.

The inventors identified a major metabolite formed after incubation of Ref 122 in the SIF assay which included the peptide without the C-terminal 3Pal (3-(3-Pyridyl)-Ala) amino acid.

Based on this insight and using Ref 122 as the peptide starting point the inventors thus tried four different approaches to improve SIF by addressing the C-terminus:
(i) Changing the C-terminal amino acid to a D-isomer amino acid;
(ii) Methylating the C-terminal amino acid;
(iii) Adding an unnatural amino acid at the C-terminus; and
(iv) Derivatisation of the C-terminus.
(i) Changing the C-terminal amino acid to a D-isomer amino acid

The inventors changed the C-terminal 3-Pal in Ref 122 to D-3-Pal and D-His. The D-His and D-3-Pal modifications moderately increased SIF stability to 41% and 56% of intact peptide remaining after 4 h (Table 2-1). However, both modifications slightly decreased the potency of the compound.

TABLE 2-1
IC₅₀ and SIF data for D-isomer amino acids

| | | | | IC₅₀ | | SIF |
|---|---|---|---|---|---|---|
| | | | | (hIL-23R | IC₅₀ | (% |
| | | | | binding) | (pSTAT3) | after |
| | 13 | 14 | C | (nM) | (nM) | 4 h) |
| Ref 122 | 3-Pal | — | NH2 | 0.0066 | 0.028 | 25 |
| Compound 5 | {d}3-Pal | — | NH2 | 0.093 | 2.1 | 41 |
| Compound 13 | {d}H | — | NH2 | 0.059 | 6.8 | 56 |

All other amino acid residues are the same in the above compounds.

### (ii) Methylating the C-Terminal Amino Acid

The inventors also methylated the C-terminal 3-Pal in Ref 122 to prevent cleavage. The 2-position of 3-Pal was methylated (2-Me-3-Pal), the amine of 3-Pal was methylated (N-Me-3-Pal), and the C-terminal amide was methylated (C═NHMe).

All three modifications greatly improved SIF stability to above 80% of intact peptide remaining after 4 h (Table 2-2). The 2-Me-3-Pal and N-Me-3-Pal slightly decreased the potency of the compound. However, the methylation of the C-terminal amide retained potency. Thus, methylation at the C-terminus improved SIF stability whilst retaining potency of the compound.

TABLE 2-2
IC₅₀ and SIF data for methylated C-terminal amino acid

| | | | | IC₅₀ | | SIF |
|---|---|---|---|---|---|---|
| | | | | (hIL-23R | IC₅₀ | (% |
| | | | | binding) | (pSTAT3) | after |
| | 13 | 14 | C | (nM) | (nM) | 4 h) |
| Ref 122 | 3-Pal | — | NH2 | 0.0066 | 0.028 | 25 |
| Compound 3 | α | — | NH2 | 0.034 | 1.7 | 109 |
| Compound 4 | β | — | NH2 | 0.17 | 6.5 | 89 |
| Compound 8 | 3-Pal | — | NHMe | 0.0098 | 0.053 | 105 |

α = 2-Me-3-Pal; β = N-Me-3-Pal
All other amino acid residues are the same in the above compounds.

(iii) Adding an Unnatural Amino Acid at the C-Terminus

The inventors also tested if adding an additional unnatural amino acid residue at the C-terminus would prevent the 3-Pal cleavage in Ref 122. The addition of sarcosine (Sar), D-histidine ({d}H), and beta-homo-Leu at position 14 were tested.

The addition of D-histidine did not increase SIF stability, but the modification did retain potency of the compound (Table 2-3).

Nevertheless, the addition of Sar and beta-homo-Leu greatly improved SIF stability to above 102% of intact peptide remaining after 4 h (Table 2-3). The addition of beta-homo-Leu slightly decreased the potency of the compound, but the addition of Sar maintained the potency of the compound.

TABLE 2-3
IC₅₀ and SIF data for additional unnatural amino acid

| | | | | IC₅₀ | | SIF |
|---|---|---|---|---|---|---|
| | | | | (hIL-23R | IC₅₀ | (% |
| | | | | binding) | (pSTAT3) | after |
| | 13 | 14 | C | (nM) | (nM) | 4 h) |
| Ref 122 | 3-Pal | — | NH2 | 0.0066 | 0.028 | 25 |
| Compound 9 | 3-Pal | Sar | NH2 | 0.0053 | 0.025 | 105 |
| Compound 10 | 3-Pal | {d}H | NH2 | 0.0058 | 0.038 | 11 |
| Compound 11 | 3-Pal | γ | NH2 | 0.024 | 0.68 | 102 |

γ = beta-homo-Leu
All other amino acid residues are the same in the above compounds.

### (iv) Derivatisation of the C-Terminus

Finally, the inventors also tested if removing the 3-Pal and derivatising the group at the C-terminus would prevent degradation of Ref 122. An alkyl group substituted with a pyridyl group was used to derivatise the C-terminus.

Both of the tested C-terminal derivatives greatly improved SIF stability and prevented the degradation of the intact peptide remaining after 4 h (Table 2-4). The C-terminal derivatives slightly reduced the potency of the compound, with Compound 7 comprising the shorter derivative “NH-(2-(pyridin-3-yl)ethyl)” being more potent than Compound 6 with the longer derivative “NH-(4-(pyridin-3-yl)butanyl)”.

TABLE 2-4
IC₅₀ and SIF data for the derivatisation of the C-terminus

| | | | | IC₅₀ | | SIF |
|---|---|---|---|---|---|---|
| | | | | (hIL-23R | IC₅₀ | (% |
| | | | | binding) | (pSTAT3) | after |
| | 13 | 14 | C | (nM) | (nM) | 4 h) |
| Ref 122 | 3-Pal | — | NH2 | 0.0066 | 0.028 | 25 |
| Compound 6 | — | — | δ | 0.037 | 3.2 | 73 |
| Compound 7 | — | — | ε | 0.012 | 0.13 | 130 |

δ = NH-(4-(pyridin-3-yl)butanyl); ε = NH-(2-(pyridin-3-yl)ethyl)
All other amino acid residues are the same in the above compounds.

In conclusion, the inventors have found four ways to improve the SIF stability of the compounds disclosed herein. The above modifications can be used alone or in combination to improve the SIF stability of the compounds, and in many cases also retain or improve the potency of the compounds against IL-23R.

In particular, the inventors identified Compound 8 from (ii), Compound 9 from (iii), and Compound 7 from (iv) as particularly potent and SIF stable compounds.

### Example 3: Biological Assays

### Example 3-1: Binding Assay for Estimating Binding Affinity of Compounds to Human IL-23R

The compound binding affinity for IL-23R was estimated by the ability of compounds to displace a fluorophore-labelled reference compound from the human IL-23R. The assay principle relies on Bioluminescence Resonance Energy Transfer (BRET) between a fluorophore-labelled reference compound binding to the IL-23R part of a fusion protein consisting of the IL-23R fused to a Nanoluc luciferase enzyme (Nanoluc). The Nanoluc is placed in the N-terminus in close proximity to the binding domain of the ligand. Upon close proximity between the fluorophore of the fluorophore-labelled compound and the Nanoluc of the fusion protein, bioluminescence energy generated from conversion of a Nanoluc substrate, is transferred to the fluorophore resulting in an increase in BRET. In presence of an unlabelled compound, the unlabelled compound displaces the fluorophore-labelled peptide from its binding site resulting in a decrease in BRET. The concentration for which half of the fluorophore-labelled peptide is displaced by the unlabelled compound depends on the affinity of the compound for the IL-23R, and is termed the IC₅₀ concentration.

The fusion protein was generated by subcloning of the cDNA encoding the mature human IL-23R (primary accession number UniProtKB—Q5VWK5, amino acids 22-629) and a small linker sequence in frame with a mammalian expression plasmid encoding a secretion signal and the Nanoluc protein (N1371, Promega). The plasmid also contained a gene to confer resistance for the antibiotic hygromycin. A cell line stably expressing the Nluc-IL23R fusion protein was generated by transfection of HEK293 cells with the expression plasmid and selected with hygromycin for 3 weeks in Growth medium consisting of DMEM w/Glutamax-I containing, 10% V/V FBS, 1% V/V P/S, 1 mM Sodium Pyruvate, and 1× NEAA, and 0.3 mg/mL hygromycin. The remaining cells were propagated and considered a stable expressing Nluc-IL23R fusion protein pool clone.

Cells expressing the Nluc-IL23R fusion protein were expanded in Growth medium and membranes prepared by homogenization of a cell pellet from 18 T175 flasks (4° C. in subsequent steps). The cell pellet was lysed Tris 10 mM, 7.5, 1 mM EDTA and protease inhibitors (Complete, Roche) and homogenized using a 15 mL glass-dounce by 50 strokes. The homogenate was spun at spin @1500 rpm for 10 min, the supernatant transferred to SV-34 tubes and spun at 40000 g for 20 min at 4° C. to pellet the crude membranes. The supernatant was then removed, the pellet resuspended in 5 mL buffer containing 50 mM HEPES pH 7.4, 5 mM EGTA, and 5 mM MgCl₂, and homogenized. Aliquots of the resuspended and homogenized membranes containing the Nluc-IL23R fusion protein were stored at −80° C. until use.

Compounds to be tested for binding to IL-23R were serially diluted in Assay buffer (50 mM HEPES pH 7.4, 5 mM EGTA, 5 mM MgCl₂, 0.005% Tween-20, and 0.05% casein) and added to the wells of a white 384-well plate (Corning 3572) in a volume of 6.25 μL, together with 12.5 μL diluted membranes containing the Nluc-IL23R fusion protein (0.42 μg/well) and 6.25 μL fluorescently labelled peptide to a final at a concentration of 3.1 nM, also prepared in Assay buffer. The plate was sealed with light impermeable plate seals, and incubated on an orbital shaker 400 rpm for 2 hours at room temperature. To determine the BRET ratio, the plate seal was removed and 25 μL of the Nanoluc substrate (Promega N1572) diluted 1:500 was added to each well and incubated for 1-2 minutes on an orbital shaker at 400 rpm. Then plate was read on an Envision plate reader equipped with a luminescence mirror module (barcode 404) using filters corresponding to Nanoluc substrate luminescence (M470 filter; 470 nm, bandwidth 24 nm) and TAMRA fluorescence (M595p filter 595 nm, bandwidth 60 nm). The BRET ratio was calculated as the fluorescence from the TAMRA/nanoluc bioluminescence.

For data analysis BRET ratios were normalized relative to the BRET signal by 3.1 nM TAMRA-labelled peptide alone (no unlabelled compound added) and the BRET signal under complete displacement (by adding a very high concentration of unlabelled peptide).

Compound potency (IC₅₀) and maximal displacement (% displacement) were estimated by computer-aided curve fitting using a 4-parameter logistic (4PL) non-linear model. The IC₅₀ and maximal for each compound was determined by computer-aided curve fitting using a 4-parameter logistic (4PL) non-linear model. Data for the compound potency (IC₅₀) and maximal displacement (% displacement) are shown in Table 3-1. Typically compounds with low IC₅₀ are desired. Typically compounds with a high degree of displacement of the TAMRA-labelled peptide are desired. Due to experimental errors in the particular assay, values equal to or above 90% are typically considered as capable of completely the TAMRA-labelled peptide.

In the previous application WO2023/099669, the data were provided as calculated Kᵢ values. In this application, the inventors provide the data as IC₅₀ values. The Cheng-Prussov equation describes the relationship between the Kᵢ and the IC₅₀ as: Kᵢ=IC₅₀/(1+[L_{L}]/K_{dL}), where [L_{L}] is the concentration of labelled compound used and K_{dL} is the equilibrium dissociation constant for the labelled compound (Cheng and Prusoff, 1973). To enable comparison between values, the inventors have determined Kᵢ and IC₅₀ for a number of compounds from WO2023/099669:

Kᵢ and Corresponding IC₅₀ Values for Compounds in WO2023/099669

| | | IC₅₀ (hIL-23R |
|---|---|---|
| Compound | Kᵢ hIL-23R (nM) | binding) (nM) |
| Ref 4 | 143 | 180 |
| Ref 17 | 70.7 | 87 |
| Ref 18 | 2.85 | 3.5 |
| Ref 37 | 9.87 | 12 |
| Ref 41 | 2.31 | 2.8 |
| Ref 52 | 0.0475 | 0.058 |
| Ref 78 | 0.0205 | 0.025 |
| Ref 80 | 0.150 | 0.18 |
| Ref 81 | 0.318 | 0.39 |
| Ref 82 | 0.0665 | 0.082 |
| Ref 120 | 0.00938 | 0.011 |
| Ref 122 | 0.00541 | 0.0066 |
| Ref 125 | 0.599 | 0.73 |
| Ref 141 | — | 3.5 |
| Ref 146 | 1.01 | 1.2 |
| Ref 155 | 0.00805 | 0.0099 |
| I1 (isomer 3) | 104 | 130 |
| I3 (isomer 3) | 38.0 | 47 |
| I4 (isomer 3) | Tested | Tested |
| I5 (isomer 3) | 73.6 | 90 |
| Compound C | 2.11 | 2.6 |

Tested = Kᵢ or IC₅₀ estimated higher than 1 μM when tested in assays

TABLE 3-1
IC₅₀ (hIL-23R binding) and % displacement
(hIL-23R binding) data

| | IC₅₀ (hIL-23R binding) | % displacement |
|---|---|---|
| Compound | (nM) | (hIL-23R binding) |
| 1 | 5.4 | 105 |
| 2 | 2.9 | 103 |
| 3 | 0.034 | 99 |
| 4 | 0.17 | 111 |
| 5 | 0.093 | 99 |
| 6 | 0.037 | 103 |
| 7 | 0.012 | 102 |
| 8 | 0.0098 | 103 |
| 9 | 0.0053 | 100 |
| 10 | 0.0058 | 91 |
| 11 | 0.024 | 103 |
| 12 | 0.057 | 104 |
| 13 | 0.059 | 100 |
| 14 | 0.11 | 102 |
| 15 | 0.45 | 96 |
| 16 | 0.13 | 101 |
| 17 | 1.2 | 98 |
| 18 | 0.015 | 96 |
| 19 | 0.054 | 103 |
| 20 | 0.012 | 97 |
| 21 | 0.014 | 95 |
| 22 | 0.035 | 96 |
| 23 | 0.12 | 97 |
| 24 | 0.014 | 97 |
| 25 | 0.011 | 93 |
| 26 | 0.015 | 95 |
| 27 | 0.017 | 97 |
| 28 | 0.011 | 97 |
| 29 | 0.011 | 96 |
| 30 | 0.018 | 104 |
| 31 | 0.012 | 97 |
| 32 | 0.027 | 98 |
| 33 | 0.014 | 92 |
| 34 | 0.013 | 90 |
| 35 | 0.039 | 101 |
| 36 | 0.014 | 94 |
| 37 | 0.013 | 95 |
| 38 | 0.066 | 102 |
| 39 | 0.024 | 99 |
| 40 | 0.019 | 100 |
| 41 | 0.28 | 104 |
| 42 | 12 | 100 |
| 43 | 0.048 | 101 |
| 44 | 0.27 | 100 |
| 45 | 4.2 | 104 |
| 46 | 57 | 101 |
| 47 | 0.069 | 103 |
| 48 | 0.010 | 96 |
| 49 | 0.023 | 89 |
| 51 | 0.073 | 105 |
| 52 | 0.081 | 102 |
| 53 | 0.23 | 102 |
| Ref 4 | 180 | 100 |
| Ref 17 | 87 | 101 |
| Ref 18 | 3.5 | 103 |
| Ref 37 | 12 | 103 |
| Ref 41 | 2.8 | 110 |
| Ref 52 | 0.058 | 100 |
| Ref 78 | 0.025 | 100 |
| Ref 80 | 0.18 | 98 |
| Ref 81 | 0.39 | 101 |
| Ref 82 | 0.082 | 103 |
| Ref 120 | 0.011 | 94 |
| Ref 122 | 0.0066 | 97 |
| Ref 125 | 0.73 | 101 |
| Ref 141 | 3.5 | 102 |
| Ref 146 | 1.2 | 105 |
| Ref 155 | 0.0099 | 97 |
| I1 (isomer 3) | 130 | 105 |
| I3 (isomer 3) | 47 | 101 |
| I4 (isomer 3) | Tested | 84 |
| I5 (isomer 3) | 90 | 102 |
| Compound C | 2.6 | 102 |
| Peptide 993 | 0.24 | 101 |

Tested = IC₅₀ estimated higher than 1 μM when tested in assays

### Example 3-2: Functional Inhibition of IL-23 Mediated STAT3 Signalling by Compounds

The ability of compounds to inhibit IL-23 mediated signalling was determined in the human-derived DB cell line (CRL-2289) (hereafter referred to as DB cells), which endogenously expresses the human IL-23R and human IL-12R 31 subunits. Upon binding of IL-23, the IL-23R forms a heterodimer signalling complex together with IL-12R β1, which through the JAK2/STAT3 pathway promotes phosphorylation of STAT3 to form phospho-STAT3. In the assay, the functional antagonism of IL-23 mediated phospho-STAT3 formation in DB cells by the compound is quantified using reagents capable of measuring the phosphorylation state of Tyr705 of STAT3 in the form of a phospho-STAT3 (Tyr705) MSD (Meso Scale Discovery) kit.

The assay was used to quantify the functional antagonism of a compound and to rank order the inhibitor compounds according to their potency. For compounds tested in this assay, the response was normalized relative to control values to calculate the IC₅₀ and maximal inhibitory response from a concentration response curve of the compound in presence of a fixed concentration of human IL-23.

The assay procedure was as follows. DB cells were maintained in growth medium consisting of RPMI-1640 [Invitrogen 61870-010] supplemented with 10% V/V Foetal Bovine Serum (FBS) [(heat inactivated), Invitrogen 10270-106], and 1% V/V Penicillin-Streptomycin (Pen-Strep) Solution [Invitrogen 15140]. On the day of assay, cells were resuspended in Assay buffer consisting of RPMI-1640 [Invitrogen 61870-010] supplemented with 0.1% w/V BSA [Sigma-Aldrich A9430] to a density of 7.5×10⁶ cells/mL. Compounds to be tested for inhibition of hIL-23 mediated signalling were serially diluted in Assay buffer to 3× the final concentration. A solution of hIL-23 corresponding to 3× the EC₈₀ of hIL-23 (1.7 nM) was also prepared in Assay buffer. To initiate the assay, 20 μL of DB cell suspension (corresponding to 150.000 cells/well) was added to the wells of a 96-well V-bottom Polypropylene plate [Corning 3363] followed by addition of 3×20 μL of the diluted test compounds to separate wells. Following preincubation of the DB cells with inhibitor for 15 min in a cell incubator (37° C., 5% CO₂), 20 μL of the prepared 3× the EC₈₀ of hIL-23 solution was added to each well and incubated for 90 min in a cell incubator (37° C., 5% CO₂). For some wells buffer only or IL-23 only corresponding to the EC₈₀ was added to obtain readouts needed for normalization. To terminate the assay, the plate was spun at 1000 G for 5 min to pellet cells, the supernatant removed using an 8-channel manual pipette, and then added 50 μL/well Complete Lysis Buffer from the MSD STAT3 kit (Cat #K150SVD, Mesoscale) to the cell pellet. To completely lyse cells to release phospho-STAT3 for detection, the plate was shaken for 10 min at room temperature (500 rpm) sealed with aluminium foil and placed at −80° C. for a minimum of 15 min. Detection of the phospho-STAT3 level in the cell lysate from the individual wells was determined using the MSD STAT3 kit (Cat #K150SVD, Mesoscale) and read on a Meso QuickPlex SQ 120 plate reader (Mesoscale).

For data analysis raw data counts from the Meso QuickPlex SQ 120 plate reader were normalized relative to the response by the EC₈₀ of hIL-23 alone (no compound added) and the buffer level. Compound potency (IC₅₀) and maximal inhibitory response (% inhibition) were estimated by computer-aided curve fitting using a 4-parameter logistic (4PL) non-linear model. Data for the compound potency (IC₅₀) and maximal inhibitory response (% displacement) are shown in Table 3-2. The lower the IC₅₀ value, the more potent the compound. Typically compounds with a low IC₅₀ are desired. Typically compounds capable of completely inhibiting the IL23-induced response are desired. Due to experimental errors in the particular assay, values equal to or above 95% are typically considered as capable of completely inhibiting IL23R-induced signaling.

TABLE 3-2
hIL-23 (pSTAT3) antagonist IC₅₀ and % inhibition data

| | hIL-23 (pSTAT3) | hIL-23 (pSTAT3) |
|---|---|---|
| Compound | antagonist IC₅₀ (nM) | antagonist % inhibition |
| 3 | 1.7 | 98 |
| 4 | 6.5 | 100 |
| 5 | 2.1 | 95 |
| 6 | 3.2 | 99 |
| 7 | 0.13 | 98 |
| 8 | 0.053 | 101 |
| 9 | 0.025 | 97 |
| 10 | 0.038 | 99 |
| 11 | 0.68 | 98 |
| 12 | 3.5 | 100 |
| 13 | 6.8 | 99 |
| 14 | 7.2 | 99 |
| 15 | 12 | 97 |
| 16 | 1.4 | 97 |
| 19 | 0.16 | 100 |
| 20 | 0.026 | 101 |
| 21 | 0.063 | 102 |
| 22 | 3.4 | 101 |
| 23 | 5.2 | 99 |
| 24 | 0.058 | 98 |
| 25 | 0.076 | 100 |
| 26 | 0.042 | 100 |
| 27 | 0.23 | 100 |
| 28 | 0.052 | 101 |
| 29 | 0.042 | 99 |
| 30 | 0.077 | 103 |
| 31 | 0.06 | 101 |
| 34 | 0.092 | 102 |
| 36 | 0.034 | 103 |
| 37 | 0.069 | 100 |
| 38 | 1.0 | 100 |
| 39 | 0.51 | 101 |
| 43 | 0.54 | 98 |
| 47 | 0.33 | 98 |
| 49 | 0.093 | 100 |
| 51 | 0.38 | 96 |
| 52 | 0.60 | 99 |
| Ref 4 | 850 | 87 |
| Ref 17 | 370 | — |
| Ref 18 | 18 | 97 |
| Ref 37 | 45 | 89 |
| Ref 41 | 29 | 99 |
| Ref 52 | 7.8 | 103 |
| Ref 78 | 0.13 | 100 |
| Ref 80 | 11 | 97 |
| Ref 81 | 21 | 95 |
| Ref 82 | 11 | 97 |
| Ref 120 | 0.054 | 101 |
| Ref 122 | 0.028 | 99 |
| Ref 125 | 21 | 98 |
| Ref 146 | 42 | 98 |
| Ref 155 | 0.031 | 100 |
| I1 (isomer 3) | Tested | 83 |
| I3 (isomer 3) | 410 | 100 |
| I4 (isomer 3) | Tested | 23 |
| I5 (isomer 3) | 930 | 91 |
| Compound C | 13 | 99 |
| Peptide 993 | 1.4 | 98 |

Tested = IC₅₀ estimated higher than 1 μM when tested in assays

### Examples 3-3 and 3-4: Determination of Peptide Stability in Simulated Gastric Fluid (SIF) and Simulated Intestinal Fluid (SIF)

SGF and SIF were prepared based on the United States Pharmacopeia specifications (Test Solutions, United States Pharmacopeia 35, NF 30, 2012). SGF was prepared by dissolving sodium chloride (0.2 g) in 50 mL of water. A volume of 0.7 mL of 10 M HCl was added to adjust the pH of the solution to 1.2 and the volume made up to 100 mL with water. 64 mg porcine pepsin (P7125, Sigma Aldrich) was gently dissolved into 20 mL preheated (37° C.) solution (3.2 g/L) immediately prior to the incubation. SIF was prepared by dissolving monobasic potassium phosphate (0.68 g) in 50 mL of water and adjusting the pH to 6.8 with 1 M NaOH. The volume was subsequently made up to 100 mL with water. 200 mg porcine pancreatin (P1625, Sigma Aldrich) was gently dissolved into 20 mL preheated (37° C.) solution (10 g/L) immediately prior to the incubation.

To initiate the incubations, 20 μL of peptide stock solution in 50% v/v isopropanol was deposited at the bottom of a well plate and 580 μL matrix solution was added to give a final substrate concentration of 10 μM. The incubation was performed at 37° C. with gentle shaking. 70 μL aliquots were removed at 0, 1 and 4 hours and quenched into 210 μL ice-cold precipitant solution (95% v/v acetonitrile with 0.1% v/v formic acid). After the last timepoint, the sampling plate was mixed for 10 min on a shaking table and centrifuged 10 min at 2200 g. 70 μL resulting supernatant was diluted with 150 μL water, mixed and centrifugated before analysis by liquid chromatography high resolution mass spectrometry. The zero sample was reinjected after the 4-hour sample to confirm that no drift in instrument sensitivity had occurred during the run. Percent remaining at each timepoint was calculated relative to timepoint zero based on absolute peak areas.

The in vitro SIF results (expressed as % peptide remaining after the specified time period) are summarized in Table 3-3 below. Compounds with a SIF stability (after 4 h) above 70% are considered highly SIF stable. Compounds with a SIF stability (after 4 h) between 30-70% (including 30% and 70%) are considered moderately SIF stable. Compounds with a SIF stability (after 4 h) below 30% are considered SIF unstable. SIF stability values above 100% are due to assay uncertainty and indicate the compound has not degraded. Preferred compounds are compounds that exhibit minimal to no degradation, that is have high (at least 70%) SIF stability, preferably as close to 100% SGF stability.

TABLE 3-3
SIF data

| Compound | SIF (% remaining 1 h) | SIF (% remaining 4 h) |
|---|---|---|
| 3 | 115 | 109 |
| 4 | 96 | 89 |
| 5 | 91 | 41 |
| 6 | 97 | 73 |
| 7 | 103 | 130 |
| 8 | 96 | 105 |
| 9 | 102 | 105 |
| 10 | 34 | 11 |
| 11 | 101 | 102 |
| 12 | 60 | 2 |
| 13 | 84 | 56 |
| 14 | 98 | 75 |
| 20 | 109 | 90 |
| 21 | 53 | 57 |
| 24 | 91 | 89 |
| 25 | 88 | 91 |
| 26 | 89 | 91 |
| 27 | 90 | 85 |
| 28 | 106 | 110 |
| 29 | 91 | 92 |
| 30 | 94 | 94 |
| 31 | 77 | 41 |
| 32 | 94 | 65 |
| 33 | 92 | 69 |
| 34 | 87 | 56 |
| 35 | 110 | 96 |
| 36 | 99 | 55 |
| 37 | 78 | 69 |
| 38 | 82 | 65 |
| 39 | 100 | 85 |
| 40 | 93 | 80 |
| 41 | 100 | 95 |
| 43 | 96 | 92 |
| 44 | 78 | 91 |
| 47 | 97 | 92 |
| 49 | 93 | 89 |
| 52 | 92 | 95 |
| Ref 4 | 0 | 0 |
| Ref 17 | 0 | 0 |
| Ref 37 | 0 | 0 |
| Ref 41 | 83 | 79 |
| Ref 78 | 5 | 0 |
| Ref 80 | 84 | 86 |
| Ref 81 | 99 | 77 |
| Ref 82 | 95 | 77 |
| Ref 120 | 82 | 39 |
| Ref 122 | 73 | 25 |
| I1 (isomer 3) | 0 | 0 |
| I3 (isomer 3) | 0 | 0 |
| I4 (isomer 3) | 0 | 0 |
| I5 (isomer 3) | 78 | 22 |
| Compound C | 92 | 76 |
| Peptide 993 | 47 | 22 |

The in vitro SGF results (expressed as % peptide remaining after the specified time period) are summarized in Table 3-4 below. Compounds with a SGF stability (after 4 h) above 70% are considered highly SGF stable. Compounds with a SGF stability (after 4 h) between 30-70% (including 30% and 70%) are considered moderately SGF stable. Compounds with a SGF stability (after 4 h) below 30% are considered SGF unstable. SGF stability values above 100% are due to assay uncertainty and indicate the compound has not degraded. Preferred compounds are compounds that exhibit minimal to no degradation, that is have high (at least 70%) SGF stability, preferably as close to 100% SGF stability.

TABLE 3-4
SGF data

| Compound | SGF (% remaining 1 h) | SGF (% remaining 4 h) |
|---|---|---|
| 3 | 99 | 104 |
| 4 | 97 | 103 |
| 5 | 99 | 105 |
| 6 | 110 | 108 |
| 7 | 77 | 82 |
| 8 | 91 | 87 |
| 9 | 96 | 95 |
| 10 | 97 | 86 |
| 11 | 98 | 97 |
| 12 | 94 | 93 |
| 13 | 94 | 78 |
| 14 | 93 | 82 |
| 20 | 107 | 103 |
| 21 | 101 | 101 |
| 24 | 93 | 90 |
| 25 | 93 | 88 |
| 26 | 105 | 99 |
| 27 | 101 | 96 |
| 28 | 100 | 110 |
| 29 | 94 | 90 |
| 30 | 92 | 89 |
| 31 | 101 | 99 |
| 32 | 96 | 90 |
| 33 | 98 | 93 |
| 34 | 96 | 95 |
| 35 | 101 | 94 |
| 36 | 96 | 103 |
| 37 | 97 | 102 |
| 38 | 100 | 91 |
| 39 | 99 | 96 |
| 40 | 89 | 89 |
| 41 | 101 | 102 |
| 43 | 105 | 98 |
| 47 | 103 | 85 |
| Ref 4 | 1 | 0 |
| Ref 17 | 0 | 0 |
| Ref 37 | 2 | 1 |
| Ref 41 | 93 | 87 |
| Ref 120 | 84 | 81 |
| Ref 122 | 92 | 90 |
| I1 (isomer 3) | 92 | 94 |
| I3 (isomer 3) | 5 | 0 |
| I4 (isomer 3) | 99 | 99 |
| I5 (isomer 3) | 81 | 54 |
| Compound C | 100 | 100 |

### Example 3-5—Functional Inhibition of IL-23 Mediated IFN-γ Secretion by IL-23R Inhibitor Compounds

An in vitro human whole blood assay was used to assess the ability of IL23-R inhibitor compounds to inhibit IL-23-mediated IFN-γ secretion. The assay quantifies the functional antagonism of the IL23R inhibitors may be used to rank inhibitor potencies based on concentration response curves (CRCs) generated in the presence of fixed IL-23, IL-2 and IL-18 concentrations. 1050 and maximal inhibitory response may be calculated from the curves.

The assay procedure was as follows.

Human blood was obtained from voluntary donors at Blodbanken Hvidovre Hospital, Denmark on the day of the experiment by collection in BD Sodium-Heparin Vacutainer tubes [Cat #367876, Avantor].

The buffer used consisted of RPMI-1640 [Cat #61870-010; Thermo Fisher Scientific] supplemented with 1% V/V Penicillin-Streptomycin (Pen-Strep) Solution [Cat #15140; Thermo Fisher Scientific] and 1 mM Sodium Pyruvate [Cat #11360-039, Thermo Fisher Scientific] and 25 mM HEPES buffer [Cat #15630-056, Thermo Fisher Scientific] and 0.05% V/V Casein [Cat #C4765, Sigma-Aldrich] (hereafter referred to as assay buffer). On the day of assay, compounds to be tested for inhibition of hIL-23 mediated signaling were serially diluted in Assay buffer to 4× the final concentration. A stimulatory solution of hIL-23 [Cat #11349-IL; R&D systems] corresponding to 4× the EC80 of hIL-23 (1.6 ng/ml) together with 4× hIL-2 [Cat #BT-002; R&D systems] (40 ng/ml) and 4× hIL-18 [Cat #9124-IL; R&D systems] (80 ng/ml) were also prepared in Assay buffer. To initiate the assay, 25 μL of the prepared 4× diluted test compound was added to the wells of a 96 well plate [Cat #167425; Thermo Scientific] followed by addition of 50 μL human whole blood to each well. Following the preincubation of the blood and inhibitor for 15 min in a cell incubator (37° C., 5% CO2), 25 μL of the prepared 4× stimulatory solution was added to each well and incubated for 16-24 hours in a humidity chamber in a cell incubator (37° C., 5% CO2).

To terminate the assay, 100 μL PBS pH 7.4 [Cat #10010-015; Thermo Fisher Scientific] was added to each well and the plate was spun at 350×G for ten minutes to pellet the blood. The supernatant was removed and added to a 96 well V-bottom Polypropylene plate [Corning 3363] using an 8-channel manual pipette.

Samples were diluted an additional 30× in Diluent 57 buffer from the MSD U-PLEX human IFN-γ assay [Cat #K151TTK-2; Mesoscale Discovery].

The IFN-γ level in the supernatant from the individual wells was determined using the MSD U-PLEX human IFN-γ assay kit [Cat #K151TTK-2; Mesoscale Discovery] and read on a Meso QuickPlex SQ 120 plate reader (Mesoscale Discovery).

For data analysis, raw data counts from the Meso QuickPlex SQ 120 plate reader were converted to concentrations of IFN-γ by using the standard curved supplied with the kit. Compound potency (IC₅₀) and maximal inhibition (% inhibition) was estimated by computer-aided curve fitting using a 4-parameter logistic (4PL) non-linear mode. The IC50 value for a test compound is the concentration at which it achieves 50% of its maximal inhibition of hIL-23 agonist induced response (under a specific condition—here in presence of hIL-2 and hIL-18 as indicated above). Maximal inhibition was expressed as a percentage where 0% is the activity level induced by the reference agonist alone (hIL23R together with IL-2 and IL-18 as indicated above), and 100% is the baseline activity level of the unstimulated receptor in the absence of the reference agonist.

Data for the compound potency (IC₅₀) and maximal inhibition (%) are shown in Table 3-5.

Typically compounds with a low IC₅₀ are desired. Typically compounds capable of completely inhibiting the IL23-induced response are desired.

TABLE 3-5
compound potency (IC₅₀) and maximal inhibition (%)

| Compound no | IC50 (nM) | maximal inhibition (%) |
|---|---|---|
| 20 | 0.64 | 97 |
| 29 | 0.54 | 96 |
| 9 | 0.21 | 97 |
| 43 | 3.9 | 102 |
| 8 | 0.33 | 96 |

### REFERENCES

Cheng and Prusoff, Biochem. Pharmacol., 1973, 22(23), 3099-3108.
Kong et al., Nature Biomedical Engineering, 2020, 4, 560-571.
Sgorbati et al., J. Org. Chem, 2021, 86, 9225-9232.
WO2023/099669
Sayago et al., ACS Med. Chem. Lett., 2018, 9, 912-916.
WO 2016/011208
WO 2017/011820
WO 2018/022937
WO 2018/089693
WO 2018/136646
WO 2020/014646
WO 2021/007433
WO 2021/146441
WO 2021/146458
WO 2023/288017
WO 2023/288019
WO 2023/288028
US 2013/0029907

### CLAUSES

1. A compound of the formula:

Z—R²
wherein
R² is NHR³ wherein R³ is hydrogen or C₁₋₄ alkyl optionally substituted with a pyridyl ring; and
Z is an amino acid sequence of formula I:

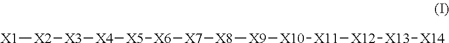

wherein
X1 is absent or is selected from 3-(3-Pyridyl)-Ala or 3-aminopropanoyl;
X2 is selected from the group consisting of 4-aminomethyl-phenylacetyl, 3-(3-Pyridyl)-Ala, and 3-aminopropanoyl;
X3 is selected from the group consisting of Ser, Ala, Phe, and N-Me-Ser;
X4 is Glu or Cys;
X5 is Trp;
X6 is selected from the group consisting of Gln, Q(Me), Q(2Me), Q(pyrrolidin), Ala, Phe, Lys(Ac), Dab(Ac), Cit, and Orn;
X7 is Dab or Cys;
X8 is selected from the group consisting of Y(2-aminoethoxy), homo-Phe, 7-AzaTrp, beta-homo-Trp, and 7-F-Trp;
X9 is selected from the group consisting of 2-Nal, 7-AzaTrp, beta-homo-Trp, and Cyclopropyl-Ala;
X10 is 2-Me-Leu or Aib;
X11 is Glu;
X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser;
X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent; and
X14 is absent or is selected from the group consisting of Sar, D-His, beta-homo-Leu, and 3-(3-Pyridyl)-Ala;
wherein
(i) the Glu at X11 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof;
wherein the compound is not:

| | |
|---|---|
| Ref 80 | [4-Aminomethyl-phenylacetyl](1c)*SC(2a)WQC(2a)[Y(2-aminoethoxy)][2- |
| | Nal][2-Me-Leu]E(1c) [{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid
(1c) denotes the [2,11] lactam bridge; (2a) denotes the [4,7] 1,3-dithio-propan-2-one bridge; (2c) denotes the [4,7] lactam bridge.

2. The compound according to clause 1, wherein the compound is not when X1 is absent; X2 is 4-aminomethyl-phenylacetyl; X3 is Ser or N-Me-Ser; X6 is Gln or Q(pyrrolidin); X8 is Y(2-aminoethoxy); X9 is 2-Nal, X10 is 2-Me-Leu; X12 is Dab, D-Arg, or Ser; X13 is 3-(3-Pyridyl)-Ala or absent; X14 is absent; and R² is NH₂.

3. The compound according to clause 1 or 2, wherein X1 is absent.

4. The compound according to any one of the preceding clauses, wherein X2 is 4-aminomethyl-phenylacetyl.

5. The compound according to any one of the preceding clauses, wherein X3 is Ser or Ala.

6. The compound according to clause 5, wherein X3 is Ser.

7. The compound according to any one of the preceding clauses, wherein X4 is Glu, X7 is Dab, and wherein X4 and X7 together form a lactam bridge.

8. The compound according to any one of the preceding clauses, wherein X6 is Gln.

9. The compound according to any one of the preceding clauses, wherein X8 is Y(2-aminoethoxy).

10. The compound according to any one of the preceding clauses, wherein X9 is 2-Nal.

11. The compound according to any one of the preceding clauses, wherein X10 is 2-Me-Leu.

12. The compound according to any one of the preceding clauses, wherein X12 is Dab.

13. The compound according to any one of the preceding clauses, wherein X13 is 3-(3-Pyridyl)-Ala or is absent.

14. The compound according to clause 13, wherein X13 is 3-(3-Pyridyl)-Ala.

15. The compound according to any one of the preceding clauses, wherein X14 is absent.

16. The compound according to any one of the preceding clauses, wherein R² is NHR³ wherein R³ is hydrogen or C₁₋₄ alkyl.

17. The compound according to clause 16, wherein R² is NH₂.

18. The compound according to clause 16, wherein R² is NHMe.

19. The compound according to any one of clauses 1-6 or 8-18, wherein X4 and X7 are both Cys, and X4 and X7 together form a dithioether bridge, wherein the dithioether bridge between X4 and X7 is of the formula —S-L-Y-L-S—, wherein:
each S is a sulfur atom and is part of the amino acid residue at X4 and X7;
each L is independently C₁₋₄ alkylene; and
Y is either absent or C(═O).

20. The compound according to clause 19, wherein each L is independently C₁₋₂ alkylene.

21. The compound according to clause 20, wherein each L is methylene.

22. The compound according to any one of clauses 19 to 21, wherein Y is C(═O).

23. The compound according to any one of clauses 19 to 22, wherein the dithioether bridge between X4 and X7 is of the formula —SCH₂C(═O)CH₂S—, wherein each S is a sulfur atom and is part of the amino acid residue at X4 and X7.

24. The compound according to any one of the preceding clauses, wherein X1 is absent and X2 is 4-aminomethyl-phenylacetyl.

25. The compound according to any one of the preceding clauses, wherein X1 is absent; X2 is 4-aminomethyl-phenylacetyl; X8 is Y(2-aminoethoxy); X9 is 2-Nal; and X10 is 2-Me-Leu.

26. The compound according to any one of the preceding clauses, wherein X1 is absent; X2 is 4-aminomethyl-phenylacetyl; X3 is Ser or Ala; X8 is Y(2-aminoethoxy); X9 is 2-Nal; and X10 is 2-Me-Leu.

27. The compound according to any one of the preceding clauses, wherein X1 is absent; X2 is 4-aminomethyl-phenylacetyl; X3 is Ser or Ala; X6 is Gln; X8 is Y(2-aminoethoxy); X9 is 2-Nal; and X10 is 2-Me-Leu.

28. The compound according to any one of the preceding clauses, wherein X13 is 3-(3-Pyridyl)-Ala and X14 is absent.

29. The compound according to any one of the preceding clauses, wherein X13 is 3-(3-Pyridyl)-Ala, X14 is absent, and R² is NHMe.

30. The compound according to clause 1 wherein Z is an amino acid sequence selected from the group consisting of a sequence listed in Table 1-1a.

31. A compound according to clause 1 which is selected from a compound from Table 1-1, or a pharmaceutically acceptable salt or solvate thereof.

32. A pharmaceutical composition comprising a compound according to any one of the preceding clauses in combination with a pharmaceutically acceptable carrier, excipient or vehicle.

33. A method for the synthesis of a compound according to any one of clauses 1 to 31, comprising synthesising the analogue by solid-phase or liquid-phase peptide synthesis methodology, optionally isolating and/or purifying the final product, and optionally further comprising the step of forming an amide bond between the amino acid residues at position X1 or at position X2 when X1 is absent and at position X11, and optionally further comprising the step of forming an amide bond or forming two thioether bonds with a linker between the amino acid residues at positions X4 and X7.

34. A compound according to any one of clauses 1 to 31, or a pharmaceutical composition according to clause 32, for use in a method of medical treatment.

35. A compound according to any one of clauses 1 to 31, or a pharmaceutical composition according to clause 32, for use in a method of prevention or treatment of inflammatory bowel disease (IBD) such as Crohn's Disease or ulcerative colitis, psoriasis, psoriatic arthritis, and combinations thereof.

36. The compound or pharmaceutical composition for use according to clause 35, wherein the compound or pharmaceutical composition is for use in a method of prevention or treatment of inflammatory bowel disease (IBD) and/or psoriasis.

37. Use of a compound according to any one of clauses 1 to 31, or a pharmaceutical composition according to clause 32, in the manufacture of a medicament for the prevention or treatment of inflammatory bowel disease (IBD) such as Crohn's Disease or ulcerative colitis, psoriasis, psoriatic arthritis, and combinations thereof.

38. The use of the compound or pharmaceutical composition according to clause 37, wherein the use of the compound or pharmaceutical compositions is in the manufacture of a medicament for the prevention or treatment of inflammatory bowel disease (IBD) and/or psoriasis.

39. A method of prevention or treatment of inflammatory bowel disease (IBD) such as Crohn's Disease or ulcerative colitis, psoriasis, psoriatic arthritis, and combinations thereof; which comprises administering to a subject an effective amount of a compound according to any one of clauses 1 to 31, or a pharmaceutical composition according to clause 32.

40. The method of prevention or treatment according to clause 39, wherein the method of prevention or treatment is for inflammatory bowel disease (IBD) and/or psoriasis.

41. The pharmaceutical composition according to clause 32, wherein said pharmaceutical composition is for oral administration.

1. A compound of the formula: 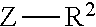
wherein
R² is NHR³ or C(═O)R³, wherein R³ is hydrogen or C₁₋₄ alkyl optionally substituted with a pyridyl ring; and
Z is an amino acid sequence of formula I: 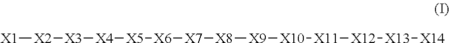
wherein
X1 is absent or is selected from 3-(3-Pyridyl)-Ala or 3-aminopropanoyl;
X2 is selected from the group consisting of 4-aminomethyl-phenylacetyl, an alanine residue substituted by a carbocyclic group or an aromatic or heteroaromatic group selected from the group consisting of phenyl, pyridyl, naphthyl and quinolinyl, each optionally substituted, 3-aminopropanoyl, Lys, D-Lys, iso-Lys, D-iso-Lys, beta-Lys, D-beta-Lys, homo-Lys, D-homo-Lys, beta-homo-Lys, N-Me-Lys, N-Me-homo-Lys,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X3 is selected from any amino acid or ω-hydroxy-C₂₋₆ alkanoic acid;
X4 is selected from Glu, D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-Lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys and N-Me-homo-Cys;
X5 is selected from the group consisting of an optionally substituted tryptophan residue, an optionally substituted azatryptophan residue, and an optionally substituted beta-homotryptophan residue;
X6 is selected from the group consisting of an optionally substituted Gln residue, an optionally substituted Lys residue, an optionally substituted Arg residue, an optionally substituted Dab residue, an optionally substituted Orn residue, an optionally substituted Phe residue, Ala, D-Ala, beta-Ala, D-beta-Ala, homo-Ala, D-homo-Ala, beta-homo-Ala, N-Me-Ala, N-Me-homo-Ala, Cit, D-Cit, beta-Cit, D-beta-Cit, homo-Cit, D-homo-Cit, beta-homo-Cit, N-Me-Cit, N-Me-homo-Cit, Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Tyr, D-Tyr, beta-Tyr, D-beta-Tyr, homo-Tyr, D-homo-Tyr, beta-homo-Tyr, N-Me-Tyr, N-Me-homo-Tyr, Val, D-Val, beta-Val, D-beta-Val, homo-Val, D-homo-Val, beta-homo-Val, N-Me-Val, N-Me-homo-Val or His, D-His, beta-His, D-beta-His, homo-His, D-homo-His, beta-homo-His, N-Me-His and N-Me-homo-His;
X7 is selected from Glu, D-Glu, iso-Glu, D-iso-Glu, beta-Glu, D-beta-Glu, homo-Glu, D-homo-Glu, beta-homo-Glu, N-Me-Glu, N-Me-homo-Glu,
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Lys, D-Lys, iso-Lys, D-iso-Lys, beta-Lys, D-beta-Lys, homo-Lys, D-homo-Lys, beta-homo-Lys, N-Me-Lys, N-Me-homo-Lys,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Cys, D-Cys, beta-Cys, D-beta-Cys, homo-Cys, D-homo-Cys, beta-homo-Cys, N-Me-Cys, and N-Me-homo-Cys;
X8 is selected from the group consisting of an optionally substituted tryptophan residue, an optionally substituted azatryptophan residue, an optionally substituted beta-homotryptophan residue, an optionally substituted tyrosine residue, an optionally substituted phenylalanine residue, an optionally substituted homophenylalanine residue, and an alanine residue substituted by an optionally substituted carbocylic group or an aromatic group selected from the group consisting of phenyl, pyridyl, naphthyl and quinolinyl, each optionally substituted;
X9 is selected from the group consisting of an optionally substituted tryptophan residue, an optionally substituted azatryptophan residue, an optionally substituted beta-homotryptophan residue, an optionally substituted alanine residue, an optionally substituted phenylalanine and an optionally substituted tyrosine residue;
X10 is selected from the group consisting of Val, D-Val, beta-Val, D-beta-Val, homo-Val, D-homo-Val, beta-homo-Val, N-Me-Val, N-Me-homo-Val, 2-Me-Val,
Gly, beta-Gly, homo-Gly, beta-homo-Gly, N-Me-Gly, N-Me-homo-Gly,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Lys, D-Lys, iso-Lys, D-iso-Lys, beta-Lys, D-beta-Lys, homo-Lys, D-homo-Lys, beta-homo-Lys, N-Me-Lys, N-Me-homo-Lys, 2-Me-Lys,
Aib, D-Aib, beta-Aib, D-beta-Aib, homo-Aib, D-homo-Aib, beta-homo-Aib, N-Me-Aib, N-Me-homo-Aib,
Ala, D-Ala, beta-Ala, D-beta-Ala, homo-Ala, D-homo-Ala, beta-homo-Ala, N-Me-Ala, N-Me-homo-Ala,
Leu, D-Leu, beta-Leu, D-beta-Leu, homo-Leu, D-homo-Leu, beta-homo-Leu, N-Me-Leu, N-Me-homo-Leu, 2-Me-Leu,
Ile, D-Ile, beta-Ile, D-beta-Ile, homo-Ile, D-homo-Ile, beta-homo-Ile, N-Me-Ile, N-Me-homo-Ile,
and a carbocyclic or heterocyclic ring having an amino substituent and a carbonyl substituent;
X11 is selected from the group consisting of
Dpr, D-Dpr, iso-Dpr, D-iso-Dpr, beta-Dpr, D-beta-Dpr, homo-Dpr, D-homo-Dpr, beta-homo-Dpr, N-Me-Dpr, N-Me-homo-Dpr,
Dab, D-Dab, iso-Dab, D-iso-Dab, beta-Dab, D-beta-Dab, homo-Dab, D-homo-Dab, beta-homo-Dab, N-Me-Dab, N-Me-homo-Dab,
Orn, D-Orn, iso-Orn, D-iso-Orn, beta-Orn, D-beta-Orn, homo-Orn, D-homo-Orn, beta-homo-Orn, N-Me-Orn, N-Me-homo-Orn,
Lys, D-Lys, iso-Lys, D-iso-Lys, beta-Lys, D-beta-Lys, homo-Lys, D-homo-Lys, beta-homo-Lys, N-Me-Lys, N-Me-homo-Lys, Lys(Me),
Lys(Gly),
Asp, D-Asp, iso-Asp, D-iso-Asp, beta-Asp, D-beta-Asp, homo-Asp, D-homo-Asp, beta-homo-Asp, N-Me-Asp, N-Me-homo-Asp,
Glu, D-Glu, iso-Glu, D-iso-Glu, beta-Glu, D-beta-Glu, homo-Glu, D-homo-Glu, beta-homo-Glu, N-Me-Glu, N-Me-homo-Glu, and
2-amino-6-carboxyhexanoyl;
X12 is selected from the group consisting of an optionally substituted Phe residue, an optionally substituted Tyr residue, an optionally substituted His residue, an alanine residue substituted by a carbocyclic group or an aromatic or heteroaromatic group selected from the group consisting of phenyl, pyridyl, naphthyl and quinolinyl, each optionally substituted, Dab, D-Dab, iso-Dab, D-iso-Dab, beta-Dab, D-beta-Dab, homo-Dab, D-homo-Dab, beta-homo-Dab, N-Me-Dab, N-Me-homo-Dab, Gly, beta-Gly, homo-Gly, beta-homo-Gly, N-Me-Gly, N-Me-homo-Gly, Pro, 5-aminopentanoyl, 4-aminopiperidin-4-carbonyl, (R,S)-imidazolidin-2-carbonyl, 3-aminopropanoyl, Gly-CF3, D-Gly-CF3, Nle, Gln, D-Gln, iso-Gln, D-iso-Gln, beta-Gln, D-beta-Gln, homo-Gln, D-homo-Gln, beta-homo-Gln, N-Me-Gln, N-Me-homo-Gln, THP, Ser, D-Ser, beta-Ser, D-beta-Ser, homo-Ser, D-homo-Ser, beta-homo-Ser, N-Me-Ser, 2-Me-Ser, N-Me-homo-Ser, Ser(OMe), 3-aminotetrahydrofuran-3-carbonyl, THP, Arg, D-Arg, beta-Arg, D-beta-Arg, homo-Arg, D-homo-Arg, beta-homo-Arg, N-Me-Arg, N-Me-homo-Arg, Thr, D-Thr, beta-Thr, D-beta-Thr, homo-Thr, D-homo-Thr, beta-homo-Thr, N-Me-Thr, N-Me-homo-Thr Glu, D-Glu, iso-Glu, D-iso-Glu, beta-Glu, D-beta-Glu, homo-Glu, D-homo-Glu, beta-homo-Glu, N-Me-Glu, N-Me-homo-Glu, Asn, D-Asn, beta-Asn, D-beta-Asn, homo-Asn, D-homo-Asn, beta-homo-Asn, N-Me-Asn, N-Me-homo-Asn, GABA, 2-(trimethyl-2-aminoethoxy)ethoxy]propyl and Lys wherein the side chain —NH₂ of the Lys is substituted with —C(═O)(CH₂)ₙR^{K} wherein n is 0-2 and R^{K} is imidazolyl, pyrimidyl, or pyridyl optionally substituted with F;
X13 is selected from the group consisting of an optionally substituted His residue, an optionally substituted Phe residue, an alanine residue substituted by a carbocyclic group or an aromatic or heteroaromatic group selected from the group consisting of phenyl, pyridyl, naphthyl and quinolinyl, each optionally substituted, Asn, D-Asn, beta-Asn, D-beta-Asn, homo-Asn, D-homo-Asn, beta-homo-Asn, N-Me-Asn, N-Me-homo-Asn, Gly, beta-Gly, homo-Gly, beta-homo-Gly, N-Me-Gly, N-Me-homo-Gly, and Dab, Orn or Lys wherein the side chain —NH₂ is substituted with —C(═O)(CH₂)ₙR^{K} wherein n is 0-2 and R^{K} is imidazolyl, pyrimidyl, or pyridyl optionally substituted with F, or is absent; and
X14 is absent or is selected from the group consisting of Sar, an optionally substituted His residue, Leu, D-Leu, beta-Leu, D-beta-Leu, homo-Leu, D-homo-Leu, beta-homo-Leu, N-Me-Leu, N-Me-homo-Leu, 2-Me-Leu, and an alanine residue substituted by a carbocyclic group or an aromatic or heteroaromatic group selected from the group consisting of phenyl, pyridyl, naphthyl and quinolinyl, each optionally substituted;
wherein
(i) the amino acid residue at X1 forms a lactam bridge with the amino acid residue at X1 or at X2 when X1 is absent; and
(ii) X4 and X7 are amino acid residues who together form a lactam bridge or a dithioether bridge;
or a pharmaceutically acceptable salt or solvate thereof;
wherein the compound is not:

| | |
|---|---|
| Ref 52 | [4-Aminomethyl-phenylacetyl](1c)*SC(2a)WQC(2a)[Y(2-aminoethoxy)][2- |
| | Nal][2-Me-Leu]E(1c)R-[NH2] |
| Ref 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| Ref 81 | [4-Aminomethyl-phenylacetyl](1c)*SC(2a)WQC(2a)[Y(2-aminoethoxy)][2- |
| | Nal][2-Me-Leu]E(1c)S-[NH2] |
| Ref 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| Ref 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| Ref 112 | [Dab](1c)*SC(2a)WQC(2a)[Y(2-aminoethoxy)][2-Nal][2-Me- |
| | Leu]E(1c)[{d}R]-[NH2] |
| Ref 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| Ref 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| Ref 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| Ref 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| Ref 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| Ref 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| Ref 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| Ref 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| Ref 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| Ref 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| Ref 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| Ref 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| Ref 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| Ref 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| Ref 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| Ref 130 | [4-Aminomethyl-phenylacetyl](1c)*SE(2c)WQ[Dab](2c)F[2-Nal][2-Me- |
| | Leu]E(1c)[Dab]-[NH2] |
| Ref 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| Ref 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| Ref 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| Ref 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| Ref 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| Ref 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| Ref 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| Ref 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab]-[NH2] |
| Ref 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab]-[NH2] |
| Ref 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab]-[NH2] |
| Ref 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| Ref 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| Ref 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| Ref 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| Ref 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| Ref 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| Ref 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| Ref 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| Ref 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| Ref 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| Ref 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| Ref 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| Ref 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| Ref 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| Ref 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| Ref 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| Ref 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| Ref 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala]-[NH2] |
| Ref 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| Ref 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| Ref 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| Ref 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| Ref 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
* denotes that the (1c), (2a) and (2c) bridges use the peptide backbone amine or carboxylic acid at the N- or C-terminus, not the side chain amine or carboxylic acid,
(1c) denotes the [2,11] lactam bridge; (2a) denotes the [4,7] 1,3-dithio-propan-2-one bridge; (2c) denotes the [4,7] lactam bridge.

2. The compound of claim 1, wherein X2 is selected from the group consisting of 4-aminomethyl-phenylacetyl, 3-(3-Pyridyl)-Ala, and 3-aminopropanoyl.

3. The compound of any one of claim 1 or 2, wherein X3 is selected from the group consisting of Ser, Ala, N-Me-Ala, Phe, and N-Me-Ser.

4. The compound of any one of claims 1 to 3, wherein X4 is Glu or Cys.

5. The compound of any one of claims 1 to 4, wherein X5 is Trp or 7-Me-Trp.

6. The compound of any one of claims 1 to 5, wherein X6 is selected from the group consisting of Gln, Q(Me), Q(2Me), Q(pyrrolidin), Ala, Phe, Lys(Ac), Lys(NMePEG3), Dab(Ac), Cit, and Orn.

7. The compound of any one of claims 1 to 6, wherein X7 is selected from Dab or Cys.

8. The compound of any one of claims 1 to 7, wherein X8 is selected from the group consisting of Y(2-aminoethoxy), homo-Phe, F(4-morpholine), 3-quinolinylalanine, Y(2-aminoethoxy)(N(Me)2, Y(nPentylamine)(N⁺(Me)3), Y(2-trimethyl-PEG2), Y(2-aminoethoxy)(N(Me)2), 7-AzaTrp, beta-homo-Trp, and 7-F-Trp.

9. The compound of any one of claims 1 to 8, wherein X9 is selected from the group consisting of 2-Nal, 7-AzaTrp, beta-homo-Trp, 3-quinolinylalanine and cyclopropyl-Ala.

10. The compound of any one of claims 1 to 9, wherein X10 is 2-Me-Leu or Aib.

11. The compound of any one of claims 1 to 10, wherein X11 is Glu.

12. The compound of any one of claims 1 to 11, wherein X12 is selected from the group consisting of Dab, iso-Dab, D-Arg, Gly-CF3, D-Gly-CF3, Nle, Gln, His, THP, Ser, D-Ser, 2-Me-Ser, Ser(OMe), and homo-Ser.

13. The compound of any one of claims 1 to 12, wherein X13 is selected from the group consisting of 3-(3-Pyridyl)-Ala, D-3-(3-Pyridyl)-Ala, 2-Me-3-(3-Pyridyl)-Ala, N-Me-3-(3-Pyridyl)-Ala, 3-(3,5-Pyrimidyl)-Ala, His, D-His, and His(Me), or is absent.

14. The compound of any one of claims 1 to 13, wherein X14 is absent or is selected from the group consisting of Sar, D-His, beta-homo-Leu, and 3-(3-Pyridyl)-Ala.

15. The compound according to any one of claims 1 to 14, wherein the compound is not when X1 is absent; X2 is 4-aminomethyl-phenylacetyl; X3 is Ser or N-Me-Ser; X6 is Gln or Q(pyrrolidin); X8 is Y(2-aminoethoxy); X9 is 2-Nal, X10 is 2-Me-Leu; X12 is Dab, D-Arg, or Ser; X13 is 3-(3-Pyridyl)-Ala or absent; X14 is absent; and R² is NH₂.

16. The compound according to any one of claims 1 to 15, wherein X1 is absent;
optionally wherein X2 is 4-aminomethyl-phenylacetyl;
optionally wherein X3 is Ser or Ala, optionally wherein X3 is Ser;
optionally wherein X6 is Gln.

17. The compound according to any one of the preceding claims, wherein X8 is Y(2-aminoethoxy);
optionally wherein X9 is 2-Nal;
optionally wherein X10 is 2-Me-Leu;
optionally wherein X12 is Dab.

18. The compound according to any one of the preceding claims, wherein X13 is 3-(3-Pyridyl)-Ala or is absent; optionally wherein X13 is 3-(3-Pyridyl)-Ala;
optionally wherein X14 is absent.

19. The compound according to any one of the preceding claims, wherein R² is NHR³ wherein R³ is hydrogen or C₁₋₄ alkyl; optionally wherein R² is NH₂ or NHMe.

20. The compound according to any one of the preceding claims, wherein X4 is Glu, X7 is Dab, and wherein X4 and X7 together form a lactam bridge.

21. The compound according to any one of claims 1-19, wherein X4 and X7 are both Cys, and X4 and X7 together form a dithioether bridge, wherein the dithioether bridge between X4 and X7 is of the formula —S-L-Y-L-S—, wherein:
each S is a sulfur atom and is part of the amino acid residue at X4 and X7;
each L is independently C₁₋₄ alkylene; and
Y is either absent or C(═O);
optionally wherein each L is independently C₁₋₂ alkylene, optionally wherein each L is methylene;
optionally wherein Y is C(═O);
optionally wherein the dithioether bridge between X4 and X7 is of the formula —SCH₂C(═O)CH₂S—, wherein each S is a sulfur atom and is part of the amino acid residue at X4 and X7.

22. The compound according to any one of the preceding claims, wherein:
X1 is absent and X2 is 4-aminomethyl-phenylacetyl; or
X1 is absent; X2 is 4-aminomethyl-phenylacetyl; X8 is Y(2-aminoethoxy); X9 is 2-Nal; and
X10 is 2-Me-Leu; or
X1 is absent; X2 is 4-aminomethyl-phenylacetyl; X3 is Ser or Ala; X8 is Y(2-aminoethoxy);
X9 is 2-Nal; and X10 is 2-Me-Leu; or
X1 is absent; X2 is 4-aminomethyl-phenylacetyl; X3 is Ser or Ala; X6 is Gln; X8 is Y(2-aminoethoxy); X9 is 2-Nal; and X10 is 2-Me-Leu; or
X13 is 3-(3-Pyridyl)-Ala and X14 is absent; or
X13 is 3-(3-Pyridyl)-Ala, X14 is absent, and R² is NHMe.

23. The compound according to claim 1 wherein Z is an amino acid sequence selected from the group consisting of:

| SEQ ID NO: | Sequence |
|---|---|
| SEQ ID NO: 1 | [3-Aminopropanoyl](4c)*[3-(3-Pyridyl)-Ala]SC(2a)WQC(2a)[Y(2- |
| | aminoethoxy)][2-Nal][2-Me-Leu]E(4c)[{d}R] |
| SEQ ID NO: 2 | [3-(3-Pyridyl)-Ala](4c)*[3-Aminopropanoyl]SC(2a)WQC(2a)[Y(2- |
| | aminoethoxy)][2-Nal][2-Me-Leu]E(4c)[{d}R] |
| SEQ ID NO: 3 | [4-Aminomethyl-phenylacetyl](1c)*SE(2c)WQ[Dab](2c)[Y(2- |
| | aminoethoxy)][2-Nal][2-Me-Leu]E(1c)[Dab][2-Me-3-(3-Pyridyl)-Ala] |
| SEQ ID NO: 4 | [4-Aminomethyl-phenylacetyl](1c)*SE(2c)WQ[Dab](2c)[Y(2- |
| | aminoethoxy)][2-Nal][2-Me-Leu]E(1c)[Dab][N-Me-3-(3-Pyridyl)-Ala] |
| SEQ ID NO: 5 | [4-Aminomethyl-phenylacetyl](1c)*SE(2c)WQ[Dab](2c)[Y(2- |
| | aminoethoxy)][2-Nal][2-Me-Leu]E(1c)[Dab][{d}3-(3-Pyridyl)-Ala] |
| SEQ ID NO: 6 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 7 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 8 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 9 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][Sar] |
| SEQ ID NO: 10 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][{d}H] |
| SEQ ID NO: 11 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala][beta-hLeu] |
| SEQ ID NO: 12 | [4-Aminomethyl-phenylacetyl](1c)*SE(2c)WQ[Dab](2c)[Y(2- |
| | aminoethoxy)][2-Nal][2-Me-Leu]E(1c)[Dab]H[3-(3-Pyridyl)-Ala] |
| SEQ ID NO: 13 | [4-Aminomethyl-phenylacetyl](1c)*SE(2c)WQ[Dab](2c)[Y(2- |
| | aminoethoxy)][2-Nal][2-Me-Leu]E(1c)[Dab][{d}H] |
| SEQ ID NO: 14 | [4-Aminomethyl-phenylacetyl](1c)*SE(2c)WQ[Dab](2c)[Y(2- |
| | aminoethoxy)][2-Nal][2-Me-Leu]E(1c)[Dab][H(Me)] |
| SEQ ID NO: 15 | [4-Aminomethyl-phenylacetyl](1c)*SE(2c)WQ[Dab](2c)[Y(2- |
| | aminoethoxy)][2-Nal][2-Me-Leu]E(1c)[Iso-Dab][2-Me-3-(3-Pyridyl)-Ala] |
| SEQ ID NO: 16 | [4-Aminomethyl-phenylacetyl](1c)*SE(2c)WQ[Dab](2c)[Y(2- |
| | aminoethoxy)][2-Nal][2-Me-Leu]E(1c)[{d}Gly-CF3][3-(3-Pyridyl)-Ala] |
| SEQ ID NO: 17 | [4-Aminomethyl-phenylacetyl](1c)*SE(2c)WQ[Dab](2c)[Y(2- |
| | aminoethoxy)][2-Nal][2-Me-Leu]E(1c)[Gly-CF3][3-(3-Pyridyl)-Ala] |
| SEQ ID NO: 18 | [4-Aminomethyl-phenylacetyl](1c)*[N-Me-Ser]E(2c)WQ[Dab](2c)[Y(2- |
| | aminoethoxy)][2-Nal][2-Me-Leu]E(1c)[Dab][3-(3-Pyridyl)-Ala] |
| SEQ ID NO: 19 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3,5-Pyrimidyl)-Ala] |
| SEQ ID NO: 20 | [4-Aminomethyl-phenylacetyl](1c)*AE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 21 | [4-Aminomethyl-phenylacetyl](1c)*FE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 22 | [4-Aminomethyl-phenylacetyl](1c)*SE(2c)WA[Dab](2c)[Y(2- |
| | aminoethoxy)][2-Nal][2-Me-Leu]E(1c)[Dab][3-(3-Pyridyl)-Ala] |
| SEQ ID NO: 23 | [4-Aminomethyl-phenylacetyl](1c)*SE(2c)WF[Dab](2c)[Y(2- |
| | aminoethoxy)][2-Nal][2-Me-Leu]E(1c)[Dab][3-(3-Pyridyl)-Ala] |
| SEQ ID NO: 24 | [4-Aminomethyl-phenylacetyl](1c)*SE(2c)W[K(Ac)][Dab](2c)[Y(2- |
| | aminoethoxy)][2-Nal][2-Me-Leu]E(1c)[Dab][3-(3-Pyridyl)-Ala] |
| SEQ ID NO: 25 | [4-Aminomethyl-phenylacetyl](1c)*SE(2c)W[Dab(Ac)][Dab](2c)[Y(2- |
| | aminoethoxy)][2-Nal][2-Me-Leu]E(1c)[Dab][3-(3-Pyridyl)-Ala] |
| SEQ ID NO: 26 | [4-Aminomethyl-phenylacetyl](1c)*SE(2c)W[Cit][Dab](2c)[Y(2- |
| | aminoethoxy)][2-Nal][2-Me-Leu]E(1c)[Dab][3-(3-Pyridyl)-Ala] |
| SEQ ID NO: 27 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab][3-(3-Pyridyl)-Ala] |
| SEQ ID NO: 28 | [4-Aminomethyl-phenylacetyl](1c)*SE(2c)W[Orn][Dab](2c)[Y(2- |
| | aminoethoxy)][2-Nal][2-Me-Leu]E(1c)[Dab][3-(3-Pyridyl)-Ala] |
| SEQ ID NO: 29 | [4-Aminomethyl-phenylacetyl](1c)*SE(2c)W[Q(Me)][Dab](2c)[Y(2- |
| | aminoethoxy)][2-Nal][2-Me-Leu]E(1c)[Dab][3-(3-Pyridyl)-Ala] |
| SEQ ID NO: 30 | [4-Aminomethyl-phenylacetyl](1c)*SE(2c)W[Q(2Me)][Dab](2c)[Y(2- |
| | aminoethoxy)][2-Nal][2-Me-Leu]E(1c)[Dab][3-(3-Pyridyl)-Ala] |
| SEQ ID NO: 31 | [4-Aminomethyl-phenylacetyl](1c)*SE(2c)WQ[Dab](2c)[Y(2- |
| | aminoethoxy)][2-Nal][2-Me-Leu]E(1c)S[3-(3-Pyridyl)-Ala] |
| SEQ ID NO: 32 | [4-Aminomethyl-phenylacetyl](1c)*SE(2c)WQ[Dab](2c)[Y(2- |
| | aminoethoxy)][2-Nal][2-Me-Leu]E(1c)[Nle][3-(3-Pyridyl)-Ala] |
| SEQ ID NO: 33 | [4-Aminomethyl-phenylacetyl](1c)*SE(2c)WQ[Dab](2c)[Y(2- |
| | aminoethoxy)][2-Nal][2-Me-Leu]E(1c)Q[3-(3-Pyridyl)-Ala] |
| SEQ ID NO: 34 | [4-Aminomethyl-phenylacetyl](1c)*SE(2c)WQ[Dab](2c)[Y(2- |
| | aminoethoxy)][2-Nal][2-Me-Leu]E(1c)H[3-(3-Pyridyl)-Ala] |
| SEQ ID NO: 35 | [4-Aminomethyl-phenylacetyl](1c)*SE(2c)WQ[Dab](2c)[Y(2- |
| | aminoethoxy)][2-Nal][2-Me-Leu]E(1c)[THP][3-(3-Pyridyl)-Ala] |
| SEQ ID NO: 36 | [4-Aminomethyl-phenylacetyl](1c)*SE(2c)WQ[Dab](2c)[Y(2- |
| | aminoethoxy)][2-Nal][2-Me-Leu]E(1c)[hSer][3-(3-Pyridyl)-Ala] |
| SEQ ID NO: 37 | [4-Aminomethyl-phenylacetyl](1c)*SE(2c)WQ[Dab](2c)[Y(2- |
| | aminoethoxy)][2-Nal][2-Me-Leu]E(1c)[Ser(OMe)][3-(3-Pyridyl)-Ala] |
| SEQ ID NO: 38 | [4-Aminomethyl-phenylacetyl](1c)*SE(2c)WQ[Dab](2c)[Y(2- |
| | aminoethoxy)][2-Nal][2-Me-Leu]E(1c)[{d}S][3-(3-Pyridyl)-Ala] |
| SEQ ID NO: 39 | [4-Aminomethyl-phenylacetyl](1c)*SE(2c)WQ[Dab](2c)[Y(2- |
| | aminoethoxy)][2-Nal][2-Me-Leu]E(1c)[2-Me-Ser][3-(3-Pyridyl)-Ala] |
| SEQ ID NO: 40 | [4-Aminomethyl-phenylacetyl](1c)*AE(2c)WQ[Dab](2c)[Y(2- |
| | aminoethoxy)][2-Nal][2-Me-Leu]E(1c)S[3-(3-Pyridyl)-Ala] |
| SEQ ID NO: 41 | [4-Aminomethyl-phenylacetyl](1c)*AE(2c)WQ[Dab](2c)[hPhe][2- |
| | Nal][Aib]E(1c)[Dab][3-(3-Pyridyl)-Ala] |
| SEQ ID NO: 42 | [4-Aminomethyl-phenylacetyl](1c)*AE(2c)WQ[Dab](2c)[Y(2- |
| | aminoethoxy)][Cyclopropyl-Ala][Aib]E(1c)[Dab][3-(3-Pyridyl)-Ala] |
| SEQ ID NO: 43 | [4-Aminomethyl-phenylacetyl](1c)*AE(2c)WQ[Dab](2c)[7-AzaTrp][2- |
| | Nal][Aib]E(1c)[Dab][3-(3-Pyridyl)-Ala] |
| SEQ ID NO: 44 | [4-Aminomethyl-phenylacetyl](1c)*AE(2c)WQ[Dab](2c)[Y(2- |
| | aminoethoxy)][7-AzaTrp][Aib]E(1c)[Dab][3-(3-Pyridyl)-Ala] |
| SEQ ID NO: 45 | [4-Aminomethyl-phenylacetyl](1c)*AE(2c)WQ[Dab](2c)[beta-h Trp][2- |
| | Nal][Aib]E(1c)[Dab][3-(3-Pyridyl)-Ala] |
| SEQ ID NO: 46 | [4-Aminomethyl-phenylacetyl](1c)*AE(2c)WQ[Dab](2c)[Y(2- |
| | aminoethoxy)][beta-hTrp][Aib]E(1c)[Dab][3-(3-Pyridyl)-Ala] |
| SEQ ID NO: 47 | [4-Aminomethyl-phenylacetyl](1c)*AE(2c)WQ[Dab](2c)[7-F-Trp][2- |
| | Nal][Aib]E(1c)[Dab][3-(3-Pyridyl)-Ala] |
| SEQ ID NO: 48 | [4-aminomethyl-phenylacetyl(1c)*][N-Me-Ala]E(2c)WQ[Dab](2c)[Y(2- |
| | aminoethoxy)][2Nal][Aib]E(1c)[Dab][3-(3-Pyridyl)-alanyl] |
| SEQ ID NO: 49 | 4-aminomethyl-phenylacetyl](1c)*AE(2c)WQ[Dab](2c)[Y(2- |
| | aminoethoxy)][3-quinolinylalanine][Aib]E(1c)[Dab][3-(3-Pyridyl)-alanyl] |
| SEQ ID NO: 51 | [4-aminomethyl-phenylacetyl](1c)*AE(2c)WQ[Dab](2c)[F(4- |
| | morpholine)][2Nal][Aib]E(1c)[Dab][3-(3-Pyridyl)-alanyl] |
| SEQ ID NO: 52 | [4-aminomethyl-phenylacetyl](1c)*AE(2c)WQ[Dab](2c)[3- |
| | quinolinylalanine][2Nal][Aib]E(1c)[Dab][3-(3-Pyridyl)-alanyl] |
| SEQ ID NO: 53 | [4-aminomethyl-phenylacetyl](1c)*AE(2c)WQ[Dab](2c)[F(4- |
| | morpholine)][2Nal][Aib]E(1c)[Ser(OCH3)][3-(3-Pyridyl)-alanyl] |
| SEQ ID NO: 54 | [4-aminomethyl-phenylacetyl](1c)*[3-Aminopropanoyl]E(2c)[7-Me- |
| | Trp][Q(2Me)][Dab](2c)[Y(2-aminoethoxy)(N(Me)2)][2Nal][Aib]E(1c)[Dab][3- |
| | (3-Pyridyl)-alanyl] |
| SEQ ID NO: 55 | [4-aminomethyl-phenylacetyl](1c)*[3-Aminopropanoyl]E(2c)[7-Me- |
| | Trp][Q(2Me)][Dab](2c)[Y(nPentylamine)(N+(Me)3)][2Nal][Aib]E(1c)[Dab][3- |
| | (3-Pyridyl)-alanyl] |
| SEQ ID NO: 56 | [4-aminomethyl-phenylacetyl](1c)*[3-Aminopropanoyl]E(2c)[7-Me- |
| | Trp][Q(2Me)][Dab](2c)[Tyr(2-trimethyl-PEG3)][2Nal][Aib]E(1c)[Dab][3-(3- |
| | Pyridyl)-alanyl] |
| SEQ ID NO: 57 | [4-aminomethyl-phenylacetyl](1c)*[3-Aminopropanoyl]E(2c)[7-Me- |
| | Trp][K(NMePEG3)][Dab](2c)[Y(2- |
| | aminoethoxy)(N(Me)2)][2Nal][Aib]E(1c)[Dab][3-(3-Pyridyl)-alanyl] |

wherein:
* denotes that the (1c), (2a), (2c), and (4c) bridges use the peptide backbone amine or carboxylic acid at the N- or C-terminus, not the side chain amine or carboxylic acid,
(1c) denotes the [2,11] lactam bridge; (2a) denotes the [4,7] 1,3-dithio-propan-2-one bridge; (2c) denotes the [4,7] lactam bridge; (4c) denotes the [1,11] lactam bridge.

24. A compound according to claim 1 which is selected from:

| Compound | Structure |
|---|---|
| 1 | [3-Aminopropanoyl](4c)*[3-(3-Pyridyl)-Ala]SC(2a)WQC(2a)[Y(2- |
| | aminoethoxy)][2-Nal][2-Me-Leu]E(4c)[{d}R]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CSCC(═O)CSC[C@H](NC(═O)[C@H](CO)NC |
| | (═O)[C@H](Cc3cccnc3)NC(═O)CCNC(═O)CC[C@@H](C(═O)N[C@H](CCCNC |
| | (═N)N)C(N)═O)NC1═O)C(═O)N[C@@H](Cc1c[nH]c3ccccc13)C(═O)N[C@@H] |
| | (CCC(N)═O)C(═O)N2 |
| 2 | [3-(3-Pyridyl)-Ala](4c)*[3-Aminopropanoyl]SC(2a)WQC(2a)[Y(2- |
| | aminoethoxy)][2-Nal][2-Me-Leu]E(4c)[{d}R]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CSCC(═O)CSC[C@H](NC(═O)[C@H](CO)NC |
| | (═O)CCNC(═O)[C@H](Cc3cccnc3)NC(═O)CC[C@@H](C(═O)N[C@H](CCCNC |
| | (═N)N)C(N)═O)NC1═O)C(═O)N[C@@H](Cc1c[nH]c3ccccc13)C(═O)N[C@@H] |
| | (CCC(N)═O)C(═O)N2 |
| 3 | [4-Aminomethyl-phenylacetyl](1c)*SE(2c)WQ[Dab](2c)[Y(2-aminoethoxy)][2- |
| | Nal][2-Me-Leu]E(1c)[Dab][2-Me-3-(3-Pyridyl)-Ala]-[NH2] |
| | CC(C)C[C@]1(C)NC(═O)[C@H](Cc2ccc3ccccc3c2)NC(═O)[C@H](Cc2cccCc3ccc |
| | (OCCN)cc2)NC(═O)[C@@H]2CCNC(═O)CC[C@H](NC(═O)[C@H](CO)NC(═O) |
| | (cc3)CNC(═O)CC[C@@H](C(═O)N[C@@H](CCN)C(═O)N[C@@](C) |
| | (Cc3cccnc3)C(N)═O)NC1═O)C(═O)N[C@@H](Cc1c[nH]c3ccccc13)C(═O)N[C@@H] |
| | (CCC(N)═O)C(═O)N2 |
| 4 | [4-Aminomethyl-phenylacetyl](1c)*SE(2c)WQ[Dab](2c)[Y(2-aminoethoxy)][2- |
| | Nal][2-Me-Leu]E(1c)[Dab][N-Me-3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)N(C)[C@@H] |
| | (Cc3cccnc3)C(N)═O)NC1═O)C(═O)N[C@@H](Cc1c[nH]c3ccccc13)C |
| | (═O)N[C@@H](CCC(N)═O)C(═O)N2 |
| 5 | [4-Aminomethyl-phenylacetyl](1c)*SE(2c)WQ[Dab](2c)[Y(2-aminoethoxy)][2- |
| | Nal][2-Me-Leu]E(1c)[Dab][{d}3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)N[C@H] |
| | (Cc3cccnc3)C(N)═O)NC1═O)C(═O)N[C@@H](Cc1c[nH]c3ccccc13)C(═O)N[C@@H] |
| | (CCC(N)═O)C(═O)N2 |
| 6 | [4-Aminomethyl-phenylacetyl](1c)*SE(2c)WQ[Dab](2c)[Y(2-aminoethoxy)][2- |
| | Nal][2-Me-Leu]E(1c)[Dab]-[NH-(4-(pyridin-3-yl)butanyl)] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C |
| | (═O)NCCCCc3cccnc3)NC1═O)C(═O)N[C@@H](Cc1c[nH]c3ccccc13)C(═O)N[C@@H] |
| | (CCC(N)═O)C(═O)N2 |
| 7 | [4-Aminomethyl-phenylacetyl](1c)*SE(2c)WQ[Dab](2c)[Y(2-aminoethoxy)][2- |
| | Nal][2-Me-Leu]E(1c)[Dab]-[NH-(2-(pyridin-3-yl)ethyl)] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)NCCc3cccnc3)NC1═O)C |
| | (═O)N[C@@H](Cc1c[nH]c3ccccc13)C(═O)N[C@@H](CCC(N)═O)C |
| | (═O)N2 |
| 8 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 9 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][Sar]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)N[C@@H] |
| | (Cc3cccnc3)C(═O)N(C)CC(N)═O)NC1═O)C(═O)N[C@@H](Cc1c[nH]c3ccccc13)C |
| | (═O)N[C@@H](CCC(N)═O)C(═O)N2 |
| 10 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][{d}H]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)N[C@@H] |
| | (Cc3cccnc3)C(═O)N[C@H](Cc3cnc[nH]3)C(N)═O)NC1═O)C(═O)N[C@@H] |
| | (Cc1c[nH]c3ccccc13)C(═O)N[C@@H](CCC(N)═O)C(═O)N2 |
| 11 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3-Pyridyl)-Ala][beta-hLeu]-[NH2] |
| | CC(C)C[C@@H](CC(N)═O)NC(═O)[C@H](Cc1cccnc1)NC(═O)[C@H]NC |
| | (═O)[C@@H]1CCC(═O)NCc2ccc(cc2)CC(═O)N[C@@H](CO)C(═O)N[C@ |
| | (CCN)H]2CCC(═O)NCC[C@H](NC(═O)[C@H](CCC(N)═O)NC(═O)[C@H] |
| | (Cc3c[nH]c4ccccc34)NC2═O)C(═O)N[C@@H](Cc2ccc(OCCN)cc2)C(═O)N[C@@H] |
| | (Cc2ccc3ccccc3c2)C(═O)N[C@@](C)(CC(C)C)C(═O)N1 |
| 12 | [4-Aminomethyl-phenylacetyl](1c)*SE(2c)WQ[Dab](2c)[Y(2-aminoethoxy)][2- |
| | Nal][2-Me-Leu]E(1c)[Dab]H[3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)Cc[C@@H](C(═O)N[C@@H](CCN)C(═O)N[C@@H] |
| | (Cc3cnc[nH]3)C(═O)N[C@@H](Cc3cccnc3)C(N)═O)NC1═O)C(═O)N[C@@H] |
| | (Cc1c[nH]c3ccccc13)C(═O)N[C@@H](CCC(N)═O)C(═O)N2 |
| 13 | [4-Aminomethyl-phenylacetyl](1c)*SE(2c)WQ[Dab](2c)[Y(2-aminoethoxy)][2- |
| | Nal][2-Me-Leu]E(1c)[Dab][{d}H]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)Cc[C@@H](C(═O)N[C@@H](CCN)C(═O)N[C@H] |
| | (Cc3cnc[nH]3)C(N)═O)NC1═O)C(═O)N[C@@H](Cc1c[nH]c3ccccc13)C |
| | (═O)N[C@@H](CCC(N)═O)C(═O)N2 |
| 14 | [4-Aminomethyl-phenylacetyl](1c)*SE(2c)WQ[Dab](2c)[Y(2-aminoethoxy)][2- |
| | Nal][2-Me-Leu]E(1c)[Dab][H(Me)]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)N[C@@H](CCN)C(═O)N[C@@H] |
| | (Cc3cn(C)cn3)C(N)═O)NC1═O)C(═O)N[C@@H](Cc1c[nH]c3ccccc13)C(═O)N[C@@H] |
| | (CCC(N)═O)C(═O)N2 |
| 15 | [4-Aminomethyl-phenylacetyl](1c)*SE(2c)WQ[Dab](2c)[Y(2-aminoethoxy)][2- |
| | Nal][2-Me-Leu]E(1c)[Iso-Dab][2-Me-3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)NCC[C@H](N)C(═O)N[C@@](C) |
| | (Cc3cccnc3)C(N)═O)NC1═O)C(═O)N[C@@H](Cc1c[nH]c3ccccc13)C(═O)N[C@@H] |
| | (CCC(N)═O)C(═O)N2 |
| 16 | [4-Aminomethyl-phenylacetyl](1c)*SE(2c)WQ[Dab](2c)[Y(2-aminoethoxy)][2- |
| | Nal][2-Me-Leu]E(1c)[{d}Gly-CF3][3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)NC[C@@H](N[C@@H] |
| | (Cc3cccnc3)C(N)═O)C(F)(F)F)NC1═O)C(═O)N[C@@H](Cc1c[nH]c3ccccc13)C(═O)N[C@@H] |
| | (CCC(N)═O)C(═O)N2 |
| 17 | [4-Aminomethyl-phenylacetyl](1c)*SE(2c)WQ[Dab](2c)[Y(2-aminoethoxy)][2- |
| | Nal][2-Me-Leu]E(1c)[Gly-CF3][3-(3-Pyridyl)-Ala]-[NH2] |
| | CC(C)C[C@]1(C)NC(═O)[C@H](Cc2ccc3ccccc3c2)NC(═O)[C@H](Cc2ccc |
| | (OCCN)cc2)NC(═O)[C@@H]2CCNC(═O)CC[C@H](NC(═O)[C@H](CO)NC |
| | (═O)Cc3ccc(cc3)CNC(═O)CC[C@@H](C(═O)NC[C@H](N[C@@H](Cc3cccnc3)C |
| | (N)═O)C(F)(F)F)NC1═O)C(═O)N[C@@H](Cc1c[nH]c3ccccc13)C(═O)N[C@@H] |
| | (CCC(N)═O)C(═O)N2 |
| 18 | [4-Aminomethyl-phenylacetyl](1c)*[N-Me-Ser]E(2c)WQ[Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N(C)[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 19 | [4-Aminomethyl-phenylacetyl](1c)*SE(2c)WQ[Dab](2c)[Y(2-aminoethoxy)][2- |
| | Nal][2-Me-Leu]E(1c)[Dab][3-(3,5-Pyrimidyl)-Ala]-[NHMe] |
| | CNC(═O)[C@H](Cc1cncnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[c@@] |
| | (C)(CC(C)C)C(═O)N1 |
| 20 | [4-Aminomethyl-phenylacetyl](1c)*AE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](Cc2ccc3ccccc3c2)C(═O)N[C@@] |
| | (C)(CC(C)C)C(═O)N1 |
| 21 | [4-Aminomethyl-phenylacetyl](1c)*FE(2c)WQ[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC@H] |
| | (═O)NCc2ccc(cc2)CC(═O)N[C@@H](Cc2ccccc2)C(═O)N[C@H]2CCC(═O)NCC[C |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 22 | [4-Aminomethyl-phenylacetyl](1c)*SE(2c)WA[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](C)NC(═O)[C@H](Cc3c[nH]c4ccccc34)NC2═O)C(═O)N[C@@H] |
| | (Cc2ccc(OCCN)cc2)C(═O)N[C@@H](Cc2ccc3ccccc3c2)C(═O)N[C@@](C) |
| | (CC(C)C)C(═O)N1 |
| 23 | [4-Aminomethyl-phenylacetyl](1c)*SE(2c)WF[Dab](2c)[Y(2-aminoethoxy)][2- |
| | Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H](NC |
| | (═O)[C@H](Cc3ccccc3)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 24 | [4-Aminomethyl-phenylacetyl](1c)*SE(2c)W[K(Ac)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCCCNC(C)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 25 | [4-Aminomethyl-phenylacetyl](1c)*SE(2c)W[Dab(Ac)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H](NC |
| | (═O)[C@H](CCNC(C)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 26 | [4-Aminomethyl-phenylacetyl](1c)*SE(2c)W[Cit][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCCNC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC)(C)C)C(═O)N1 |
| 27 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(═O)N3CCCC3)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2) |
| 28 | [4-Aminomethyl-phenylacetyl](1c)*SE(2c)W[Orn][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCCN)NC(═O)[C@H](Cc3c[nH]c4ccccc34)NC2═O)C(═O)N[C@@H] |
| | (Cc2ccc(OCCN)cc2)C(═O)N[C@@H](Cc2ccc3ccccc3c2)C(═O)N[C@@ |
| | (C)(CC(C)C)C(═O)N1 |
| 29 | [4-Aminomethyl-phenylacetyl](1c)*SE(2c)W[Q(Me)][Dab](2c)[Y(2- |
| | aminoethoxy)][2-Nal][2-Me-Leu]E(1c)[Dab][3-(3-Pyridyl)-Ala]-[NHMe] |
| | CNC(═O)CC[C@@H]1NC(═O)[C@H](Cc2c[nH]c3ccccc23)NC(═O)[C@@H]2CCC |
| | (═O)NCC[C@H](NC1═O)C(═O)N[C@@H](Cc1ccc(OCCN)cc1)C(═O)N[C@@H] |
| | (Cc1ccc3ccccc3c1)C(═O)N[C@@](C)(CC(C)C)C(═O)N[C@H](C(═O)N[C@@H] |
| | (CCN)C(═O)N[C@@H](Cc1cccnc1)C(═O)NC)CCC(═O)NCc1ccc(cc1)CC |
| | (═O)N[C@@H](CO)C(═O)N2 |
| 30 | [4-Aminomethyl-phenylacetyl](1c)*SE(2c)W[Q(2Me)][Dab](2c)[Y(2- |
| | aminoethoxy)][2-Nal][2-Me-Leu]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](CO)C(═O)N[C@H]2CCC(═O)NCC[C@H](NC |
| | (═O)[C@H](CCC(═O)N(C)C)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 31 | [4-Aminomethyl-phenylacetyl](1c)*SE(2c)WQ[Dab](2c)[Y(2-aminoethoxy)][2- |
| | Nal][2-Me-Leu]E(1c)S[3-(3-Pyridyl)-Ala]-[NHMe] |
| | CNC(═O)[C@H](Cc1cccnc1)NC(═O)[C@H](CO)NC(═O)[C@@H]1CCC |
| | (═O)NCc2ccc(cc2)CC(═O)N[C@@H](CO)C(═O)N[C@H]2CCC(═O)NCC[C@H](NC |
| | (═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 32 | [4-Aminomethyl-phenylacetyl](1c)*SE(2c)WQ[Dab](2c)[Y(2-aminoethoxy)][2- |
| | Nal][2-Me-Leu]E(1c)[Nle][3-(3-Pyridyl)-Ala]-[NHMe] |
| | CCCC[C@H](NC(═O)[C@@H]1CCC(═O)NCc2ccc(cc2)CC(═O)N[C@@H] |
| | (CO)C(═O)N[C@H]2CCC(═O)NCC[C@H](NC(═O)[C@H](CCC(N)═O)NC |
| | (═O)[C@H](Cc3c[nH]c4ccccc34)NC2═O)C(═O)N[C@@H](Cc2ccc(OCCN)cc2)C |
| | (═O)N[C@@H](Cc2ccc3ccccc3c2)C(═O)N[C@@](C)(CC(C)C)C(═O)N1)C |
| | (═O)N[C@@H](Cc1cccnc1)C(═O)NC |
| 33 | [4-Aminomethyl-phenylacetyl](1c)*SE(2c)WQ[Dab](2c)[Y(2-aminoethoxy)][2- |
| | Nal][2-Me-Leu]E(1c)Q[3-(3-Pyridyl)-Ala]-[NHMe] |
| | CNC(═O)[C@H](Cc1cccnc1)NC(═O)[C@H](CCC(N)═O)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 34 | [4-Aminomethyl-phenylacetyl](1c)*SE(2c)WQ[Dab](2c)[Y(2-aminoethoxy)][2- |
| | Nal][2-Me-Leu]E(1c)H[3-(3-Pyridyl)-Ala]-[NHMe] |
| | CNC(═O)[C@H](Cc1cccnc1)NC(═O)[C@H](Cc1cnc[nH]1)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 35 | [4-Aminomethyl-phenylacetyl](1c)*SE(2c)WQ[Dab](2c)[Y(2-aminoethoxy)][2- |
| | Nal][2-Me-Leu]E(1c)[THP][3-(3-Pyridyl)-Ala]-[NHMe] |
| | CNC(═O)[C@H](Cc1cccnc1)NC(═O)C1(NC(═O)[C@@H]2CCC(═O)NCc3ccc |
| | (cc3)CC(═O)N[C@@H](CO)C(═O)N[C@H]3CCC(═O)NCC[C@H](NC(═O)[C@H] |
| | (CCC(N)═O)NC(═O)[C@H](Cc4c[nH]c5ccccc45)NC3═O)C(═O)N[C@@H] |
| | (Cc3ccc(OCCN)cc3)C(═O)N[C@@H](Cc3ccc4ccccc4c3)C(═O)N[C@@](C) |
| | (CC(C)C)C(═O)N2)CCOCC1 |
| 36 | [4-Aminomethyl-phenylacetyl](1c)*SE(2c)WQ[Dab](2c)[Y(2-aminoethoxy)][2- |
| | Nal][2-Me-Leu]E(1c)[hSer][3-(3-Pyridyl)-Ala]-[NHMe] |
| | CNC(═O)[C@H](Cc1cccnc1)NC(═O)[C@H](CCO)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 37 | [4-Aminomethyl-phenylacetyl](1c)*SE(2c)WQ[Dab](2c)[Y(2-aminoethoxy)][2- |
| | Nal][2-Me-Leu]E(1c)[Ser(OMe)][3-(3-Pyridyl)-Ala]-[NHMe] |
| | CNC(═O)[C@H](Cc1cccnc1)NC(═O)[C@H](COC)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 38 | [4-Aminomethyl-phenylacetyl](1c)*SE(2c)WQ[Dab](2c)[Y(2-aminoethoxy)][2- |
| | Nal][2-Me-Leu]E(1c)[{d}S][3-(3-Pyridyl)-Ala]-[NHMe] |
| | CNC(═O)[C@H](Cc1cccnc1)NC(═O)[C@@H](CO)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)N[C@@] |
| | (C)(CC(C)C)C(═O)N1 |
| 39 | [4-Aminomethyl-phenylacetyl](1c)*SE(2c)WQ[Dab](2c)[Y(2-aminoethoxy)][2- |
| | Nal][2-Me-Leu]E(1c)[2-Me-Ser][3-(3-Pyridyl)-Ala]-[NHMe] |
| | CNC(═O)[C@H](Cc1cccnc1)NC(═O)[C@](C)(CO)NC(═O)[C@@H]1CCC |
| | (═O)NCc2ccc(cc2)CC(═O)N[C@@H](CO)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C |
| | (═O)N[C@@](C)(CC(C)C)C(═O)N1 |
| 40 | [4-Aminomethyl-phenylacetyl](1c)*AE(2c)WQ[Dab](2c)[Y(2-aminoethoxy)][2- |
| | Nal][2-Me-Leu]E(1c)S[3-(3-Pyridyl)-Ala]-[NHMe] |
| | CNC(═O)[C@H](Cc1cccnc1)NC(═O)[C@H](CO)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](Cc2ccc3ccccc3c2)C(═O)N[C@@] |
| | (C)(CC(C)C)C(═O)N1 |
| 41 | [4-Aminomethyl-phenylacetyl](1c)*AE(2c)WQ[Dab](2c)[hPhe][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (CCc2ccccc2)C(═O)N[C@@H](Cc2ccc3ccccc3c2)C(═O)NC(C)(C)C(═O)N1 |
| 42 | [4-Aminomethyl-phenylacetyl](1c)*AE(2c)WQ[Dab](2c)[Y(2- |
| | aminoethoxy)][Cyclopropyl-Ala][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](CC2CC2)C(═O)NC(C)(C)C(═O)N1 |
| 43 | [4-Aminomethyl-phenylacetyl](1c)*AE(2c)WQ[Dab](2c)[7-AzaTrp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2c[nH]c3ncccc23)C(═O)N[C@@H](Cc2ccc3ccccc3c2)C(═O)NC(C)(C)C(═O)N1 |
| 44 | [4-Aminomethyl-phenylacetyl](1c)*AE(2c)WQ[Dab](2c)[Y(2-aminoethoxy)][7- |
| | AzaTrp][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](Cc2c[nH]c3ncccc23)C(═O)NC(C)(C)C(═O)N1 |
| 45 | [4-Aminomethyl-phenylacetyl](1c)*AE(2c)WQ[Dab](2c)[beta-hTrp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2c[nH]c3ccccc23)CC(═O)N[C@@H](Cc2ccc3ccccc3c2)C(═O)NC(C)(C)C(═O)N1 |
| 46 | [4-Aminomethyl-phenylacetyl](1c)*AE(2c)WQ[Dab](2c)[Y(2- |
| | aminoethoxy)][beta-hTrp][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](Cc2c[nH]c3ccccc23)CC(═O)NC(C)(C)C(═O)N1 |
| 47 | [4-Aminomethyl-phenylacetyl](1c)*AE(2c)WQ[Dab](2c)[7-F-Trp][2- |
| | Nal][Aib]E(1c)[Dab][3-(3-Pyridyl)-Ala]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2c[nH]c3c(F)cccc23)C(═O)N[C@@H](Cc2ccc3ccccc3c2)C(═O)NC(C)(C)C(═O)N1 |
| 48 | [4-aminomethyl-phenylacetyl(1c)*][N-Me-Ala]E(2c)WQ[Dab](2c)[Y(2- |
| | aminoethoxy)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N(C)[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(OCCN)cc2)C(═O)N[C@@H](Cc2ccc3ccccc3c2)C(═O)NC |
| | (C)(C)C(═O)N1 |
| 49 | 4-aminomethyl-phenylacetyl](1c)*AE(2c)WQ[Dab](2c)[Y(2-aminoethoxy)][3- |
| | quinolinylalanine][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(OCCN)cc2)C(═O)N[C@@H](Cc2cnc3ccccc3c2)C(═O)NC(C)(C)C(═O)N1 |
| 51 | [4-aminomethyl-phenylacetyl](1c)*AE(2c)WQ[Dab](2c)[F(4- |
| | morpholine)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2ccc(N3CCOCC3)cc2)C(═O)N[C@@H](Cc2ccc3ccccc3c2)C(═O)NC(C)(C)C(═O)N1 |
| 52 | [4-aminomethyl-phenylacetyl](1c)*AE(2c)WQ[Dab](2c)[3- |
| | quinolinylalanine][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)N[C@@H](C)C(═O)N[C@H]2CCC(═O)NCC[C@H](NC |
| | (═O)[C@H](CCC(N)═O)NC(═O)[C@H](Cc3c[nH]c4ccccc34)NC2═O)C(═O)N[C@@H] |
| | (Cc2cnc3ccccc3c2)C(═O)N[C@@H](Cc2ccc3ccccc3c2)C(═O)NC(C) |
| | (C)C(═O)N1 |
| 53 | [4-aminomethyl-phenylacetyl](1c)*AE(2c)WQ[Dab](2c)[F(4- |
| | morpholine)][2Nal][Aib]E(1c)[Ser(OCH3)][3-(3-Pyridyl)-alanyl]-[NHMe] |
| | CNC(═O)[C@H](Cc1cccnc1)NC(═O)[C@H](COC)NC(═O)[C@@H]1CCC |
| | (═O)NCc2ccc(cc2)CC(═O)N[C@@H](C)C(═O)N[C@H]2CCC(═O)NCC[C@H] |
| | (NC(═O)[C@H](CCC(N)═O)NC(═O)[C@H](Cc3c[nH]c4ccccc34)NC2═O)C |
| | (═O)N[C@@H](Cc2ccc(N3CCOCC3)cc2)C(═O)N[C@@H](Cc2ccc3ccccc3c2)C |
| | (═O)NC(C)(C)C(═O)N1 |
| 54 | [4-aminomethyl-phenylacetyl](1c)*[3-Aminopropanoyl]E(2c)[7-Me- |
| | Trp][Q(2Me)][Dab](2c)[Y(2-aminoethoxy)(N(Me)2)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)NCCC(═O)N[C@H]2CCC(═O)NCC[C@H](NC(═O)[C@H] |
| | (CCC(═O)N(C)C)NC(═O)[C@H](Cc3c[nH]c4c(C)cccc34)NC2═O)C(═O)N[C@@H] |
| | (Cc2ccc(OCCN(C)C)cc2)C(═O)N[C@@H](Cc2ccc3ccccc3c2)C(═O)NC |
| | (C)(C)C(═O)N1 |
| 55 | [4-aminomethyl-phenylacetyl](1c)*[3-Aminopropanoyl]E(2c)[7-Me- |
| | Trp][Q(2Me)][Dab](2c)[Y(nPentylamine)(N⁺(Me)3)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)NCCC(═O)N[C@H]2CCC(═O)NCC[C@H](NC(═O)[C@H] |
| | (CCC(═O)N(C)C)NC(═O)[C@H](Cc3c[nH]c4c(C)cccc34)NC2═O)C(═O)N[C@@H] |
| | (Cc2ccc(OCCCCC[N+](C)(C)C)cc2)C(═O)N[C@@H](Cc2ccc3ccccc3c2)C |
| | (═O)NC(C)(C)C(═O)N1 |
| 56 | [4-aminomethyl-phenylacetyl](1c)*[3-Aminopropanoyl]E(2c)[7-Me- |
| | Trp][Q(2Me)][Dab](2c)[Tyr(2-trimethyl-PEG3)][2Nal][Aib]E(1c)[Dab][3-(3- |
| | Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)NCCC(═O)N[C@H]2CCC(═O)NCC[C@H](NC(═O)[C@H] |
| | (CCC(═O)N(C)C)NC(═O)[C@H](Cc3c[nH]c4c(C)cccc34)NC2═O)C(═O)N[C@@H] |
| | (Cc2ccc(OCCOC—COCC[N+](C)(C)C)cc2)C(═O)N[C@@H](Cc2ccc3ccccc3c2)C(═O)NC(C) |
| | (C)C(═O)N1 |
| 57 | [4-aminomethyl-phenylacetyl](1c)*[3-Aminopropanoyl]E(2c)[7-Me- |
| | Trp][K(NMePEG3)][Dab](2c)[Y(2- |
| | aminoethoxy)(N(Me)2)][2Nal][Aib]E(1c)[Dab][3-(3-Pyridyl)-alanyl]-[NHMe] |
| | CNC(═O)[C@H](Cc1cccnc1)NC(═O)[C@H](CCN)NC(═O)[C@@H]1CCC |
| | (═O)NCc2ccc(cc2)CC(═O)NCCC(═O)N[C@H]2CCC(═O)NCC[C@H](NC(═O)[C@H] |
| | (CCCCN(C)C(═O)COC—COCC[N+](C)(C)C)NC(═O)[C@H](Cc3c[H]c4c(C)cccc34)NC2═O)C |
| | (═O)N[C@@H](Cc2ccc(OCCN(C)C)cc2)C(═O)N[C@@H](Cc2ccc3ccccc3c2)C(═O)NC |
| | (C)(C)C(═O)N1 |

or a pharmaceutically acceptable salt or solvate thereof;
wherein:
* denotes that the (1c), (2a), (2c), and (4c) bridges use the peptide backbone amine or carboxylic acid at the N- or C-terminus, not the side chain amine or carboxylic acid,
(1c) denotes the [2,11] lactam bridge; (2a) denotes the [4,7] 1,3-dithio-propan-2-one bridge; (2c) denotes the [4,7] lactam bridge; (4c) denotes the [1,11] lactam bridge.

25. A pharmaceutical composition comprising a compound according to any one of the preceding claims in combination with a pharmaceutically acceptable carrier, excipient or vehicle.

26. The pharmaceutical composition according to claim 25, wherein said pharmaceutical composition is for oral administration.

27. A method for the synthesis of a compound according to any one of claims 1 to 24, comprising synthesising the analogue by solid-phase or liquid-phase peptide synthesis methodology, optionally isolating and/or purifying the final product, and optionally further comprising the step of forming an amide bond between the amino acid residues at position X1 or at position X2 when X1 is absent and at position X11, and optionally further comprising the step of forming an amide bond or forming two thioether bonds with a linker between the amino acid residues at positions X4 and X7.

28. A compound according to any one of claims 1 to 24, or a pharmaceutical composition according to any of claims 25-26, for use in a method of medical treatment.

29. A compound according to any one of claims 1 to 24, or a pharmaceutical composition according to any of claims 25-26, for use in a method of prevention or treatment of inflammatory bowel disease (IBD), psoriasis, psoriatic arthritis, and combinations thereof.

30. The compound or pharmaceutical composition for use according to claim 29, wherein the IBD is selected from Crohn's Disease or ulcerative colitis.


SEQUENCE LISTING (240 sequences)

SEQ ID NO: 1 - protein, 12 aa, synthetic construct
  CROSSLNK 1..11: note = lactam bridge
  CROSSLNK 4..7: note = 1,3-dithio-propan-2-one bridge
  SITE 1: note = 3-Aminopropanoyl
  SITE 2: note = 3-(3-pyridyl)-L-alanine
  SITE 8: note = (2S)-2-amino-3-[4-(2-aminoethoxy)phenyl]propanoic acid
  SITE 9: note = 3-(2-naphthyl)-L-alanine
  SITE 10: note = 2-methyl-L-leucine
  SITE 12: note = D-arginine
XXSCWQCXXXER

SEQ ID NO: 2 - protein, 12 aa, synthetic construct
  CROSSLNK 1..11: note = lactam bridge
  CROSSLNK 4..7: note = 1,3-dithio-propan-2-one bridge
  SITE 1: note = 3-(3-pyridyl)-L-alanine
  SITE 2: note = 3-Aminopropanoyl
  SITE 8: note = (2S)-2-amino-3-[4-(2-aminoethoxy)phenyl]propanoic acid
  SITE 9: note = 3-(2-naphthyl)-L-alanine
  SITE 10: note = 2-methyl-L-leucine
  SITE 12: note = D-arginine
XXSCWQCXXXER

SEQ ID NO: 3 - protein, 12 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 2-Me-3-(3-Pyridyl)-Ala
XSEWQXXXXEXX

SEQ ID NO: 4 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = N-Me-3-(3-Pyridyl)-Ala
XSEWQXXXXEXX

SEQ ID NO: 5 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(3-pyridyl)-D-alanine
XSEWQXXXXEXX

SEQ ID NO: 6 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
XSEWQXXXXEX

SEQ ID NO: 7 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
XSEWQXXXXEX

SEQ ID NO: 8 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XSEWQXXXXEXX

SEQ ID NO: 9 - protein, 13 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 13: note = MeGly
XSEWQXXXXEXXX

SEQ ID NO: 10 - protein, 13 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 13: note = D-histidine
XSEWQXXXXEXXH

SEQ ID NO: 11 - protein, 13 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 13: note = beta-homoleucine
XSEWQXXXXEXXX

SEQ ID NO: 12 - protein, 13 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 13: note = 3-(3-pyridyl)-L-alanine
XSEWQXXXXEXHX

SEQ ID NO: 13 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = d-histidine
XSEWQXXXXEXH

SEQ ID NO: 14 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 1-methyl-L-histidine
XSEWQXXXXEXX

SEQ ID NO: 15 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = iso-(2S)-2,4-diaminobutanoic acid
  SITE 12: note = 2-Me-3-(3-Pyridyl)-Ala
XSEWQXXXXEXX

SEQ ID NO: 16 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-glycine-CF3
  SITE 12: note = 3-(3-pyridyl)-L-alanine
XSEWQXXXXEXX

SEQ ID NO: 17 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = glycine-CF3
  SITE 12: note = 3-(3-pyridyl)-L-alanine
XSEWQXXXXEXX

SEQ ID NO: 18 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 2: note = N-methyl-serine
XXEWQXXXXEXX

SEQ ID NO: 19 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3,5-Pyrimidyl)-Ala
XSEWQXXXXEXX

SEQ ID NO: 20 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XAEWQXXXXEXX

SEQ ID NO: 21 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XFEWQXXXXEXX

SEQ ID NO: 22 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XSEWAXXXXEXX

SEQ ID NO: 23 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XSEWFXXXXEXX

SEQ ID NO: 24 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = acetyllysine
XSEWXXXXXEXX

SEQ ID NO: 25 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = acetyl-(2S)-2,4-diaminobutanoic acid
XSEWXXXXXEXX

SEQ ID NO: 26 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = citrulline
XSEWXXXXXEXX

SEQ ID NO: 27 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = Q(pyrrolidin) also known as: (2S)-2-amino-5-oxo-5-pyrrolidin-1-yl-pentanoic acid
XSEWXXXXXEXX

SEQ ID NO: 28 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = Orn also known as: L-ornithine or (2S)-2,5-diaminopentanoic acid
XSEWXXXXXEXX

SEQ ID NO: 29 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = N5-L-Methylglutamine
XSEWXXXXXEXX

SEQ ID NO: 30 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
  SITE 5: note = N,N, dimethyl--L-glutamine
XSEWXXXXXEXX

SEQ ID NO: 31 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XSEWQXXXXESX

SEQ ID NO: 32 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = Nle also known as: L-norleucine or (2S)-2-aminohexanoic acid
  SITE 12: note = 3-(3-pyridyl)-L-alanine
XSEWQXXXXEXX

SEQ ID NO: 33 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(3-pyridyl)-L-alanine
XSEWQXXXXEQX

SEQ ID NO: 34 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(3-pyridyl)-L-alanine
XSEWQXXXXEHX

SEQ ID NO: 35 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = THP also known as: 4-aminotetrahydropyran-4-carboxylic acid
  SITE 12: note = 3-(3-pyridyl)-L-alanine
XSEWQXXXXEXX

SEQ ID NO: 36 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = homoserine
  SITE 12: note = 3-(3-pyridyl)-L-alanine
XSEWQXXXXEXX

SEQ ID NO: 37 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = Ser(OMe) also known as: O-methyl-L-serine or (2S)-2-amino-3-methoxy-propanoic acid
  SITE 12: note = 3-(3-pyridyl)-L-alanine
XSEWQXXXXEXX

SEQ ID NO: 38 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-serine
  SITE 12: note = 3-(3-pyridyl)-L-alanine
XSEWQXXXXESX

SEQ ID NO: 39 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = 2-methyl-serine
  SITE 12: note = 3-(3-pyridyl)-L-alanine
XSEWQXXXXEXX

SEQ ID NO: 40 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 12: note = 3-(3-pyridyl)-L-alanine
XAEWQXXXXESX

SEQ ID NO: 41 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = homo-phenylalanine
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 42 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = Cyclopropyl-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 43 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = 7-Aza-L-tryptophan
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 44 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 7-Aza-L-tryptophan
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 45 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = beta-homo-tryptophan
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 46 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = beta-homo-tryptophan
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 47 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = 7-F-tryptophan
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 48 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
  SITE 2: note = N-methyl-alanine
XXEWQXXXXEXX

SEQ ID NO: 49 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-quinolinylalanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 50 - skipped

SEQ ID NO: 51 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = F(4-morpholine) also known as: 4-morpholine-L-phenylalanine or (2S)-2-Amino-3-(4-morpholin-4-ylphenyl)propanoic Acid
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XSEWQXXXXEXX

SEQ ID NO: 52 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = 3-quinolinylalanine
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = (2S)-2,4-diaminobutanoic acid
  SITE 12: note = 3-(3-pyridyl)-L-alanine
XAEWQXXXXEXX

SEQ ID NO: 53 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = F(4-morpholine) also known as: 4-morpholine-L-phenylalanine or (2S)-2-Amino-3-(4-morpholin-4-ylphenyl)propanoic Acid
  SITE 8: note = 3-(2-naphthyl)-L-alanine
  SITE 9: note = Aib also known as: 2-aminoisobutyric acid, alpha-methylalanine, - methylalanine, or 2-methylalanine, or 2-amino-2-methylpropanoic acid
  SITE 11: note = Ser(OCH3)
  SITE 12: note = 3-(3-pyridyl)-L-alanine
XAEWQXXXXEXX

SEQ ID NO: 54-57 - skipped (4 entries)

SEQ ID NO: 58 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  MOD_RES 11: note = C-terminal amidation
XSCWQCXXXER

SEQ ID NO: 59 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-arginine; note = C-terminal amidation
XSCWQCXXXER

SEQ ID NO: 60 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
XSCWQCXXXES

SEQ ID NO: 61 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
XSCWQCXXXEX

SEQ ID NO: 62 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-arginine; note = C-terminal amidation
XTCWQCXXXER

SEQ ID NO: 63 - protein, 11 aa, synthetic construct
  CROSSLNK 1..10: note = lactam bridge
  CROSSLNK 3..6: note = lactam bridge
  SITE 1: note = (2S)-2,4-diaminobutanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = D-arginine
XSCWQCXXXER

SEQ ID NO: 64 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-NH2-(2-(trimethyl-2-aminoethoxy)ethoxy)propyl)
  SITE 8: note = 3-(2-naphthyl)-L-alanine
  SITE 9: note = 2-methyl-L-leucine
  SITE 11: note = D-arginine; note = C-terminal amidation
XSCWQCXXXER

SEQ ID NO: 65 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-glutamine; note = C-terminal amidation
XSCWQCXXXEQ

SEQ ID NO: 66 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 4-Aminomethyl-phenylacetyl
  SITE 8: note = 3-(2-naphthyl)-L-alanine
  SITE 9: note = 2-methyl-L-leucine
  SITE 11: note = C-terminal amidation; note = D-glutamic acid
XSCWQCXXXEE

SEQ ID NO: 67 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 4-Aminomethyl-phenylacetyl
  SITE 8: note = 3-(2-naphthyl)-L-alanine
  SITE 9: note = 2-methyl-L-leucine
  SITE 11: note = D-histidine; note = C-terminal amidation
XSCWQCXXXEH

SEQ ID NO: 68 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 4-Aminomethyl-phenylacetyl
  SITE 8: note = 3-(2-naphthyl)-L-alanine
  SITE 9: note = 2-methyl-L-leucine
  SITE 11: note = C-terminal amidation; note = 3-Aminopropanoyl
XSCWQCXXXEX

SEQ ID NO: 69 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation; note = 3-(3-pyridyl)-L-alanine
  SITE 11: note = (2S)-2,4-diaminobutanoic acid
XSCWQCXXXEXX

SEQ ID NO: 70 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 6-F-Trp
  SITE 9: note = 2-methyl-L-leucine
  SITE 11: note = (2S)-2,4-diaminobutanoic acid
  SITE 12: note = 3-(3-pyridyl)-L-alanine; note = C-terminal amidation
XSCWQCXXXEXX

SEQ ID NO: 71 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 6: note = (2S)-2,4-diaminobutanoic acid
  SITE 12: note = 3-(3-pyridyl)-L-alanine; note = C-terminal amidation
XSEWQXXXXEXX

SEQ ID NO: 72 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 3: note = (2S)-2,4-diaminobutanoic acid
  SITE 12: note = C-terminal amidation; note = 3-(3-pyridyl)-L-alanine
XSXWQEXXXEXX

SEQ ID NO: 73 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid
  SITE 6: note = Orn also known as:also known as: L-ornithine or (2S)-2,5-diaminopentanoic acid
  SITE 12: note = C-terminal amidation; note = 3-(3-pyridyl)-L-alanine
XSEWQXXXXEXX

SEQ ID NO: 74 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 6: note = (2S)-2,4-diaminobutanoic acid
XSEWQXXXXEX

SEQ ID NO: 75 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
XVEWQXXXXEX

SEQ ID NO: 76 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
XIEWQXXXXEX

SEQ ID NO: 77 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
XSEWVXXXXEX

SEQ ID NO: 78 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
XSEWHXXXXEX

SEQ ID NO: 79 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 6: note = (2S)-2,4-diaminobutanoic acid
XSEWHXFXXEX

SEQ ID NO: 80 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 6: note = (2S)-2,4-diaminobutanoic acid
XSEWQXXXXEX

SEQ ID NO: 81 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = F(3-F)
  SITE 9: note = 2-methyl-L-leucine
  SITE 11: note = (2S)-2,4-diaminobutanoic acid; note = C-terminal amidation
  SITE 6: note = (2S)-2,4-diaminobutanoic acid
XSEWQXXXXEX

SEQ ID NO: 82 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = F(3,4-Me)
  SITE 9: note = 2-methyl-L-leucine
  SITE 11: note = (2S)-2,4-diaminobutanoic acid; note = C-terminal amidation
  SITE 6: note = (2S)-2,4-diaminobutanoic acid
XSEWQXXXXEX

SEQ ID NO: 83 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-valine
  SITE 11: note = C-terminal amidation; note = (2S)-2,4-diaminobutanoic acid
  SITE 6: note = (2S)-2,4-diaminobutanoic acid
XSEWQXXXXEX

SEQ ID NO: 84 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
  SITE 4: note = 4-F-Trp
XSEXQXXXXEX

SEQ ID NO: 85 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 6: note = (2S)-2,4-diaminobutanoic acid
  SITE 4: note = 7-F-Trp also known as: (2S)-2-amino-3-(7-fluoro-1H-indol-3-yl)propanoic acid
XSEXQXXXXEX

SEQ ID NO: 86 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 2: note = N-methyl-serine
XXCWQCXXXEX

SEQ ID NO: 87 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 3: note = N-methyl-cysteine
XSXWQCXXXEX

SEQ ID NO: 88 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 4: note = N-methyl-Trp
XSCXQCXXXEX

SEQ ID NO: 89 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 5: note = N-methyl-glutamine
XSCWXCXXXEX

SEQ ID NO: 90 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = (2S)-2,4-diaminobutanoic acid; note = C-terminal amidation
  SITE 2: note = Q(pyrrolidin) also known as: (2S)-2-amino-5-oxo-5-pyrrolidin-1-yl-pentanoic acid
  SITE 6: note = (2S)-2,4-diaminobutanoic acid
XXEWQXXXXEX

SEQ ID NO: 91 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation; note = (2S)-2,4-diaminobutanoic acid
  SITE 5: note = Q(pyrrolidin) also known as: (2S)-2-amino-5-oxo-5-pyrrolidin-1-yl-pentanoic acid
  SITE 6: note = (2S)-2,4-diaminobutanoic acid
XSEWXXXXXEX

SEQ ID NO: 92 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(3-pyridyl)-L-alanine; note = C-terminal amidation
XSCWQCXXXERX

SEQ ID NO: 93 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-valine
  SITE 12: note = 3-(3-pyridyl)-L-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 94 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = F(3,4-Me)
  SITE 9: note = 2-methyl-L-valine
  SITE 12: note = 3-(3-pyridyl)-L-alanine; note = C-terminal amidation
  SITE 6: note = (2S)-2,4-diaminobutanoic acid
XIEWQXXXXEGX

SEQ ID NO: 95 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)
  SITE 9: note = 2-methyl-L-valine
  SITE 12: note = C-terminal amidation; note = 3-(3-pyridyl)-L-alanine
  SITE 6: note = (2S)-2,4-diaminobutanoic acid
XIEWQXXXXEGX

SEQ ID NO: 96 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)
  SITE 9: note = 2-methyl-L-valine
  SITE 12: note = C-terminal amidation; note = 3-(3-pyridyl)-L-alanine
  SITE 6: note = (2S)-2,4-diaminobutanoic acid
  SITE 11: note = 4-aminobutanoic acid
XIEWQXXXXEXX

SEQ ID NO: 97 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)
  SITE 9: note = 2-methyl-L-valine
  SITE 12: note = C-terminal amidation; note = 2,3-Diaminopropanoyl(3-pyridylacetyl
  SITE 6: note = (2S)-2,4-diaminobutanoic acid
XIEWQXXXXEGX

SEQ ID NO: 98 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)
  SITE 9: note = 2-methyl-L-valine
  SITE 12: note = C-terminal amidation; note = 2,3-Diaminopropanoyl(3-pyridylpropionyl)
  SITE 6: note = (2S)-2,4-diaminobutanoic acid
XIEWQXXXXEGX

SEQ ID NO: 99 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)]
  SITE 9: note = 2-methyl-L-valine
  SITE 12: note = 2,3-Diaminopropanoyl(3-fluorobenzoyl); note = C-terminal amidation
  SITE 6: note = (2S)-2,4-diaminobutanoic acid
XIEWQXXXXEGX

SEQ ID NO: 100 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = F(3,4-Me)
  SITE 9: note = 2-methyl-L-valine
  SITE 12: note = 2,3-Diaminopropanoyl(3-fluorophenylacetyl); note = C-terminal amidation
  SITE 6: note = (2S)-2,4-diaminobutanoic acid
XIEWQXXXXEGX

SEQ ID NO: 101 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = F(3,4-Me)
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 102 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = Y(Me)
  SITE 8: note = F(3,4-Me)
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 103 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 2-Me-Phe
  SITE 8: note = F(3,4-Me)
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 104 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = Bip
  SITE 8: note = F(3,4-Me)
  SITE 9: note = 2-methyl-L-valine
  SITE 12: note = 2-Me-3-(3-Pyridyl)-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 105 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 2-Me-F(4-F)
  SITE 8: note = F(3,4-Me)
  SITE 9: note = 2-methyl-L-valine
  SITE 12: note = 2-Me-3-(3-Pyridyl)-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 106 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = D-F(4-F)
  SITE 8: note = F(3,4-Me)
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 107 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Cl)
  SITE 8: note = F(3,4-Me)
  SITE 9: note = 2-methyl-L-valine
  SITE 12: note = 2-Me-3-(3-Pyridyl)-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 108 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = alpha-Me-Trp
  SITE 8: note = F(3,4-Me)
  SITE 9: note = 2-methyl-L-valine
  SITE 12: note = 2-Me-3-(3-Pyridyl)-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 109 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = 3,3-Diphenyl-alanine
  SITE 8: note = F(3,4-Me)
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 110 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = 1-Me-Trp
  SITE 9: note = 2-methyl-L-valine
  SITE 11: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 111 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = Bip
  SITE 9: note = 2-methyl-L-valine
  SITE 12: note = C-terminal amidation; note = 2-Me-3-(3-Pyridyl)-alanine
  SITE 6: note = (2S)-2,4-diaminobutanoic acid
  SITE 11: note = (2S)-2,4-diaminobutanoic acid
XIEWQXXXXEXX

SEQ ID NO: 112 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = F(4-Me)
  SITE 8: note = D-6-F-Trp
  SITE 9: note = 2-methyl-L-valine
  SITE 12: note = 2-Me-3-(3-Pyridyl)-alanine; note = C-terminal amidation
  SITE 6: note = (2S)-2,4-diaminobutanoic acid
  SITE 11: note = (2S)-2,4-diaminobutanoic acid
XIEWQXXXXEXX

SEQ ID NO: 113 - protein, 12 aa, synthetic construct
  CROSSLNK 2..11: note = 1,3-dithio-propan-2-one bridge
  CROSSLNK 3..6: note = 1,3-dithio-propan-2-one bridge
  SITE 10: note = 2-methyl-L-leucine
  SITE 12: note = C-terminal amidation
DCSCWQCWWXCR

SEQ ID NO: 114 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 12: note = C-terminal amidation
LCSCWQCWWLCR

SEQ ID NO: 115 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 12: note = C-terminal amidation
DCSCWECWWLCR

SEQ ID NO: 116 - protein, 12 aa, synthetic construct
  CROSSLNK 2..11: note = lactam bridge
  CROSSLNK 4..7: note = 1,3-dithio-propan-2-one bridge
  SITE 12: note = C-terminal amidation
DESCWQCWWLKR

SEQ ID NO: 117 - protein, 12 aa, synthetic construct
  CROSSLNK 2..11: note = 1,3-dithio-propan-2-one bridge
  CROSSLNK 4..7: note = lactam bridge
  SITE 12: note = C-terminal amidation
DCSEWQKWWLCR

SEQ ID NO: 118 - protein, 10 aa, synthetic construct
  CROSSLNK 1..10: note = lactam bridge
  CROSSLNK 3..6: note = 1,3-dithio-propan-2-one bridge
DSCWQCWWLR

SEQ ID NO: 119 - protein, 12 aa, synthetic construct
  CROSSLNK 2..11: note = lactam bridge
  CROSSLNK 4..7: note = 1,3-dithio-propan-2-one bridge
  SITE 12: note = C-terminal amidation
  SITE 1: note = N-terminal acetylation
DKSCWQCWWLER

SEQ ID NO: 120 - protein, 12 aa, synthetic construct
  CROSSLNK 2..11: note = lactam bridge
  CROSSLNK 4..7: note = 1,3-dithio-propan-2-one bridge
  SITE 12: note = C-terminal amidation
  SITE 1: note = N-terminal acetylation
  SITE 2: note = Orn also known as: L-ornithine or (2S)-2,5-diaminopentanoic acid
DXSCWQCWWLER

SEQ ID NO: 121 - protein, 12 aa, synthetic construct
  CROSSLNK 2..11: note = lactam bridge
  CROSSLNK 4..7: note = 1,3-dithio-propan-2-one bridge
  SITE 11: note = (2S)-2,4-diaminobutanoic acid
  SITE 12: note = C-terminal amidation
DESCWQCWWLXR

SEQ ID NO: 122 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
ESCWQCWWLKR

SEQ ID NO: 123 - protein, 11 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 11: note = C-terminal amidation
DESCWQCWWLK

SEQ ID NO: 124 - protein, 10 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 10: note = C-terminal amidation
ESCWQCWWLK

SEQ ID NO: 125 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
DESCAQCWWLKR

SEQ ID NO: 126 - protein, 12 aa, synthetic construct
  CROSSLNK 2..11: note = lactam bridge
  CROSSLNK 4..7: note = 1,3-dithio-propan-2-one bridge
  SITE 11: note = N-terminal acetylation
  SITE 12: note = C-terminal amidation
DESCWQCAWLKR

SEQ ID NO: 127 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
DESCWQCWALKR

SEQ ID NO: 128 - protein, 12 aa, synthetic construct
  CROSSLNK 2..11: note = lactam bridge
  CROSSLNK 4..7: note = 1,3-dithio-propan-2-one bridge
  SITE 5: note = 7-Me-Trp also known as: (2S)-2-amino-3-(7-methyl-1H-indol-3-yl)propanoic acid
  SITE 12: note = C-terminal amidation
  SITE 1: note = N-terminal acetylation
DESCXQCWWLKR

SEQ ID NO: 129 - protein, 12 aa, synthetic construct
  CROSSLNK 2..11: note = lactam bridge
  CROSSLNK 4..7: note = 1,3-dithio-propan-2-one bridge
  SITE 9: note = 3-(2-naphthyl)-L-alanine
  SITE 12: note = C-terminal amidation
  SITE 1: note = N-terminal acetylation
DESCWQCWXLKR

SEQ ID NO: 130 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 8: note = 3-(2-naphthyl)-L-alanine
KSCWQCWXLER

SEQ ID NO: 131 - protein, 12 aa, synthetic construct
  CROSSLNK 2..11: note = lactam bridge
  CROSSLNK 4..7: note = 1,3-dithio-propan-2-one bridge
  SITE 9: note = 3-(2-naphthyl)-L-alanine
  SITE 12: note = C-terminal amidation
  SITE 1: note = N-terminal acetylation
DKSCWQCWXLER

SEQ ID NO: 132 - protein, 12 aa, synthetic construct
  CROSSLNK 2..11: note = lactam bridge
  CROSSLNK 4..7: note = 1,2-phenylenedimethanethiol bridge
  SITE 1: note = N-terminal acetylation
  SITE 12: note = C-terminal amidation
  SITE 9: note = 3-(2-naphthyl)-L-alanine
DKSCWQCWXLER

SEQ ID NO: 133 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 9: note = 3-(2-naphthyl)-L-alanine
DKSCWQCWXLER

SEQ ID NO: 134 - protein, 12 aa, synthetic construct
  CROSSLNK 2..11: note = lactam bridge
  CROSSLNK 4..7: note = 1,4-phenylenedimethanethiol bridge
  SITE 1: note = N-terminal acetylation
  SITE 12: note = C-terminal amidation
  SITE 9: note = 3-(2-naphthyl)-L-alanine
DKSCWQCWXLER

SEQ ID NO: 135 - protein, 12 aa, synthetic construct
  CROSSLNK 2..11: note = lactam bridge
  CROSSLNK 4..7: note = 1,4-disubstituted 1,2,3-triazole bridge
  SITE 1: note = N-terminal acetylation
  SITE 12: note = C-terminal amidation
  SITE 9: note = 3-(2-naphthyl)-L-alanine
  SITE 4: note = K(N3)
  SITE 7: note = L-Propargylglycine
DKSXWQXWXLER

SEQ ID NO: 136 - protein, 12 aa, synthetic construct
  CROSSLNK 2..11: note = 1,3-dithio-propan-2-one bridge
  CROSSLNK 4..7: note = 1,4-disubstituted 1,2,3-triazole bridge
  SITE 1: note = N-terminal acetylation
  SITE 12: note = C-terminal amidation
  SITE 9: note = 3-(2-naphthyl)-L-alanine
  SITE 4: note = azidohomo-L-alanine
  SITE 7: note = L-propargylglycine
DKSXWQXWXLER

SEQ ID NO: 137 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 4: note = 3-(2-naphthyl)-L-alanine
  SITE 8: note = 3-(2-naphthyl)-L-alanine
KSCXQCWXLER

SEQ ID NO: 138 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 4: note = biphenyl-L-alanine
  SITE 8: note = 3-(2-naphthyl)-L-alanine
KSCXQCWXLER

SEQ ID NO: 139 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 4: note = 1-methyl-L-tryptophan
  SITE 8: note = 3-(2-naphthyl)-L-alanine
KSCXQCWXLER

SEQ ID NO: 140 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 11: note = C-terminal amidation
  SITE 8: note = 3-(2-naphthyl)-L-alanine
  SITE 9: note = 3-(2-naphthyl)-L-alanine
DKSCWQCXXLER

SEQ ID NO: 141 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
KSCWQCXXLER

SEQ ID NO: 142 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 8: note = 3-(2-naphthyl)-L-alanine
KSCWQCHXLER

SEQ ID NO: 143 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 8: note = F(4-F)
KSCWQCWXLER

SEQ ID NO: 144 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 8: note = 1-methyl-L-tryptophan
KSCWQCWXLER

SEQ ID NO: 145 - protein, 11 aa, synthetic construct
  CROSSLNK 1..10: note = 1,3-dithio-propan-2-one bridge
  CROSSLNK 3..6: note = 1,3-dithio-propan-2-one bridge
  SITE 1: note = N-terminal acetylation
  SITE 11: note = C-terminal amidation
  SITE 9: note = 2-methyl-L-lysine
CTCWECWWXCS

SEQ ID NO: 146 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 10: note = 2-methyl-L-leucine
GKTCWECWWXES

SEQ ID NO: 147 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 1: note = N-terminal acetylation
  SITE 12: note = C-terminal amidation
  SITE 9: note = 3-(2-naphthyl)-L-alanine
  SITE 10: note = 2-methyl-L-leucine
GCTCWECWXXCR

SEQ ID NO: 148 - protein, 11 aa, synthetic construct
  CROSSLNK 1..10: note = lactam bridge
  CROSSLNK 3..6: note = 1,3-dithio-propan-2-one bridge
  SITE 1: note = 3-aminopropionic acid
  SITE 11: note = C-terminal amidation
  SITE 8: note = 3-(2-naphthyl)-L-alanine
XSCWQCWXLER

SEQ ID NO: 149 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 8: note = 3-(2-naphthyl)-L-alanine
XSCWQCWXLER

SEQ ID NO: 150 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCWQCXXXER

SEQ ID NO: 151 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
XSCWQCXXLER

SEQ ID NO: 152 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 8: note = 3-(2-naphthyl)-L-alanine
  SITE 9: note = 2-methyl-L-leucine
XSCWQCWXXER

SEQ ID NO: 153 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-arginine
XSCWQCXXXER

SEQ ID NO: 154 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = 2-methyl-arginine
XSCWQCXXXEX

SEQ ID NO: 155 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = N-methyl-arginine
XSCWQCXXXEX

SEQ ID NO: 156 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCWQCXXXES

SEQ ID NO: 157 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = F(4-NH2)
XSCWQCXXXEX

SEQ ID NO: 158 - protein, 11 aa, synthetic construct
  CROSSLNK 1..10: note = lactam bridge
  CROSSLNK 3..6: note = 1,3-dithio-propan-2-one bridge
  SITE 10: note = 2-methyl-L-leucine
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = D-leucine
XSCWQCXXLEY

SEQ ID NO: 159 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
KSCWQCXXXER

SEQ ID NO: 160 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = D-leucine
KSCWQCXXLER

SEQ ID NO: 161 - protein, 11 aa, synthetic construct
  CROSSLNK 1..10: note = lactam bridge
  CROSSLNK 3..6: note = 1,3-dithio-propan-2-one bridge
  SITE 1: note = (4-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCWQCXXXER

SEQ ID NO: 162 - protein, 11 aa, synthetic construct
  CROSSLNK 1..10: note = lactam bridge
  CROSSLNK 3..6: note = 1,3-dithio-propan-2-one bridge
  SITE 1: note = 4-(2-Aminoethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCWQCXXXER

SEQ ID NO: 163 - protein, 11 aa, synthetic construct
  CROSSLNK 1..10: note = lactam bridge
  CROSSLNK 3..6: note = 1,3-dithio-propan-2-one bridge
  SITE 1: note = 3-(4-Aminophenyl)propanoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCWQCXXXER

SEQ ID NO: 164 - protein, 12 aa, synthetic construct
  CROSSLNK 2..11: note = 1,3-dithio-propan-2-one bridge
  DISULFID 4..7: note = intrachain
  SITE 12: note = C-terminal amidation
DCSCWQCWWLCR

SEQ ID NO: 165 - protein, 11 aa, synthetic construct
  CROSSLNK 1..10: note = lactam bridge
  DISULFID 3..6: note = intrachain
  SITE 1: note = N-terminal acetylation
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
KSCWQCXXLER

SEQ ID NO: 166 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 10: note = 2-methyl-L-leucine
DEQCWQCYWXKR

SEQ ID NO: 167 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 8: note = 3-(2-naphthyl)-L-alanine
  SITE 10: note = 2-methyl-L-leucine
DEQCWQCXWXKR

SEQ ID NO: 168 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 9: note = 3-(2-naphthyl)-L-alanine
  SITE 10: note = 2-methyl-L-leucine
DETCWQCWXXKR

SEQ ID NO: 169 - protein, 14 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 14: note = C-terminal amidation
  SITE 9: note = 3-(2-naphthyl)-L-alanine
  SITE 10: note = 2-methyl-L-leucine
DESCWQCWXXKENG

SEQ ID NO: 170 - protein, 13 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 13: note = C-terminal amidation
  SITE 9: note = 3-(2-naphthyl)-L-alanine
  SITE 10: note = 2-methyl-L-leucine
DESCWQCWXXKNG

SEQ ID NO: 171 - protein, 12 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 12: note = C-terminal amidation
  SITE 9: note = 3-(2-naphthyl)-L-alanine
  SITE 10: note = 2-methyl-L-leucine
DESCWQCWXXKG

SEQ ID NO: 172 - protein, 14 aa, synthetic construct
  CROSSLNK 2..11: note = lactam bridge
  CROSSLNK 4..7: note = 1,3-dithio-propan-2-one bridge
  SITE 1: note = N-terminal acetylation
  SITE 14: note = C-terminal amidation
  SITE 9: note = 3-(2-naphthyl)-L-alanine
  SITE 10: note = 2-methyl-L-lysine
DESCWQCWXXKENG

SEQ ID NO: 173 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 1: note = N-terminal acetylation
  SITE 12: note = C-terminal amidation
  SITE 8: note = 3-(2-naphthyl)-L-alanine
  SITE 10: note = 2-methyl-L-leucine
DCQCWQCXWXCR

SEQ ID NO: 174 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 1: note = N-terminal acetylation
  SITE 12: note = C-terminal amidation
  SITE 10: note = 2-methyl-L-leucine
DCQCWQCYWXCR

SEQ ID NO: 175 - protein, 14 aa, synthetic construct
  CROSSLNK 2..11: note = 1,3-dithio-propan-2-one bridge
  CROSSLNK 4..7: note = 1,3-dithio-propan-2-one bridge
  SITE 1: note = N-terminal acetylation
  SITE 14: note = C-terminal amidation
  SITE 10: note = 2-methyl-L-leucine
DCQCWQCWWXCENG

SEQ ID NO: 176 - protein, 11 aa, synthetic construct
  CROSSLNK 1..10: note = lactam bridge
  CROSSLNK 3..6: note = lactam bridge
  SITE 1: note = (3-Aminomethyl)benzoyl
  SITE 11: note = C-terminal amidation
  SITE 3: note = L-diaminopropionic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSXWQEXXXER

SEQ ID NO: 177 - protein, 11 aa, synthetic construct
  CROSSLNK 1..10: note = lactam bridge
  CROSSLNK 3..6: note = lactam bridge
  SITE 1: note = (3-Aminomethyl)benzoyl
  SITE 11: note = C-terminal amidation
  SITE 6: note = L-diaminopropionic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSEWQXXXXER

SEQ ID NO: 178 - protein, 11 aa, synthetic construct
  CROSSLNK 1..10: note = lactam bridge
  CROSSLNK 3..6: note = lactam bridge
  SITE 1: note = (3-Aminomethyl)benzoyl
  SITE 11: note = C-terminal amidation
  SITE 6: note = diaminobutyric acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSEWQXXXXER

SEQ ID NO: 179 - protein, 11 aa, synthetic construct
  CROSSLNK 1..10: note = lactam bridge
  CROSSLNK 3..6: note = lactam bridge
  SITE 1: note = (3-Aminomethyl)benzoyl
  SITE 11: note = C-terminal amidation
  SITE 3: note = Orn also known as: L-ornithine or (2S)-2,5-diaminopentanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSXWQEXXXER

SEQ ID NO: 180 - protein, 11 aa, synthetic construct
  CROSSLNK 1..10: note = lactam bridge
  CROSSLNK 3..6: note = lactam bridge
  SITE 1: note = (3-Aminomethyl)benzoyl
  SITE 11: note = C-terminal amidation
  SITE 6: note = Orn also known as: L-ornithine or (2S)-2,5-diaminopentanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSEWQXXXXER

SEQ ID NO: 181 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 4: note = 7-Me-Trp also known as: (2S)-2-amino-3-(7-methyl-1H-indol-3-yl)propanoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCXQCXXXER

SEQ ID NO: 182 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 4: note = 7-Ph-Trp
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCXQCXXXER

SEQ ID NO: 183 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 4: note = 7-(Naphth-2-yl)-Trp
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
XSCXQCXXXER

SEQ ID NO: 184 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(3-Quinolinyl)-Ala
  SITE 9: note = 2-methyl-L-leucine
XSCWQCXXXER

SEQ ID NO: 185 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 6-Cl-Trp
  SITE 9: note = 2-methyl-L-leucine
XSCWQCXXXER

SEQ ID NO: 186 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 4-aminotetrahydro-2H-pyran-4-acetyl
XSCWQCXXXER

SEQ ID NO: 187 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = 3-(3-pyridyl)-L-alanine
XSCWQCXXXEX

SEQ ID NO: 188 - protein, 11 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoy
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = 3-(4-pyridyl)-L-alanine
XSCWQCXXXEX

SEQ ID NO: 189 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 12: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(3-pyridyl)-L-alanine
XSCWQCXXXERX

SEQ ID NO: 190 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 12: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = 3-(4-pyridyl)-L-alanine
XSCWQCXXXERX

SEQ ID NO: 191 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 11: note = C-terminal amidation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-arginine
KSCWQCXXXER

SEQ ID NO: 192 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
KTCWQCXXXER

SEQ ID NO: 193 - protein, 11 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
KSCWQCXXXES

SEQ ID NO: 194 - protein, 11 aa, synthetic construct
  CROSSLNK 1..10: note = lactam bridge
  CROSSLNK 3..6: note = 1,3-dithio-propan-2-one bridge
  SITE 1: note = 6-Aminohexanoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 195 - protein, 11 aa, synthetic construct
  CROSSLNK 1..10: note = lactam bridge
  CROSSLNK 3..6: note = 1,3-dithio-propan-2-one bridge
  SITE 1: note = 6-Amino-4-oxahexanoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 196 - protein, 11 aa, synthetic construct
  CROSSLNK 1..10: note = lactam bridge
  CROSSLNK 3..6: note = 1,3-dithio-propan-2-one bridge
  SITE 1: note = trans-4-Aminomethyl-cyclohexyl-1-carbonyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 2: note = beta-homo-serine
  SITE 11: note = D-arginine
XXCWQCXXXER

SEQ ID NO: 197 - protein, 11 aa, synthetic construct
  CROSSLNK 1..10: note = lactam bridge
  CROSSLNK 3..6: note = lactam bridge
  SITE 1: note = (4-(2-Aminoethyl)-piperazine-1-yl)-acetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 198 - protein, 11 aa, synthetic construct
  CROSSLNK 1..10: note = lactam bridge
  CROSSLNK 3..6: note = 1,3-dithio-propan-2-one bridge
  SITE 1: note = (4-(2-Aminoethyl)-piperazine-1-yl)-acetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXDR

SEQ ID NO: 199 - protein, 11 aa, synthetic construct
  CROSSLNK 1..10: note = lactam bridge
  CROSSLNK 3..6: note = 1,3-dithio-propan-2-one bridge
  SITE 1: note = 3-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 200 - protein, 11 aa, synthetic construct
  CROSSLNK 1..10: note = lactam bridge
  CROSSLNK 3..6: note = 1,3-dithio-propan-2-one bridge
  SITE 1: note = 2-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 201 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = N-terminal acetylation; note = K(N3)
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = L-propargylglycine
  SITE 11: note = D-arginine
XSCWQCXXXXR

SEQ ID NO: 202 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = Azidoacetic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = Glu(propargylamine)
  SITE 11: note = D-arginine
XSCWQCXXXXR

SEQ ID NO: 203 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = Azidoacetic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid(3-butyonic acid)
  SITE 11: note = D-arginine
XSCWQCXXXXR

SEQ ID NO: 204 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = but-3-ynoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid(azidoacetic acid)
  SITE 11: note = D-arginine
XSCWQCXXXXR

SEQ ID NO: 205 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = (N3)-Ala
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = Glu(propargylamine)
  SITE 11: note = D-arginine
XSCWQCXXXXR

SEQ ID NO: 206 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = (N3)-Ala
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid(3-butynoic acid)
  SITE 11: note = D-arginine
XSCWQCXXXXR

SEQ ID NO: 207 - protein, 11 aa, synthetic construct
  CROSSLNK 1..10: note = 1,4-disubstituted 1,2,3-triazole bridge
  CROSSLNK 3..6: note = 1,3-dithio-propan-2-one bridge
  SITE 1: note = but-3-ynoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid((N3)-alanine)
  SITE 11: note = D-arginine
XSCWQCXXXXR

SEQ ID NO: 208 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-2-pyridineacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 209 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-3-pyridineacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 210 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-2-fluoro-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 211 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-3-methyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 212 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-3-methoxy-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 213 - protein, 11 aa, synthetic construct
  CROSSLNK 1..10: note = 1,5-disubstituted 1,2,3-triazole bridge
  CROSSLNK 3..6: note = 1,3-dithio-propan-2-one bridge
  SITE 1: note = but-3-ynoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid(azidoacetic acid)
  SITE 11: note = D-arginine
XSCWQCXXXXR

SEQ ID NO: 214 - protein, 11 aa, synthetic construct
  CROSSLNK 1..10: note = 1,5-disubstituted 1,2,3-triazole bridge
  CROSSLNK 3..6: note = 1,3-dithio-propan-2-one bridge
  SITE 1: note = but-3-ynoic acid
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 10: note = (2S)-2,4-diaminobutanoic acid((N3)-alanine)
  SITE 11: note = D-arginine
XSCWQCXXXER

SEQ ID NO: 215 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-arginine
XSCWQCXXXER

SEQ ID NO: 216 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-2,4-Diaminobutanoyl([2-(trimethyl-2-aminoethoxy)e thoxy]propyl)
XSCWQCXXXEX

SEQ ID NO: 217 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 7: note = (2S)-2,4-diaminobutanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 218 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 7: note = 2,4-Diaminobutanoyl([2-(trimethyl-2-aminoethoxy)eth oxy]propyl)
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 219 - protein, 11 aa, synthetic construct
  CROSSLNK 1..10: note = lactam bridge
  CROSSLNK 3..6: note = 1,3-dithio-propan-2-one bridge
  SITE 1: note = 2,4-Diaminobutanoyl([2-(trimethyl-2-aminoethoxy)eth oxy]propyl)
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = D-arginine
XSCWQCXXXER

SEQ ID NO: 220 - protein, 11 aa, synthetic construct
  CROSSLNK 1..10: note = lactam bridge
  CROSSLNK 3..6: note = 1,3-dithio-propan-2-one bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = D-histidine
XSCWQCXXXEH

SEQ ID NO: 221 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 6: note = (2S)-2,4-diaminobutanoic acid
  SITE 8: note = 3-(2-naphthyl)-L-alanine
  SITE 9: note = 2-methyl-L-leucine
  SITE 11: note = C-terminal amidation
  SITE 11: note = (2S)-2,4-diaminobutanoic acid
XSEWQXVXXEX

SEQ ID NO: 222 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 6: note = (2S)-2,4-diaminobutanoic acid
  SITE 9: note = 2-methyl-L-leucine
  SITE 11: note = C-terminal amidation
  SITE 11: note = (2S)-2,4-diaminobutanoic acid
XSEWQXXHXEX

SEQ ID NO: 223 - protein, 11 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 6: note = (2S)-2,4-diaminobutanoic acid
  SITE 11: note = NH-3-aminopropanoyl
XSEWQXXXXEX

SEQ ID NO: 224 - protein, 11 aa, synthetic construct
  CROSSLNK 1..10: note = lactam bridge
  CROSSLNK 3..6: note = 1,3-dithio-propan-2-one bridge
  SITE 1: note = 4-Methylaminomethyl-phenylacetyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 11: note = C-terminal amidation
  SITE 11: note = (2S)-2,4-diaminobutanoic acid
XSCWQCXXXEX

SEQ ID NO: 225 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 11: note = (2S)-2,4-diaminobutanoic acid
  SITE 12: note = 3-(3-pyridyl)-L-alanine
XSCWQCXXXEXX

SEQ ID NO: 226 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = 3-(3-Quinolinyl)-alanine
XSCWQCXXXERX

SEQ ID NO: 227 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = D-3-(3-pyridyl)-L-alanine
XSCWQCXXXERX

SEQ ID NO: 228 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = 3-Amino-3-(3'-pyridyl)propionyl
XSCWQCXXXERX

SEQ ID NO: 229 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = F(3-F)
XSCWQCXXXERX

SEQ ID NO: 230 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = F(3,5-F)
XSCWQCXXXERX

SEQ ID NO: 231 - protein, 12 aa, synthetic construct
  CROSSLNK 1..10: note = lactam bridge
  CROSSLNK 3..6: note = 1,3-dithio-propan-2-one bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = 4-Aminomethyl-2-pyridineacetyl
XSCWQCXXXERX

SEQ ID NO: 232 - protein, 12 aa, synthetic construct
  CROSSLNK 1..10: note = lactam bridge
  CROSSLNK 3..6: note = 1,3-dithio-propan-2-one bridge
  SITE 1: note = N-terminal acetylation
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-leucine
  SITE 12: note = C-terminal amidation
  SITE 12: note = 3-(3-pyridyl)-L-alanine
  SITE 11: note = (2S)-2,4-diaminobutanoic acid
KTCWQCXXXEXX

SEQ ID NO: 233 - protein, 12 aa, synthetic construct
  CROSSLNK 1..10: note = lactam bridge
  CROSSLNK 3..6: note = lactam bridge
  SITE 1: note = (3-Aminomethyl)benzoyl
  SITE 7: note = (2S)-2-amino-3-[4-(2-aminoethoxy)phenyl]propanoic acid
  SITE 8: note = 3-(2-naphthyl)-L-alanine
  SITE 9: note = 2-methyl-L-valine
  SITE 12: note = C-terminal amidation
  SITE 6: note = (2S)-2,4-diaminobutanoic acid
  SITE 11: note = (2S)-2,4-diaminobutanoic acid
  SITE 12: note = 3-(3-pyridyl)-L-alanine
XIEWQXXXXEXX

SEQ ID NO: 234 - protein, 12 aa, synthetic construct
  CROSSLNK 1..10: note = lactam bridge
  CROSSLNK 3..6: note = lactam bridge
  SITE 1: note = 4-Aminomethyl-phenylacetyl
  SITE 7: note = L-2-phenylglycine
  SITE 8: note = F(3,4-Me)
  SITE 9: note = 2-methyl-L-valine
  SITE 12: note = C-terminal amidation
  SITE 6: note = (2S)-2,4-diaminobutanoic acid
  SITE 11: note = (2S)-2,4-diaminobutanoic acid
  SITE 12: note = 2-Me-3-(3-Pyridyl)-alanine
XIEWQXXXXEXX

SEQ ID NO: 235 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 12: note = C-terminal amidation
GCTCWECWWLCS

SEQ ID NO: 236 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 12: note = C-terminal amidation
DCSCWQCWWLCR

SEQ ID NO: 237 - protein, 13 aa, synthetic construct
  CROSSLNK 2..12: note = 1,3-dithio-propan-2-one bridge
  CROSSLNK 5..10: note = 1,3-dithio-propan-2-one bridge
  SITE 12: note = C-terminal amidation
ECWLCYYRNCNCM

SEQ ID NO: 238 - protein, 12 aa, synthetic construct
  CROSSLNK 2..11: note = 1,3-dithio-propan-2-one bridge
  CROSSLNK 4..7: note = 1,3-dithio-propan-2-one bridge
  SITE 10: note = 2-methyl-L-leucine
  SITE 12: note = C-terminal amidation
GCTCWECWWXCS

SEQ ID NO: 239 - protein, 12 aa, synthetic construct
  CROSSLNK 1..6: note = thioether bridge
  SITE 1: note = (2S)-2-aminobutyric acid
  SITE 7: note = (2S)-2-amino-3-[4-(2-aminoethoxy)phenyl]propanoic acid
  SITE 9: note = 2-methyl-L-lysine
  SITE 12: note = C-terminal amidation
  SITE 1: note = N-terminal acetylation
XQTWQCXWXENG

SEQ ID NO: 240 - protein, 14 aa, synthetic construct
  CROSSLNK 2..7: note = thioether bridge
  SITE 2: note = (2S)-2-aminobutyric acid
  SITE 7: note = (2S)-2-amino-3-[4-(2-aminoethoxy)phenyl]propanoic acid
  SITE 8: note = 3-(2-naphthyl)-L-alanine
  SITE 9: note = 4-Aminotetrahydro-2H-pyran-4-carbonyl
  SITE 14: note = C-terminal amidation
  SITE 1: note = N-terminal acetylation
  SITE 1: note = D-arginine
XRXQTWQCXXXENN